(19) United States
(12) Patent Application Publication
(10) Pub. No.: US 2024/0158864 A1
(43) Pub. Date: May 16, 2024

(54) METHODS AND COMPOSITIONS FOR IDENTIFYING NEUROENDOCRINE PROSTATE CANCER

(71) Applicant: Dana-Farber Cancer Institute, Inc., Boston, MA (US)

(72) Inventors: Jacob Berchuck, Brookline, MA (US); Matthew Freedman, Needham, MA (US); Sylvan Baca, Brookline, MA (US)

(21) Appl. No.: 18/272,184

(22) PCT Filed: Jan. 24, 2022
(86) PCT No.: PCT/US22/13462

§ 371 Date: Jul. 13, 2023

Related U.S. Application Data
(60) Provisional application No. 63/288,283, filed on Dec. 10, 2021.
(60) Provisional application No. 63/141,108, filed on Jan. 25, 2021.

(51) Int. Cl. C12Q 1/6886; C12Q 1/6874

(52) U.S. Cl. CPC C12Q 1/6886; C12Q 1/6874; C12Q 1/6806; C12Q 2600/154

(57) ABSTRACT

Methods and compositions are provided for detecting the presence of neuroendocrine prostate cancer in a subject by analyzing DNA methylomes.

### CROSS-REFERENCE TO RELATED APPLICATIONS

This application is the U.S. national phase of International Patent Application No. PCT/US2022/013462, filed on 24 Jan. 2022, which claims the benefit of priority to U.S. Provisional Application Ser. No. 63/141,108, filed on 25 Jan. 2021; and U.S. Provisional Application Ser. No. 63/288,283, filed on 10 Dec. 2021; the entire contents of each of said applications are incorporated herein in their entirety by this reference.

### STATEMENT OF RIGHTS

This invention was made with government support under grant number W81XWH-20-1-0118 awarded by the Department of Defense. The government has certain rights in the invention.

### BACKGROUND OF THE INVENTION

Prostate adenocarcinoma (PRAD) cells can trans-differentiate to NEPC as a resistance mechanism to potent androgen receptor signaling inhibitors (ARSIs) (Ku, S. Y. et al. (2017) Science 355 78-83; Mu, P. et al. (2017) Science 355: 84-88). NEPC emerges in 11-17% of men with metastatic prostate cancer and is associated with poor responsiveness to ARSIs and shorter survival (Abida, W. et al. (2019) Proc. Natl. Acad. Sci. 116: 11428-11436. Aggarwal, R. et al. (2018) J. Clin. Oncol. 36: 2492-2503). In contrast, men with NEPC are more likely to respond to platinum-based chemotherapy, highlighting the clinical and therapeutic importance of detecting this phenotype (Humeniuk, M. S. et al. (2018) Prostate Cancer Prostatic Dis. 21: 92-99).

The current approach to diagnosing NEPC has significant shortcomings. The standard of care is to perform tissue biopsy for pathologic tumor analysis. However, optimal timing of a biopsy is not established and there is a lack of consensus pathological criteria for defining NEPC. Further, it is well established that due to intra-patient tumor heterogeneity, biopsy samples may not be representative of metastatic prostate cancer patients' overall disease (Beltran, H. et al. (2016) Nat. Med. 22: 298-305; Gundem, G. et al. (2015) Nature 520: 353-357; Beltran, H. et al. (2020) J. Clin. Invest. 130: 1653-1668). Because NEPC emerges as a treatment-resistant subclone, depending on when a biopsy is performed, the bulk of a patient's tumor burden may be PRAD. Consequently, NEPC diagnosis is often delayed or missed and reported rates likely underestimate the true prevalence of this aggressive disease variant.

Liquid biopsies have the potential to address this unmet need. Clinical cfDNA tests generally focus on detection of somatically acquired tumor mutations and/or copy number alterations. However, as NEPC arises clonally from PRAD, genetic alterations in NEPC are not specific to this resistance phenotype. Consequently, the defining genetic hallmark of NEPC, deleterious alterations in RB1 and/or TP53, are present in more than one-third of castration-resistant PRAD tumors and thus cannot be used to detect NEPC. DNA methylation profiles of NEPC and PRAD tumors demonstrate striking differences (Beltran, H. et al. (2016) Nat. Med. 22: 298-305). These NEPC-associated DNA methylation changes have been detected in plasma cfDNA using whole-genome bisulfite sequencing with high concordance with matched tumor biopsies (Beltran, H. et al. (2020) J. Clin. Invest. 130, 1653-1668). As a result of widespread epigenetic reprogramming that occurs during trans-differentiation from PRAD to NEPC, these global DNA methylation differences present a promising diagnostic opportunity. Additional biomarkers are needed to provide more reliable diagnoses of prostate cancer subtypes. Such biomarkers will allow treatment options tailored to the particular prostate cancer subtype.

### SUMMARY OF THE INVENTION

The present invention is based, at least in part, on the discovery of particular differentially methylated regions (DMRs) of the genome in subjects with neuroendocrine prostate cancer (NEPC) relative to subjects with prostate adenocarcinoma (PRAD). The present disclosure represents the first application of cfMeDIP-seq to detect a clinically actionable resistance phenotype early in the disease history based on the distinct methylomes of PRAD and NEPC with high accuracy and sensitivity. The identified DMRs can be used to diagnose subjects with NEPC or PRAD and determine treatment options.

One aspect of the present invention provides a method for determining if a subject has or is at risk for developing neuroendocrine prostate cancer (NEPC), the method comprising detecting the presence or absence of altered methylation relative to a control of one or more of the genomic loci listed in Table 5 in the genomic DNA (gDNA), cell free DNA (cfDNA), and/or circulating tumor DNA (ctDNA) in a sample derived from the subject, wherein the presence of altered methylation of the one or more of the genomic loci indicates that the subject has or is at risk for developing NEPC.

Another aspect provides a method for treating a subject having or suspected of having NEPC, the method comprising administering to the subject a therapeutically effective amount of an agent that modulates the methylation of one or more of the genomic loci listed in Table 5.

Numerous embodiments are further provided that can be applied to any aspect of the present invention and/or combined with any other embodiment described herein. For example, in one embodiment, the method further comprises obtaining a biological sample from the subject. In another embodiment, detecting the presence or absence of methylation comprises determining the level of methylation of the one or more genomic loci. In still another embodiment, the method further comprises generating a methylation profile from the detected presence, absence, or level of methylation at the one or more genomic loci listed in Table 5. In yet another embodiment, the method further comprises comparing the presence, absence, and/or level of methylation at the one or more of the genomic loci listed in Table 5 or the methylation profile to a control. In one embodiment, the methylation or absence of methylation at the one or more genomic loci listed in Table 5 is detected by cell-free methylated DNA immunoprecipitation and high-throughput sequencing (cfMeDIP-seq). In another embodiment, the presence or absence of methylation at the one or more DMRs listed in Table 5 is detected by whole genome bisulfite sequencing (WGBS). In yet another embodiment, at least one of the genomic loci comprises between about 50 and about 1000 nucleotides. In still another embodiment, at least one of the genomic loci comprises between about 50 and about 500 nucleotides. In one embodiment, at least one of the genomic loci comprises about 300 nucleotides. In another embodiment, the one or more genomic loci listed in Table 5 comprises between 1,112 and 1,674, between 124 and 193 genomic loci, between 51 and 76 genomic loci, or between 17 and 20 genomic loci. In yet another embodiment, the 1,112 genomic loci are listed in Table 1, the 124 genomic loci are listed in Table 2, the 51 genomic loci are listed in Table 3, the 17 genomic loci are listed in Table 4, the 193 genomic loci are listed in Table 6, the 76 genomic loci are listed in Table 7, and the 20 genomic loci are listed in Table 8. In still another embodiment, the genomic loci are differentially methylated regions (DMRs) relative to the same regions in a tissue control sample or a sample derived from a subject having or at risk of developing prostate adenocarcinoma (PRAD). In one embodiment, the genomic loci have a predetermined area under the ROC curve (AUROC) of greater than 0.7. In another embodiment, the one or more the genomic loci have increased methylation relative to the same region in a tissue control sample or a sample derived from a subject having or at risk of developing PRAD. In still another embodiment, the one or more the genomic loci have less methylation relative to the same region in a tissue-control sample or a sample derived from a subject having or at risk of developing PRAD. In yet another embodiment, the method further comprises determining a methylation score for the one or more genomic loci and/or the methylation profile. In one embodiment, the method further comprises comparing the methylation score for the one or more genomic loci and/or the methylation profile to a predetermined threshold for each of the one or more genomic loci listed in Tables 1-8 or to a predetermined threshold for the methylation profile. In another embodiment, the predetermined threshold discriminates between NEPC and PRAD. In still another embodiment, the method further comprises comparing the methylation score to a control, wherein a higher methylation score compared to the control indicates that the subject has or is at risk for developing NEPC. In yet another embodiment, the control is a reference value. In one embodiment, the control is a methylation score determined from a control sample. In another embodiment, the control sample is obtained from a subject without NEPC. In yet another embodiment, the control sample is obtained from a subject with NEPC. In still another embodiment, the sample is selected from the group consisting of organs, tissue, body fluids, and cells. In one embodiment, the body fluid is selected from the group consisting of whole blood, serum, plasma, sputum, spinal fluid, lymph fluid, skin secretions, respiratory secretions, intestinal secretions, genitourinary tract secretions, tears, buccal scrape, saliva, cerebrospinal fluid, urine, and stool. In another embodiment, the bodily fluid is whole blood, serum, or plasma. In yet another embodiment, the method further comprises isolating cell-free DNA (cfDNA) or circulating tumor DNA (ctDNA) isolated from plasma obtained from the subject.

Neuroendocrine prostate cancer (NEPC) is a resistance phenotype that emerges in men with metastatic castration-resistant prostate adenocarcinoma (CR-PRAD). Early detection of neuroendocrine prostate (NEPC) is challenging in clinical practice, but has important prognostic and therapeutic implications for patients with metastatic castration-resistant prostate cancer (mCRPC).

The invention provided herein, is also related, in part, to methods of generating an NEPC risk score. In the largest study to date of cell-free DNA (cfDNA) from men with NEPC, the data shown herein provide a validated non-invasive NEPC Risk Score through tissue-informed cell-free DNA methylation analysis. Applying the NEPC Risk Score to cfDNA from two independent cohorts of men with mCRPC resulted in highly accurate discrimination between men with versus men without NEPC. In both cfDNA cohorts, high NEPC Risk Score was associated with significantly worse overall survival. The data included herein show the clinical utility of the cfDNA methylation-based NEPC Risk Score in men with mCRPC to non-invasively identify those who should be considered for platinum-based chemotherapy or clinical trials of novel NEPC-directed therapies.

From cell-free methylated DNA immunoprecipitation sequencing (cfMeDIP-seq) data, a NEPC Methylation Value and PRAD Methylation Value for each sample is calculated by summing the methylated cfDNA fragments at tissue-derived NEPC-enriched and PRAD-enriched DMRs, respectively (FIG. 3A). An NEPC Risk Score may be calculated for each sample as the normalized ratio of the NEPC Methylation Value versus the PRAD Methylation Value.

Therefore, provided herein are methods of determining if a subject with prostate cancer has or is at risk for developing neuroendocrine prostate cancer (NEPC), the method comprising generating an NEPC Risk Value score for the subject, wherein an NEPC Risk Score of greater than or equal to 0, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, or 0.5 indicates that the subject has or is at risk for developing NEPC.

In some aspects, provided herein are methods of determining if a subject with prostate cancer would benefit from platinum-based chemotherapy, the method comprising generating an NEPC Risk Value score for the subject, wherein an NEPC Risk Score of greater than or equal to 0, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, or 0.5 indicates that the subject would benefit from platinum-based chemotherapy.

In some embodiments, the NEPC Risk Value is the log₂ ratio of a NEPC Methylation Value to a PRAD Methylation Value.

In certain embodiments, the NEPC Methylation Value is calculated by summing relative methylation scores of at least two NEPC-enriched differentially methylated regions in DNA from a sample taken from the subject.

The NEPC Methylation Value may be calculated by summing relative methylation scores of at least 3, at least 9, at least 17, at least 20, at least 76 at least 124, at least 193, at least 479, at least 504, at least 1112, at least 1674, at least 3498, at least 3523, at least 5552, or at least 5604 NEPC-enriched differentially methylated regions in DNA from a sample taken from the subject. The NEPC Methylation Value may be calculated by summing relative methylation scores of at least 17, at least 51, at least 124, or at least 1112 NEPC-enriched differentially methylated regions in DNA from a sample taken from the subject. In some embodiments, the relative methylation scores (rms) are calculated by taking the sum of relative methylation scores at each site, and dividing by the sum of relative methylation scores across all sites in the genome.

In some embodiments, the relative methylation scores are calculated by the R package MEDIPS as described on the World Wide Web at genome.cshlp.org/content/suppl/2010/08/03/gr.110114.110.DC1/Chavez_GR-110114_Supplementary_Methods.pdf and Example 10. In some embodiments, the NEPC Methylation Value is normalized to CpG content of the local sequence.

In some embodiments, the NEPC-enriched differentially methylated regions have a predetermined area under the ROC curve (AUROC) of greater than 0.8, greater than 0.9, greater than 0.95, or greater than 0.99.

The NEPC-enriched differentially methylated regions may comprise any one of the genomic loci listed in any one of Tables 1-8 or 12-15.

In certain embodiments, the PRAD Methylation Value is calculated by summing relative methylation scores of at least two PRAD-enriched differentially methylated regions in DNA from a sample taken from the subject.

The PRAD Methylation Value may be calculated by summing relative methylation scores of at least 14, at least 33, at least 42, at least 100, at least 277, at least 783, at least 1600, at least 2347, at least 5405, at least 7287, at least 7288, at least 15590, at least 18943, at least 21688, or at least 26209 PRAD-enriched differentially methylated regions in DNA from a sample taken from the subject. The PRAD Methylation Value may be calculated by summing relative methylation scores of at least 76, at least 212, at least 590, or at least 5404 PRAD-enriched differentially methylated regions in DNA from a sample taken from the subject. In some embodiments, the relative methylation scores (rms) are calculated by taking the sum of relative methylation scores at each site, and dividing by the sum of relative methylation scores across all sites in the genome.

In some embodiments, the relative methylation scores are calculated by the R package MEDIPS as described on the World Wide Web at genome.cshlp.org/content/suppl/2010/08/03/gr.110114.110.DC1/Chavez_GR-110114_Supplementary_Methods.pdf and Example 10. In some embodiments, the PRAD Methylation Value is normalized to CpG content of the local sequence.

In some embodiments, the PRAD-enriched differentially methylated regions have a predetermined area under the ROC curve (AUROC) of greater than 0.8, greater than 0.9, greater than 0.95, or greater than 0.99.

The PRAD-enriched differentially methylated regions may comprise any one of the genomic loci listed in any one of Tables 16-27.

In some embodiments, a kit for determining if a subject with prostate cancer has or is at risk for developing neuroendocrine prostate cancer (NEPC), the kit comprising a reagent for detecting the presence, absence, or level of methylation in the genomic DNA or cell free DNA (cfDNA) in a sample, or circulating tumor DNA (ctDNA) wherein the methylation profile comprises one or more of the genomic loci listed in any one of Tables 1-8 and 12-27.

Also provided herein are methods of determining the progression of prostate cancer in a patient in need thereof by calculating a NEPC Risk Score as described herein.

In another embodiment, the control sample is obtained from a subject without NEPC. In yet another embodiment, the control sample is obtained from a subject with NEPC.

In still another embodiment, any sample provided herein is a sample is selected from the group consisting of organs, tissue, body fluids, and cells. In one embodiment, the body fluid is selected from the group consisting of whole blood, serum, plasma, sputum, spinal fluid, lymph fluid, skin secretions, respiratory secretions, intestinal secretions, genitourinary tract secretions, tears, buccal scrape, saliva, cerebrospinal fluid, urine, and stool. In another embodiment, the bodily fluid is whole blood, serum, or plasma. In yet another embodiment, the method further comprises isolating cell-free DNA (cfDNA) or circulating tumor DNA (ctDNA) isolated from plasma obtained from the subject.

In still another embodiment, the method further comprises administering to the subject a therapeutically effective amount of an anti-cancer therapy. In one embodiment, the anti-cancer therapy one or more of the therapies selected from the group consisting of an epigenetic modifier, targeted therapy, chemotherapy, radiation therapy, immunotherapy, and/or hormonal therapy. In another embodiment, the subject is resistant to AR-targeted therapy. In yet another embodiment, the chemotherapy is a platinum-based therapy. In still another embodiment, the chemotherapy further comprises etoposide. In one embodiment, the chemotherapy is doxorubicin, etoposide, or cisplatin or combination thereof. In another embodiment, the anti-cancer therapy is an immunotherapy. In yet another embodiment, the immunotherapy is cell-based. In still another embodiment, the immunotherapy comprises a cancer vaccine and/or virus. In one embodiment, the immunotherapy comprises an immune checkpoint inhibitor. In another embodiment, the immune checkpoint inhibitor inhibits a checkpoint selected from the group consisting of CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRPalpha (CD47), CD48, 2B4 (CD244), B7.1, B7.2, ILT-2, ILT-4, TIGIT, HHLA2, butyrophilins, and A2aR. In yet another embodiment, the immune checkpoint is PD1, PD-L1, or CD47. In still another embodiment, the immune checkpoint inhibitor is one or more monoclonal antibody. In one embodiment, the one or more monoclonal antibody is durvalumab. In another embodiment, the one or more monoclonal antibody is atezolizumab. In yet another embodiment, the one or more monoclonal antibody is atezolizumab and durvalumab. In still another embodiment, the one or more monoclonal antibody is pembrolizumab. In one embodiment, the immunotherapy is administered in combination with a chemotherapy. In another embodiment, the chemotherapy is a platinum-based chemotherapy. In yet another embodiment, the anti-cancer therapy is administered in a pharmaceutically acceptable formulation.

Another aspect of the present invention is a method for monitoring the progression of prostate cancer in a subject, the method comprising a) determining in a subject sample at a first point in time the level of altered methylation relative to a control of one or more of the genomic loci listed in Table 5 in the genomic DNA, cell free DNA (cfDNA), or circulating tumor DNA (ctDNA) in a sample derived from the subject; b) determining in a subject sample at least one subsequent point in time the level of altered methylation relative to a control of one or more of the genomic loci listed in Tables 1-8 in the genomic DNA, cell free DNA (cfDNA), or circulating tumor DNA (ctDNA) in a sample derived from the subject; and c) comparing the aggregate level of methylation determined in steps a and b, thereby monitoring the progression of NEPC in the subject.

Yet another aspect provides a method of assessing the efficacy of an agent for treating NEPC in a subject, the method comprising determining in a subject sample at a first point in time the level of altered methylation relative to a control of one or more of the genomic loci listed in Table 5 in the genomic DNA, cell free DNA (cfDNA), or circulating tumor DNA (ctDNA) in a sample derived from the subject; determining in a subject sample at least one subsequent point in time the level of altered methylation relative to a control of one or more of the genomic loci listed in Tables 1-8 in the genomic DNA, cell free DNA (cfDNA), or circulating tumor DNA (ctDNA) in a sample derived from the subject; wherein an increased aggregate level of methylation determined in the subsequent sample relative to the aggregate level of methylation detected the first sample indicates that the agent does not treat NEPC in the subject; and wherein a decreased aggregate level of methylation determined in the subsequent sample relative to the aggregate level of methylation detected the first sample indicates that the agent treats NEPC in the subject.

In one embodiment, the one or more genomic loci listed in Table 5 comprises between 1,112 and 1,674, between 124 and 193 genomic loci, between 51 and 76 genomic loci, or between 17 and 20 genomic loci. In another embodiment, the 1,112 genomic loci are listed in Table 1, the 124 genomic loci are listed in Table 2, the 51 genomic loci are listed in Table 3, the 17 genomic loci are listed in Table 4, the 193 genomic loci are listed in Table 6, the 76 genomic loci are listed in Table 7, and the 20 genomic loci are listed in Table 8. In yet another embodiment, between the first point in time and the subsequent point in time, the subject has undergone treatment, completed treatment, and/or is in remission for NEPC. In still another embodiment, the first and/or at least one subsequent sample is an ex vivo or an in vivo sample. In one embodiment, the first and/or at least one subsequent sample is obtained from an animal model of NEPC. In another embodiment, the first and/or at least one subsequent sample is a portion of a single sample or pooled samples obtained from the subject. In yet another embodiment, the sample comprises cells, cell lines, histological slides, paraffin embedded tissue, fresh frozen tissue, fresh tissue, biopsies, blood, plasma, serum, buccal scrape, saliva, cerebrospinal fluid, urine, stool, mucus, bone marrow, peritumoral tissue, and/or intratumoral tissue obtained from the subject. In still another embodiment, the sample is whole blood, serum, or plasma. In one embodiment, the method further comprises isolating the gDNA, cfDNA, and/or ctDNA.

Another aspect provides a method for identifying an agent that inhibits NEPC cancer cell activity comprising contacting the NEPC cancer cell with a test agent and detecting reduced methylation of one or more of the genomic loci listed in Table 5. In one embodiment, the one or more genomic loci listed in Table 5 comprises between 1,112 and 1,674, between 124 and 193 genomic loci, between 51 and 76 genomic loci, or between 17 and 20 genomic loci. In another embodiment, the 1,112 genomic loci are listed in Table 1, the 124 genomic loci are listed in Table 2, the 51 genomic loci are listed in Table 3, the 17 genomic loci are listed in Table 4, the 193 genomic loci are listed in Table 6, the 76 genomic loci are listed in Table 7, and the 20 genomic loci are listed in Table 8. In yet another embodiment, contacting the NEPC cancer cell occurs in vivo, ex vivo, or in vitro.

In one embodiment of the foregoing methods, the subject is an animal model of NEPC. In another embodiment, the animal model is a rodent model. In yet another embodiment, the subject is a mammal. In still another embodiment, the mammal is a mouse or a human.

Yet another aspect provides a kit for assessing the ability of an agent to treat NEPC, the kit comprising a reagent for detecting the presence, absence, or level of methylation in the genomic DNA or cell free DNA (cfDNA) in a sample, or circulating tumor DNA (ctDNA) wherein the methylation profile comprises one or more of the genomic loci listed in Tables 1-8 and/or Tables 12-27.

Another aspect provides a kit for determining if a subject has or is at risk for developing NEPC, the kit comprising a reagent for determining the presence, absence, or level of methylation of one or more the genomic loci listed in Tables 1-8 and/or Tables 12-27.

Also provided herein are methods of measuring or determining the level of altered methylation (e.g., relative to a control) of one or more of the genomic loci listed in Tables 12-27 in the genomic DNA. Any method described herein which determines the presence, absence, or level of methylation of one or more of the genomic loci listed in Tables 1-8 may also include determining the presence, absence, or level of methylation of one or more of the genomic loci listed in Tables 12-27.

### BRIEF DESCRIPTION OF THE DRAWINGS

The patent of application file contains at least one drawing executed in color. Copies of this patent or patent application publication with color drawings will be provided by the office upon request and payment of the necessary fee.

FIG. 1A—FIG. 1F demonstrate the classification of neuroendocrine prostate cancer (NEPC) versus prostate adenocarcinoma (PRAD) based on cfDNA methylation. FIG. 1A shows boxplots of histology classification scores for NEPC versus PRAD samples. Box plots are displayed with a median center line, box range from the 25th to 75th percentile and whiskers extending to the most extreme observation within 1.5 times the interquartile range. P-value corresponds to Wilcoxon rank-sum tests. FIG. 1B is a ROC curve demonstrating accurate classification of NEPC versus PRAD samples. FIG. 1C is a volcano plot of patient derived xenograft (PDX) NEPC-enriched (N=39,699) and PRAD-enriched (N=137,692) differentially methylated regions (DMRs). Darker dots represent DMRs with q-value<0.05. FIG. 1D is a graph illustrating the correlation between PDX DMRs with differentially methylated nucleotides in reduced representation bisulfite sequencing (RRBS) data from castration-resistant NEPC and PRAD tumors. FIG. 1E is boxplots of the NEPC enrichment score for NEPC versus PRAD samples using tissue-informed classification. FIG. 1F is an ROC curve demonstrating accurate classification of NEPC versus PRAD samples using tissue-informed classification.

FIG. 2A—FIG. 2H characterize different subsets of DMRs. FIG. 2A is an ROC curve for a set of 1,112 DMRs using a log₂ fold-difference threshold >2 and FDR-adjusted p-value<0.001. FIG. 2B comprises box plots showing normalized summed relative methylation scores in cfDNA at 1,112 NEPC-enriched PDX DMRs for each sample from patients with PRAD versus patients with NEPC (p=7×10⁻⁵ (Wilcoxon rank sum test)). FIG. 2C is an ROC curve for a set of 124 DMRs using a log₂ fold-difference threshold >3 and FDR-adjusted p-value<10⁻⁵. FIG. 2D comprises boxplots showing normalized summed relative methylation scores in cfDNA at 124 NEPC-enriched PDX DMRs for each sample from patients with PRAD versus patients with NEPC (p=1.9×10⁻⁴ (Wilcoxon rank sum test)). FIG. 2E is an ROC curve for a set of 51 DMRs using a log₂ fold-difference threshold >3 and FDR-adjusted p-value<10⁻⁶. FIG. 2F comprises boxplots showing normalized summed relative methylation scores in cfDNA at 51 NEPC-enriched PDX DMRs for each sample from patients with PRAD versus patients with NEPC (p=5.5×10⁻⁵ (Wilcoxon rank sum test)). FIG. 2G is an ROC curve for a set of 17 DMRs using a log₂ fold-difference threshold >3 and FDR-adjusted p-value<10⁻⁷. FIG. 2H comprises boxplots showing normalized summed relative methylation scores in cfDNA at 17 NEPC-enriched PDX DMRs for each sample from patients with PRAD versus patients with NEPC (p=0.015 (Wilcoxon rank sum test)).

FIG. 3A-FIG. 3E show identification of tumor-derived PRAD-enriched and NEPC-enriched DMRs. FIG. 3A shows an overview of the methods used to detect the presence of NEPC based on tissue-informed cfDNA analysis. FIG. 3B shows a volcano plot showing differentially methylated regions (DMRs) between PRAD (N=24) and NEPC (N=5) patient-derived xenografts. Red and blue dots represent NEPC-enriched PRAD-enriched (N=137,692) and NEPC-enriched (N=39,699) DMRs, respectively, with FUR-adjusted P<0.05. FIG. 3C shows a correlation between tumor-derived DMRs with differentially methylated nucleotides in reduced representation bisulfite sequencing (RRBS) data from CR-PRAD and NEPC tumors. FIG. 3D shows methylation at the SPDEF gene and UNC13A gene determined by MeDIP-seq in PRAD tumors, NEPC tumors, and white blood cells (WBCs). FIG. 3E shows the top 5 gene ontology (GO) enrichment terms for PRAD-enriched and NEPC-enriched DMRs after removing sites with DNA methylation in WBCs.

FIG. 4A-FIG. 4D show classification of NEPC and PRAD samples in the cfDNA test cohort. NEPC Methylation Values (FIG. 4A), PRAD Methylation Values (FIG. 4B), and NEPC Risk Scores (FIG. 4C) in cfDNA samples from men with PRAD or NEPC in the test cohort. P-Values were calculated using a two-sided Wilcoxon rank-sum test. Optimal cutoff (indicated by dotted gray line) was determined in this cohort using Youden's J statistic. FIG. 4D shows the Kaplan-Meier curve for overall survival (OS) from the time of metastatic disease for men with high (>0.15) versus low (<0.15) NEPC Risk Score relative to the cutoff.

FIG. 5A-FIG. 5D show classification of NEPC and PRAD samples in the cfDNA validation cohort. NEPC Methylation Values (FIG. 5A), PRAD Methylation Values (FIG. 5B), and NEPC Risk Scores (FIG. 5C) in cfDNA samples from men with NEPC or PRAD in the validation cohort are shown. P-Values were calculated using a two-sided Wilcoxon rank-sum test. The optimal NEPC Risk Score cutoff determined in the independent cfDNA test cohort is indicated by dotted gray line. FIG. 5D shows Kaplan-Meier curve for overall survival (OS) from the time of metastatic disease for men with high (>0.15) versus low (≤0.15) NEPC Risk Score relative to the cutoff determined in the independent cfDNA test cohort.

FIG. 6 shows cfDNA from men with CR-PRAD with high NEPC Risk Scores display clinical and genomic features of NEPC.

FIG. 7A-FIG. 7G show the association of the plasma cfDNA methylome with NEPC Risk Score and tumor content. FIG. 7A shows a principal component analysis (PCA) of the genome-wide methylome for 101 plasma cfDNA samples from men with CR-PRAD or NEPC. FIG. 7B shows a PCA of the 101 plasma cfDNA samples limiting to the NEPC- and PRAD-enriched DMRs included in the NEPC Risk Score. Correlation between NEPC Risk Score with the top 10 principal components (PCs) for the cfDNA genome-wide methylome data (FIG. 7C) and restricted to the DMR sites (FIG. 7D) is shown. Correlation between cfDNA tumor content with the top 10 PCs for the cfDNA genome-wide methylome data (FIG. 7E) and restricted to the DMR sites (FIG. 7F) is shown. Correlation between NEPC Risk Score and each PC was measured using the coefficient of determination (R ²). * P<0.05; ** P<1×10⁻⁶. FIG. 7G shows a correlation between NEPC Risk Score and tumor content for the 101 cfDNA samples from men with NEPC and CR-PRAD. Dotted lines show the linear regression for the NEPC samples (red), CR-PRAD samples (blue), and all samples (purple).

FIG. 8 shows various log₂-fold change cutoffs and FDR-adjusted p value cutoffs for defining differentially methylated regions (DMRs) in NEPC vs PRAD tissue. The number of up peaks (more methylated in NEPC) and down peaks (more methylated in PRAD) is shown for each cutoff. The area under the ROC curve for classifying plasma using each DMR set is indicated.

FIG. 9 shows a consort diagram for cfDNA samples. Low-pass whole-genome sequencing on cfDNA samples from 129 men, 102 with PRAD and 27 with NEPC, was performed. These patients comprised two independent cohorts including 56 in the test cohort (45 PRAD and 11 NEPC) and 73 in the validation cohort (57 PRAD and 16 NEPC). LPWGS was done first and ichorCNA was utilized to estimate cfDNA tumor content for each sample. Samples with undetectable cfDNA tumor content (less than 3%) using ichorCNA were excluded from subsequent cfDNA methylation analysis. Six PRAD and two NEPC samples were excluded from the test cohort and 16 PRAD and 4 NEPC samples were excluded from the validation cohort based on undetectable cfDNA tumor content.

FIG. 10A-FIG. 10B shows cfDNA tumor content in the test and validation cohorts. Estimated cfDNA tumor content in cfDNA samples from men with PRAD or NEPC in the test (FIG. 10A) and validation (FIG. 10B) cohorts. P-Values calculated using a two-sided Wilcoxon rank-sum test.

FIG. 11 shows overall survival based on cfDNA tumor content. Kaplan-Meier curve for overall survival from the time of metastatic disease for men by tertile of cfDNA tumor content. Hazard ratio of the 2^{nd} and 3^{rd} tertile relative to the 1^{st} tertile is reported.

FIG. 12A-FIG. 12B shows percent of variance in methylation data explained by the top ten principal components. Percent of variance explained by each principal component in the principal component analysis of the genome-wide methylome data (FIG. 12A) and the methylation data at the NEPC- and PRAD-enriched DMRs (FIG. 12B) for the 101 cfDNA samples from men with NEPC or CR-PRAD included in the NEPC Risk Score analysis.

FIG. 13 shows calculation of coupling factors for Example 10. The upper panel shows a schematic view of the genome vector created by defining a bin size of 50 bp. In addition, CpGs are shown in a schematic way. A coupling factor is calculated for the centered genomic bin at position b (marked by a red vertical line). For this, all CpGs within a maximal distance d are considered. The maximal distance d reflects the estimated average size of sequenced DNA fragments. There are several ways for calculating coupling factors. The simplest way is to count the number of CpGs in the surrounding of b but with a maximal distance of d. Alternatively, a weighting function can be applied to weight each CpG by its distance (dist) to the current genomic bin at position b. Again, there are several possible weighting functions. The five images at the bottom of the Figure show the progression of the weighting functions linear, exp, log, count, and custom (Down et al. 2008) by defining d=700.

FIG. 14A-FIG. 14B shows evaluation of coupling factor calculations for Example 10. FIG. 14A shows the resulting Pearson correlations (y-axis) between the mean coupling factors and bisulphite sequencing derived mean methylation values for a varying distance parameter d (x-axis) and for different weighting factors (colours). The best negative correlation (−0.73) was achieved by setting the parameter d=700 and by using the count function. FIG. 14B shows the according scatterplot where each data point represents a HEP trace. The scatterplot contrasts the mean methylation value (x-axis) and mean CpG denisty (y-axis). The color code divides the full range of CpG densities into quantiles.

FIG. 15 shows global mean rpm signal distributions for Example 10. The figure illustrates histograms for the mean rpm values of all genome-wide overlapping 500 bp windows for hESCs, DE, and input samples. The grey lines indicate three possible global rpm thresholds as derived by setting the qt parameter to qt=0.9, qt=0.95, and qt=0.99.

### DETAILED DESCRIPTION OF THE INVENTION

As demonstrated herein, detection and analysis of differentially methylated regions (DMRs) of the human genome can be used to diagnose neuroendocrine prostate cancer (NEPC) as well as discriminate between NEPC and prostate adenocarcinoma (PRAD). In particular, methylation profiles that include one or more of the DMRs listed in Tables 1-8 and/or Tables 12-27 of samples obtained from subjects having NEPC or PRAD are distinguishable. DNA methylation is tissue (and tumor) specific, thus, the DMRs disclosed herein represent a significant advance in biomarkers for use in diagnosing and, in some embodiments, treating a subject having or at risk of developing NEPC or PRAD.

Moreover, the novel approach described herein represents several improvements over the currently available methods. For example, identifying DMRs in tumor tissue, rather than cell-free DNA, allows one to select particular regions of the genome with robust and consistent enrichment of methylation in NEPC compared to PRAD. In contrast, prior approaches to defining DMRs, which utilize a training set of cfDNA samples, are limited by the low and variable content of tumor DNA in patient plasma. Plasma-defined DMRs are likely to be more susceptible to sample-to-sample variation in ctDNA content, with high-ctDNA content samples driving DMR identification. Because DMRs represent a “ground truth,” DNA methylation signals in patient plasma at these regions are more likely to reflect true-positive methylation from NEPC ctDNA than at DMRs defined in plasma. Further, the ability to detect NEPC ctDNA methylation is enhanced by filtering out regions of the genome that are methylated in WBCs, thereby reducing background from WBCs (the major component of cfDNA).

The novel tissue-based approach presented herein improves performance substantially (classification AUROC of 0.88 compared to 0.76 with the standard approach (see Example 2)). The performance of this test is on par with other tests in clinical use, increasing its potential clinical utility compared to prior approaches. This tissue-informed approach can be applied “out of the box”. It does not require analyzing training cfDNA samples and building a model for each new sample that is processed. This feature substantially increases its potential for clinical use.

Additionally, classifiers based on cfDNA methylation are highly susceptible to batch effect because the signal of interest (i.e., presence or absence of ctDNA) is diluted and may be small compared to random sample-to-sample variability. With existing methods, samples must generally be physically processed with training samples to guard against batch effects. The tissue-informed approach described herein overcomes this limitation by not using plasma to identify DMRs. It thereby reduces the risk of batch effect, and for the same reasons, the risk of overfitting DMRs to training data.

Overall, the simplicity of this novel approach—identifying tumor-specific sites of DNA methylation in tumor tissue and counting the number of methylated cfDNA read at these sites—makes it more robust and practical to implement.

The DMRs described herein can comprise between about 10 and about 1000 nucleotides, between about 100 and about 1000 nucleotides, between about 200 and about 1000 nucleotides, between about 300 and about 1000 nucleotides, between about 400 and about 1000 nucleotides, between about 500 and about 1000 nucleotides, between about 600 and about 1000 nucleotides, between about 700 and about 1000 nucleotides, between about 800 and about 1000 nucleotides, or between about 900 and about 1000 nucleotides. In some embodiments, a DMR comprises about 10, 50, 100, 200, or 300 nucleotides.

The genomic loci listed in Tables 1-8 and/or Tables 12-27 (which are also referred to herein as DMRs) can also be used to study the progression of prostate cancer and for the early detection of NEPC in subjects as adenocarcinoma cells trans-differentiate into neuroendocrine prostate cancer cells. In some embodiments, one or more of the genomic loci listed in Tables 1-8 and/or Tables 12-27 is used to detect NEPC in a subject. In some embodiments, the number of genomic loci used to detect NEPC in a subject is about 1, 2, 3, 4, 5, 6, 7, 8, 9, 10, 11, 12, 13, 14, 15, 16, 17, 20, 51, 76, 127, 193, 1,112, or 1,674 genomic loci or more or any range in between, inclusive. For example, in some embodiments, the number of genomic loci used to detect NEPC in a subject is between about 17 and about 20 genomic loci, between about 17 and about 51 genomic loci, between about 17 and about 76 genomic loci, about 17 and about 124 genomic loci, between about 17 and about 193 genomic loci, between about 17 and about 1,112 genomic loci, and/or between about 17 and about 1,674 genomic loci. In some embodiments, the number of genomic loci used to determine if a subject has or is at risk of developing NEPC is between about 51 and about 76 genomic loci, between about 124 and about 193 genomic loci, and/or between about 1,112 and about 1,674 genomic loci.

Tables 1-8 and/or Tables 12-27 disclose the DMRs identified as described in the Examples. These DMRs, or subsets thereof, can be used to identify subjects having or at risk of developing NEPC and/or distinguish NEPC from PRAD.

Lengthy table referenced here
US20240158864A1-20240516-T00001
Please refer to the end of the specification for access instructions.

Lengthy table referenced here
US20240158864A1-20240516-T00002
Please refer to the end of the specification for access instructions.

Lengthy table referenced here
US20240158864A1-20240516-T00003
Please refer to the end of the specification for access instructions.

Lengthy table referenced here
US20240158864A1-20240516-T00004
Please refer to the end of the specification for access instructions.

Lengthy table referenced here
US20240158864A1-20240516-T00005
Please refer to the end of the specification for access instructions.

Lengthy table referenced here
US20240158864A1-20240516-T00006
Please refer to the end of the specification for access instructions.

Lengthy table referenced here
US20240158864A1-20240516-T00007
Please refer to the end of the specification for access instructions.

Lengthy table referenced here
US20240158864A1-20240516-T00008
Please refer to the end of the specification for access instructions.

Lengthy table referenced here
US20240158864A1-20240516-T00009
Please refer to the end of the specification for access instructions.

Lengthy table referenced here
US20240158864A1-20240516-T00010
Please refer to the end of the specification for access instructions.

Lengthy table referenced here
US20240158864A1-20240516-T00011
Please refer to the end of the specification for access instructions.

Lengthy table referenced here
US20240158864A1-20240516-T00012
Please refer to the end of the specification for access instructions.

Lengthy table referenced here
US20240158864A1-20240516-T00013
Please refer to the end of the specification for access instructions.

Lengthy table referenced here
US20240158864A1-20240516-T00014
Please refer to the end of the specification for access instructions.

Lengthy table referenced here
US20240158864A1-20240516-T00015
Please refer to the end of the specification for access instructions.

Lengthy table referenced here
US20240158864A1-20240516-T00016
Please refer to the end of the specification for access instructions.

Lengthy table referenced here
US20240158864A1-20240516-T00017
Please refer to the end of the specification for access instructions.

Lengthy table referenced here
US20240158864A1-20240516-T00018
Please refer to the end of the specification for access instructions.

Lengthy table referenced here
US20240158864A1-20240516-T00019
Please refer to the end of the specification for access instructions.

Lengthy table referenced here
US20240158864A1-20240516-T00020
Please refer to the end of the specification for access instructions.

Lengthy table referenced here
US20240158864A1-20240516-T00021
Please refer to the end of the specification for access instructions.

Lengthy table referenced here
US20240158864A1-20240516-T00022
Please refer to the end of the specification for access instructions.

Lengthy table referenced here
US20240158864A1-20240516-T00023
Please refer to the end of the specification for access instructions.

Lengthy table referenced here
US20240158864A1-20240516-T00024
Please refer to the end of the specification for access instructions.

As shown in Tables 1-4, Tables 13, 15-19, 25 and 26, statistically significant DMRs may be immediately adjacent to each other depending on the detection parameters used to identify the DMRs, and combining these DMRs result in a longer nucleotide sequence that can be evaluated. Conversely, when large DMRs, in some cases, can be divided into small DMR windows. In some embodiments, a subset of DMRs from a larger DMR will retain statistical significance.

In some embodiments, a methylation profile comprising one or more of the DMRs listed in Tables 1-8 and/or Tables 12-27 can be used in combination with other biomarkers to identify a patient having or suspected of having NPEC or PRAD. For example, a methylation profile comprising one or more of the DMRs listed in Tables 1-8 and/or Tables 12-27 and a mutation in a relevant biomarker can be used to identify a subject as having or is at risk of developing NPEC. Relevant biomarkers, such, TP53 and/or RB1, are known in the art. Reliance on only mutational data may lead to inaccurate diagnoses. For example, while mutations in TP53 and/or RB1 are present in over 80% of NEPC subjects, mutations in these genes are also present in over one third of PRAD cases.

The invention provided herein, is also related, in part, to methods of generating an NEPC risk score and using such a risk score to evaluate whether a patient is afflicted with NEPC and/or whether the patient would benefit from platinum based chemotherapy.

Therefore, provided herein are methods of determining if a subject with prostate cancer has or is at risk for developing neuroendocrine prostate cancer (NEPC), the method comprising generating an NEPC Risk Value score for the subject, wherein an NEPC Risk Score of greater than or equal to 0, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, or 0.5 indicates that the subject has or is at risk for developing NEPC.

In some aspects, provided herein are methods of determining if a subject with prostate cancer would benefit from platinum-based chemotherapy, the method comprising generating an NEPC Risk Value score for the subject, wherein an NEPC Risk Score of greater than or equal to 0, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, or 0.5 indicates that the subject would benefit from platinum-based chemotherapy.

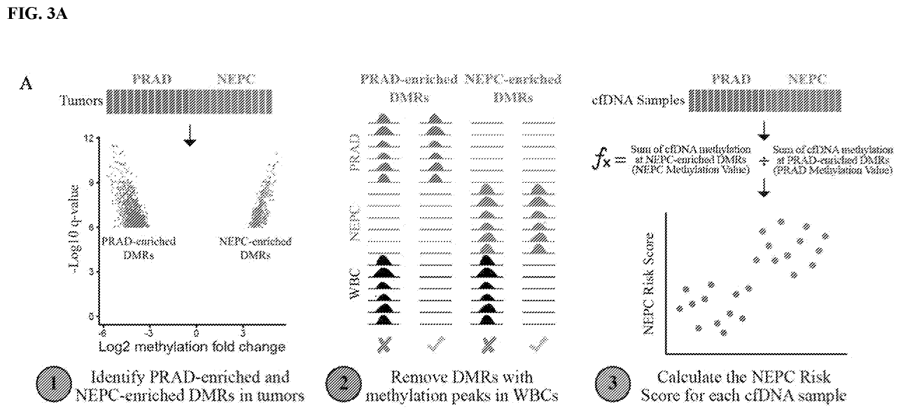

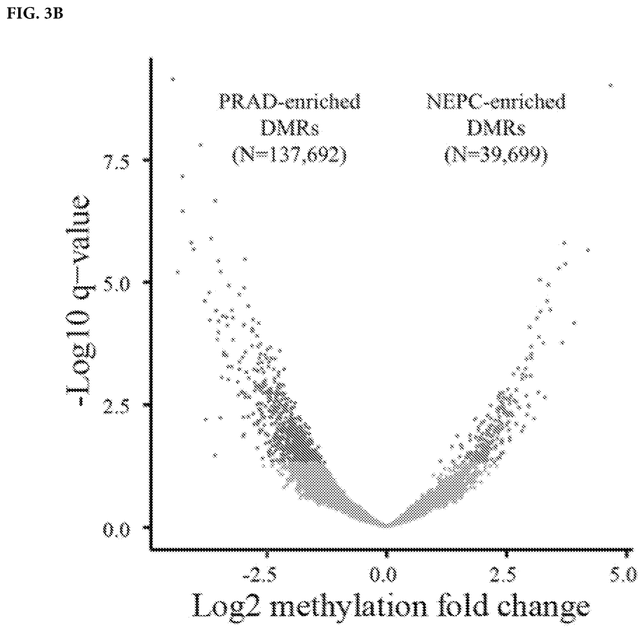

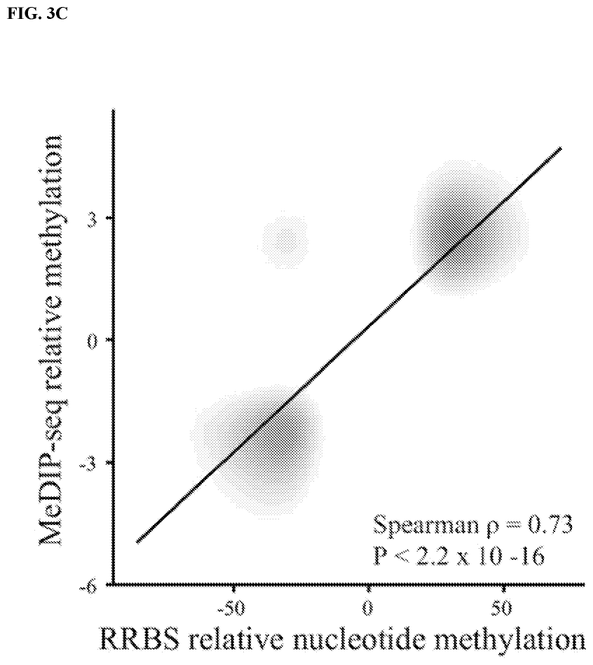

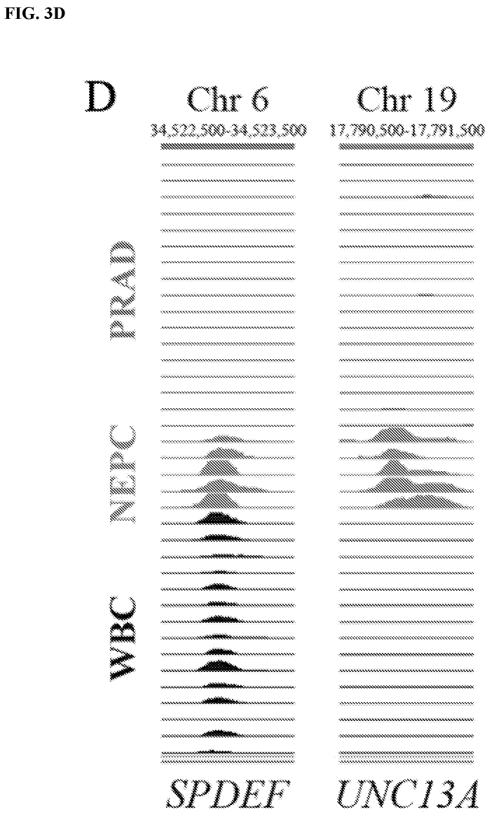

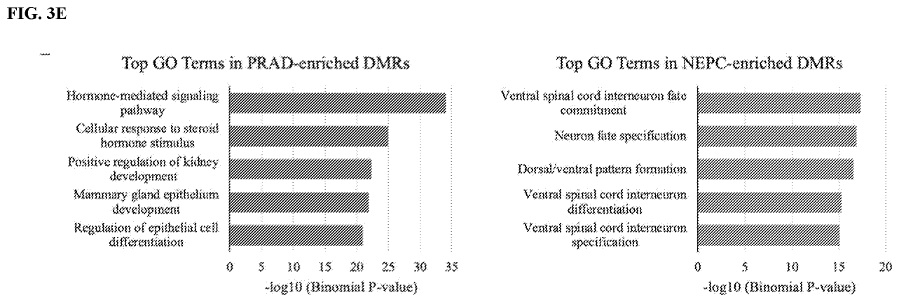

Also provided herein are multiple methods to generate an NEPC Risk score. From cell-free methylated DNA immunoprecipitation sequencing (cfMeDIP-seq) data, a NEPC Methylation Value and PRAD Methylation Value for each sample may calculated by summing the methylated cfDNA fragments at tissue-derived NEPC-enriched and PRAD-enriched DMRs, respectively (FIG. 3A). It should be appreciated that the current methods are not limited to performing cfMeDIP-seq, but can include any method to evaluate the methylation status of genomic loci (e.g., any genomic loci provided herein). It is contemplated that an NEPC Risk Score is calculated for each sample from a patient, and comprises the normalized ratio of the NEPC Methylation Value versus the PRAD Methylation Value.

In some embodiments, the NEPC Risk Value is the log₂ ratio of a NEPC Methylation Value to a PRAD Methylation Value.

In certain embodiments, the NEPC Methylation Value is calculated by summing relative methylation scores of at least two NEPC-enriched differentially methylated regions in DNA from a sample taken from the subject. The NEPC Methylation Value may be calculated by summing relative methylation scores of at least 3, at least 20, at least 76, at least 193, at least 479, at least 504, at least 1674, at least 5552, at least 5604 NEPC-enriched differentially methylated regions in DNA from a sample taken from the subject. The NEPC Methylation Value may be calculated by summing relative methylation scores of at least 2, at least 5, at least 10, at least 15, at least 25, at least 35, at least 45, at least 50, at least 55, at least 65, at least 75, at least 90, at least 100, at least 150, at least 200, at least 250, at least 300, at least 400, at least 500, at least 600, at least 700, at least 800, at least 900, at least 1000, at least 2000, at least 3000, at least 4000, or at least 5000 NEPC-enriched differentially methylated regions in DNA from a sample taken from the subject. The NEPC Methylation Value may be calculated by summing relative methylation scores of any number of NEPC-enriched differentially methylated regions disclosed herein (e.g., in Tables 1-8 and 12-15) in DNA from a sample taken from the subject. Any number of NEPC-enriched differentially methylated regions may be any number from 3 to 6000, for example, the NEPC Methylation Value may be calculated by summing relative methylation scores of at least 1, 6, 11, 16, 21, 26, 31, 36, 41, 46, 51, 56, 61, 66, 71, 76, 81, 86, 91, 96, 101, 106, 111, 116, 121, 126, 131, 136, 141, 146, 151, 156, 161, 166, 171, 176, 181, 186, 191, 196, 201, 206, 211, 216, 221, 226, 231, 236, 241, 246, 251, 256, 261, 266, 271, 276, 281, 286, 291, 296, 301, 306, 311, 316, 321, 326, 331, 336, 341, 346, 351, 356, 361, 366, 371, 376, 381, 386, 391, 396, 401, 406, 411, 416, 421, 426, 431, 436, 441, 446, 451, 456, 461, 466, 471, 476, 481, 486, 491, 496, 501, 506, 511, 516, 521, 526, 531, 536, 541, 546, 551, 556, 561, 566, 571, 576, 581, 586, 591, 596, 601, 606, 611, 616, 621, 626, 631, 636, 641, 646, 651, 656, 661, 666, 671, 676, 681, 686, 691, 696, 701, 706, 711, 716, 721, 726, 731, 736, 741, 746, 751, 756, 761, 766, 771, 776, 781, 786, 791, 796, 801, 806, 811, 816, 821, 826, 831, 836, 841, 846, 851, 856, 861, 866, 871, 876, 881, 886, 891, 896, 901, 906, 911, 916, 921, 926, 931, 936, 941, 946, 951, 956, 961, 966, 971, 976, 981, 986, 991, 996, 1001, 1006, 1011, 1016, 1021, 1026, 1031, 1036, 1041, 1046, 1051, 1056, 1061, 1066, 1071, 1076, 1081, 1086, 1091, 1096, 1101, 1106, 1111, 1116, 1121, 1126, 1131, 1136, 1141, 1146, 1151, 1156, 1161, 1166, 1171, 1176, 1181, 1186, 1191, 1196, 1201, 1206, 1211, 1216, 1221, 1226, 1231, 1236, 1241, 1246, 1251, 1256, 1261, 1266, 1271, 1276, 1281, 1286, 1291, 1296, 1301, 1306, 1311, 1316, 1321, 1326, 1331, 1336, 1341, 1346, 1351, 1356, 1361, 1366, 1371, 1376, 1381, 1386, 1391, 1396, 1401, 1406, 1411, 1416, 1421, 1426, 1431, 1436, 1441, 1446, 1451, 1456, 1461, 1466, 1471, 1476, 1481, 1486, 1491, 1496, 1501, 1506, 1511, 1516, 1521, 1526, 1531, 1536, 1541, 1546, 1551, 1556, 1561, 1566, 1571, 1576, 1581, 1586, 1591, 1596, 1601, 1606, 1611, 1616, 1621, 1626, 1631, 1636, 1641, 1646, 1651, 1656, 1661, 1666, 1671, 1676, 1681, 1686, 1691, 1696, 1701, 1706, 1711, 1716, 1721, 1726, 1731, 1736, 1741, 1746, 1751, 1756, 1761, 1766, 1771, 1776, 1781, 1786, 1791, 1796, 1801, 1806, 1811, 1816, 1821, 1826, 1831, 1836, 1841, 1846, 1851, 1856, 1861, 1866, 1871, 1876, 1881, 1886, 1891, 1896, 1901, 1906, 1911, 1916, 1921, 1926, 1931, 1936, 1941, 1946, 1951, 1956, 1961, 1966, 1971, 1976, 1981, 1986, 1991, 1996, 2001, 2006, 2011, 2016, 2021, 2026, 2031, 2036, 2041, 2046, 2051, 2056, 2061, 2066, 2071, 2076, 2081, 2086, 2091, 2096, 2101, 2106, 2111, 2116, 2121, 2126, 2131, 2136, 2141, 2146, 2151, 2156, 2161, 2166, 2171, 2176, 2181, 2186, 2191, 2196, 2201, 2206, 2211, 2216, 2221, 2226, 2231, 2236, 2241, 2246, 2251, 2256, 2261, 2266, 2271, 2276, 2281, 2286, 2291, 2296, 2301, 2306, 2311, 2316, 2321, 2326, 2331, 2336, 2341, 2346, 2351, 2356, 2361, 2366, 2371, 2376, 2381, 2386, 2391, 2396, 2401, 2406, 2411, 2416, 2421, 2426, 2431, 2436, 2441, 2446, 2451, 2456, 2461, 2466, 2471, 2476, 2481, 2486, 2491, 2496, 2501, 2506, 2511, 2516, 2521, 2526, 2531, 2536, 2541, 2546, 2551, 2556, 2561, 2566, 2571, 2576, 2581, 2586, 2591, 2596, 2601, 2606, 2611, 2616, 2621, 2626, 2631, 2636, 2641, 2646, 2651, 2656, 2661, 2666, 2671, 2676, 2681, 2686, 2691, 2696, 2701, 2706, 2711, 2716, 2721, 2726, 2731, 2736, 2741, 2746, 2751, 2756, 2761, 2766, 2771, 2776, 2781, 2786, 2791, 2796, 2801, 2806, 2811, 2816, 2821, 2826, 2831, 2836, 2841, 2846, 2851, 2856, 2861, 2866, 2871, 2876, 2881, 2886, 2891, 2896, 2901, 2906, 2911, 2916, 2921, 2926, 2931, 2936, 2941, 2946, 2951, 2956, 2961, 2966, 2971, 2976, 2981, 2986, 2991, 2996, 3001, 3006, 3011, 3016, 3021, 3026, 3031, 3036, 3041, 3046, 3051, 3056, 3061, 3066, 3071, 3076, 3081, 3086, 3091, 3096, 3101, 3106, 3111, 3116, 3121, 3126, 3131, 3136, 3141, 3146, 3151, 3156, 3161, 3166, 3171, 3176, 3181, 3186, 3191, 3196, 3201, 3206, 3211, 3216, 3221, 3226, 3231, 3236, 3241, 3246, 3251, 3256, 3261, 3266, 3271, 3276, 3281, 3286, 3291, 3296, 3301, 3306, 3311, 3316, 3321, 3326, 3331, 3336, 3341, 3346, 3351, 3356, 3361, 3366, 3371, 3376, 3381, 3386, 3391, 3396, 3401, 3406, 3411, 3416, 3421, 3426, 3431, 3436, 3441, 3446, 3451, 3456, 3461, 3466, 3471, 3476, 3481, 3486, 3491, 3496, 3501, 3506, 3511, 3516, 3521, 3526, 3531, 3536, 3541, 3546, 3551, 3556, 3561, 3566, 3571, 3576, 3581, 3586, 3591, 3596, 3601, 3606, 3611, 3616, 3621, 3626, 3631, 3636, 3641, 3646, 3651, 3656, 3661, 3666, 3671, 3676, 3681, 3686, 3691, 3696, 3701, 3706, 3711, 3716, 3721, 3726, 3731, 3736, 3741, 3746, 3751, 3756, 3761, 3766, 3771, 3776, 3781, 3786, 3791, 3796, 3801, 3806, 3811, 3816, 3821, 3826, 3831, 3836, 3841, 3846, 3851, 3856, 3861, 3866, 3871, 3876, 3881, 3886, 3891, 3896, 3901, 3906, 3911, 3916, 3921, 3926, 3931, 3936, 3941, 3946, 3951, 3956, 3961, 3966, 3971, 3976, 3981, 3986, 3991, 3996, 4001, 4006, 4011, 4016, 4021, 4026, 4031, 4036, 4041, 4046, 4051, 4056, 4061, 4066, 4071, 4076, 4081, 4086, 4091, 4096, 4101, 4106, 4111, 4116, 4121, 4126, 4131, 4136, 4141, 4146, 4151, 4156, 4161, 4166, 4171, 4176, 4181, 4186, 4191, 4196, 4201, 4206, 4211, 4216, 4221, 4226, 4231, 4236, 4241, 4246, 4251, 4256, 4261, 4266, 4271, 4276, 4281, 4286, 4291, 4296, 4301, 4306, 4311, 4316, 4321, 4326, 4331, 4336, 4341, 4346, 4351, 4356, 4361, 4366, 4371, 4376, 4381, 4386, 4391, 4396, 4401, 4406, 4411, 4416, 4421, 4426, 4431, 4436, 4441, 4446, 4451, 4456, 4461, 4466, 4471, 4476, 4481, 4486, 4491, 4496, 4501, 4506, 4511, 4516, 4521, 4526, 4531, 4536, 4541, 4546, 4551, 4556, 4561, 4566, 4571, 4576, 4581, 4586, 4591, 4596, 4601, 4606, 4611, 4616, 4621, 4626, 4631, 4636, 4641, 4646, 4651, 4656, 4661, 4666, 4671, 4676, 4681, 4686, 4691, 4696, 4701, 4706, 4711, 4716, 4721, 4726, 4731, 4736, 4741, 4746, 4751, 4756, 4761, 4766, 4771, 4776, 4781, 4786, 4791, 4796, 4801, 4806, 4811, 4816, 4821, 4826, 4831, 4836, 4841, 4846, 4851, 4856, 4861, 4866, 4871, 4876, 4881, 4886, 4891, 4896, 4901, 4906, 4911, 4916, 4921, 4926, 4931, 4936, 4941, 4946, 4951, 4956, 4961, 4966, 4971, 4976, 4981, 4986, 4991, 4996, 5001, 5006, 5011, 5016, 5021, 5026, 5031, 5036, 5041, 5046, 5051, 5056, 5061, 5066, 5071, 5076, 5081, 5086, 5091, 5096, 5101, 5106, 5111, 5116, 5121, 5126, 5131, 5136, 5141, 5146, 5151, 5156, 5161, 5166, 5171, 5176, 5181, 5186, 5191, 5196, 5201, 5206, 5211, 5216, 5221, 5226, 5231, 5236, 5241, 5246, 5251, 5256, 5261, 5266, 5271, 5276, 5281, 5286, 5291, 5296, 5301, 5306, 5311, 5316, 5321, 5326, 5331, 5336, 5341, 5346, 5351, 5356, 5361, 5366, 5371, 5376, 5381, 5386, 5391, 5396, 5401, 5406, 5411, 5416, 5421, 5426, 5431, 5436, 5441, 5446, 5451, 5456, 5461, 5466, 5471, 5476, 5481, 5486, 5491, 5496, 5501, 5506, 5511, 5516, 5521, 5526, 5531, 5536, 5541, 5546, 5551, 5556, 5561, 5566, 5571, 5576, 5581, 5586, 5591, 5596, 5601, 5606, 5611, 5616, 5621, 5626, 5631, 5636, 5641, 5646, 5651, 5656, 5661, 5666, 5671, 5676, 5681, 5686, 5691, 5696, 5701, 5706, 5711, 5716, 5721, 5726, 5731, 5736, 5741, 5746, 5751, 5756, 5761, 5766, 5771, 5776, 5781, 5786, 5791, 5796, 5801, 5806, 5811, 5816, 5821, 5826, 5831, 5836, 5841, 5846, 5851, 5856, 5861, 5866, 5871, 5876, 5881, 5886, 5891, 5896, 5901, 5906, 5911, 5916, 5921, 5926, 5931, 5936, 5941, 5946, 5951, 5956, 5961, 5966, 5971, 5976, 5981, 5986, 5991, 5996, or 6000 of NEPC-enriched differentially methylated regions. The number of NEPC-enriched differentially methylated regions to be used in an NEPC risk score may be determined by a log2-fold change cutoff and FDR-adjusted p value cutoff. For example, the log2-fold change cutoff may be 1, 2, 3, 4, or 5. The FDR-adjusted p value cutoff may be 1e-2, 1e-3, 1e-4, 1e-5, 1e-6, 1e-7, 1e-8, or 1e-9.

The NEPC-enriched differentially methylated regions may comprise any one of the genomic loci listed in Tables 1-8 and 12-15, such as any one of the genomic loci listed in Table 3 and/or Table 7.

In some embodiments, the relative methylation scores (rms) are calculated by taking the sum of relative methylation scores at each site, and dividing by the sum of the relative methylation scores across all sites in the genome. In some embodiments, the relative methylation scores are calculated by the R package MEDIPS as described on the World Wide Web at genome.cshlp.org/content/suppl/2010/08/03/gr.110114.110.DC1/Chavez_GR-110114_Supplementary_Methods.pdf and Example 10. In some embodiments, the NEPC Methylation Value is normalized to CpG content of the local sequence.

In some embodiments, the NEPC-enriched differentially methylated regions have a predetermined area under the ROC curve (AUROC) of greater than 0.8, greater than 0.9, greater than 0.95, or greater than 0.99.

In certain embodiments, the PRAD Methylation Value is calculated by summing relative methylation scores of at least two PRAD-enriched differentially methylated regions in DNA from a sample taken from the subject. The PRAD Methylation Value may be calculated by summing relative methylation scores of at least 14, at least 42, at least 100, at least 277, at least 783, at least 1600, at least 2347, at least 7287, at least 21688, or at least 26209 of the PRAD-enriched differentially methylated regions in DNA from a sample taken from the subject. The PRAD Methylation Value may be calculated by summing relative methylation scores of at least 10, at least 25, at least 50, at least 75, at least 100, at least 125, at least 150, at least 175, at least 200, at least 225, at least 250, at least 300, at least 500, at least 800, at least 1000, at least 2000, at least 3000, at least 4000, at least 5000, at least 6000, at least 7000, at least 8000, at least 9000, at least 10000, at least 15000, at least 20000, at least 25000, or at least 30000 of the PRAD-enriched differentially methylated regions in DNA from a sample taken from the subject. The PRAD Methylation Value may be calculated by summing relative methylation scores of any number of PRAD-enriched differentially methylated regions disclosed herein (e.g., in Tables 16-27) in DNA from a sample taken from the subject. Any number of PRAD-enriched differentially methylated regions may be any number from 10 to 30000, for example, the PRAD Methylation Value may be calculated by summing relative methylation scores of 1, 26, 51, 76, 101, 126, 151, 176, 201, 226, 251, 276, 301, 326, 351, 376, 401, 426, 451, 476, 501, 526, 551, 576, 601, 626, 651, 676, 701, 726, 751, 776, 801, 826, 851, 876, 901, 926, 951, 976, 1001, 1026, 1051, 1076, 1101, 1126, 1151, 1176, 1201, 1226, 1251, 1276, 1301, 1326, 1351, 1376, 1401, 1426, 1451, 1476, 1501, 1526, 1551, 1576, 1601, 1626, 1651, 1676, 1701, 1726, 1751, 1776, 1801, 1826, 1851, 1876, 1901, 1926, 1951, 1976, 2001, 2026, 2051, 2076, 2101, 2126, 2151, 2176, 2201, 2226, 2251, 2276, 2301, 2326, 2351, 2376, 2401, 2426, 2451, 2476, 2501, 2526, 2551, 2576, 2601, 2626, 2651, 2676, 2701, 2726, 2751, 2776, 2801, 2826, 2851, 2876, 2901, 2926, 2951, 2976, 3001, 3026, 3051, 3076, 3101, 3126, 3151, 3176, 3201, 3226, 3251, 3276, 3301, 3326, 3351, 3376, 3401, 3426, 3451, 3476, 3501, 3526, 3551, 3576, 3601, 3626, 3651, 3676, 3701, 3726, 3751, 3776, 3801, 3826, 3851, 3876, 3901, 3926, 3951, 3976, 4001, 4026, 4051, 4076, 4101, 4126, 4151, 4176, 4201, 4226, 4251, 4276, 4301, 4326, 4351, 4376, 4401, 4426, 4451, 4476, 4501, 4526, 4551, 4576, 4601, 4626, 4651, 4676, 4701, 4726, 4751, 4776, 4801, 4826, 4851, 4876, 4901, 4926, 4951, 4976, 5001, 5026, 5051, 5076, 5101, 5126, 5151, 5176, 5201, 5226, 5251, 5276, 5301, 5326, 5351, 5376, 5401, 5426, 5451, 5476, 5501, 5526, 5551, 5576, 5601, 5626, 5651, 5676, 5701, 5726, 5751, 5776, 5801, 5826, 5851, 5876, 5901, 5926, 5951, 5976, 6001, 6026, 6051, 6076, 6101, 6126, 6151, 6176, 6201, 6226, 6251, 6276, 6301, 6326, 6351, 6376, 6401, 6426, 6451, 6476, 6501, 6526, 6551, 6576, 6601, 6626, 6651, 6676, 6701, 6726, 6751, 6776, 6801, 6826, 6851, 6876, 6901, 6926, 6951, 6976, 7001, 7026, 7051, 7076, 7101, 7126, 7151, 7176, 7201, 7226, 7251, 7276, 7301, 7326, 7351, 7376, 7401, 7426, 7451, 7476, 7501, 7526, 7551, 7576, 7601, 7626, 7651, 7676, 7701, 7726, 7751, 7776, 7801, 7826, 7851, 7876, 7901, 7926, 7951, 7976, 8001, 8026, 8051, 8076, 8101, 8126, 8151, 8176, 8201, 8226, 8251, 8276, 8301, 8326, 8351, 8376, 8401, 8426, 8451, 8476, 8501, 8526, 8551, 8576, 8601, 8626, 8651, 8676, 8701, 8726, 8751, 8776, 8801, 8826, 8851, 8876, 8901, 8926, 8951, 8976, 9001, 9026, 9051, 9076, 9101, 9126, 9151, 9176, 9201, 9226, 9251, 9276, 9301, 9326, 9351, 9376, 9401, 9426, 9451, 9476, 9501, 9526, 9551, 9576, 9601, 9626, 9651, 9676, 9701, 9726, 9751, 9776, 9801, 9826, 9851, 9876, 9901, 9926, 9951, 9976, 10001, 10026, 10051, 10076, 10101, 10126, 10151, 10176, 10201, 10226, 10251, 10276, 10301, 10326, 10351, 10376, 10401, 10426, 10451, 10476, 10501, 10526, 10551, 10576, 10601, 10626, 10651, 10676, 10701, 10726, 10751, 10776, 10801, 10826, 10851, 10876, 10901, 10926, 10951, 10976, 11001, 11026, 11051, 11076, 11101, 11126, 11151, 11176, 11201, 11226, 11251, 11276, 11301, 11326, 11351, 11376, 11401, 11426, 11451, 11476, 11501, 11526, 11551, 11576, 11601, 11626, 11651, 11676, 11701, 11726, 11751, 11776, 11801, 11826, 11851, 11876, 11901, 11926, 11951, 11976, 12001, 12026, 12051, 12076, 12101, 12126, 12151, 12176, 12201, 12226, 12251, 12276, 12301, 12326, 12351, 12376, 12401, 12426, 12451, 12476, 12501, 12526, 12551, 12576, 12601, 12626, 12651, 12676, 12701, 12726, 12751, 12776, 12801, 12826, 12851, 12876, 12901, 12926, 12951, 12976, 13001, 13026, 13051, 13076, 13101, 13126, 13151, 13176, 13201, 13226, 13251, 13276, 13301, 13326, 13351, 13376, 13401, 13426, 13451, 13476, 13501, 13526, 13551, 13576, 13601, 13626, 13651, 13676, 13701, 13726, 13751, 13776, 13801, 13826, 13851, 13876, 13901, 13926, 13951, 13976, 14001, 14026, 14051, 14076, 14101, 14126, 14151, 14176, 14201, 14226, 14251, 14276, 14301, 14326, 14351, 14376, 14401, 14426, 14451, 14476, 14501, 14526, 14551, 14576, 14601, 14626, 14651, 14676, 14701, 14726, 14751, 14776, 14801, 14826, 14851, 14876, 14901, 14926, 14951, 14976, 15001, 15026, 15051, 15076, 15101, 15126, 15151, 15176, 15201, 15226, 15251, 15276, 15301, 15326, 15351, 15376, 15401, 15426, 15451, 15476, 15501, 15526, 15551, 15576, 15601, 15626, 15651, 15676, 15701, 15726, 15751, 15776, 15801, 15826, 15851, 15876, 15901, 15926, 15951, 15976, 16001, 16026, 16051, 16076, 16101, 16126, 16151, 16176, 16201, 16226, 16251, 16276, 16301, 16326, 16351, 16376, 16401, 16426, 16451, 16476, 16501, 16526, 16551, 16576, 16601, 16626, 16651, 16676, 16701, 16726, 16751, 16776, 16801, 16826, 16851, 16876, 16901, 16926, 16951, 16976, 17001, 17026, 17051, 17076, 17101, 17126, 17151, 17176, 17201, 17226, 17251, 17276, 17301, 17326, 17351, 17376, 17401, 17426, 17451, 17476, 17501, 17526, 17551, 17576, 17601, 17626, 17651, 17676, 17701, 17726, 17751, 17776, 17801, 17826, 17851, 17876, 17901, 17926, 17951, 17976, 18001, 18026, 18051, 18076, 18101, 18126, 18151, 18176, 18201, 18226, 18251, 18276, 18301, 18326, 18351, 18376, 18401, 18426, 18451, 18476, 18501, 18526, 18551, 18576, 18601, 18626, 18651, 18676, 18701, 18726, 18751, 18776, 18801, 18826, 18851, 18876, 18901, 18926, 18951, 18976, 19001, 19026, 19051, 19076, 19101, 19126, 19151, 19176, 19201, 19226, 19251, 19276, 19301, 19326, 19351, 19376, 19401, 19426, 19451, 19476, 19501, 19526, 19551, 19576, 19601, 19626, 19651, 19676, 19701, 19726, 19751, 19776, 19801, 19826, 19851, 19876, 19901, 19926, 19951, 19976, 20001, 20026, 20051, 20076, 20101, 20126, 20151, 20176, 20201, 20226, 20251, 20276, 20301, 20326, 20351, 20376, 20401, 20426, 20451, 20476, 20501, 20526, 20551, 20576, 20601, 20626, 20651, 20676, 20701, 20726, 20751, 20776, 20801, 20826, 20851, 20876, 20901, 20926, 20951, 20976, 21001, 21026, 21051, 21076, 21101, 21126, 21151, 21176, 21201, 21226, 21251, 21276, 21301, 21326, 21351, 21376, 21401, 21426, 21451, 21476, 21501, 21526, 21551, 21576, 21601, 21626, 21651, 21676, 21701, 21726, 21751, 21776, 21801, 21826, 21851, 21876, 21901, 21926, 21951, 21976, 22001, 22026, 22051, 22076, 22101, 22126, 22151, 22176, 22201, 22226, 22251, 22276, 22301, 22326, 22351, 22376, 22401, 22426, 22451, 22476, 22501, 22526, 22551, 22576, 22601, 22626, 22651, 22676, 22701, 22726, 22751, 22776, 22801, 22826, 22851, 22876, 22901, 22926, 22951, 22976, 23001, 23026, 23051, 23076, 23101, 23126, 23151, 23176, 23201, 23226, 23251, 23276, 23301, 23326, 23351, 23376, 23401, 23426, 23451, 23476, 23501, 23526, 23551, 23576, 23601, 23626, 23651, 23676, 23701, 23726, 23751, 23776, 23801, 23826, 23851, 23876, 23901, 23926, 23951, 23976, 24001, 24026, 24051, 24076, 24101, 24126, 24151, 24176, 24201, 24226, 24251, 24276, 24301, 24326, 24351, 24376, 24401, 24426, 24451, 24476, 24501, 24526, 24551, 24576, 24601, 24626, 24651, 24676, 24701, 24726, 24751, 24776, 24801, 24826, 24851, 24876, 24901, 24926, 24951, 24976, 25001, 25026, 25051, 25076, 25101, 25126, 25151, 25176, 25201, 25226, 25251, 25276, 25301, 25326, 25351, 25376, 25401, 25426, 25451, 25476, 25501, 25526, 25551, 25576, 25601, 25626, 25651, 25676, 25701, 25726, 25751, 25776, 25801, 25826, 25851, 25876, 25901, 25926, 25951, 25976, 26001, 26026, 26051, 26076, 26101, 26126, 26151, 26176, 26201, 26226, 26251, 26276, 26301, 26326, 26351, 26376, 26401, 26426, 26451, 26476, 26501, 26526, 26551, 26576, 26601, 26626, 26651, 26676, 26701, 26726, 26751, 26776, 26801, 26826, 26851, 26876, 26901, 26926, 26951, 26976, 27001, 27026, 27051, 27076, 27101, 27126, 27151, 27176, 27201, 27226, 27251, 27276, 27301, 27326, 27351, 27376, 27401, 27426, 27451, 27476, 27501, 27526, 27551, 27576, 27601, 27626, 27651, 27676, 27701, 27726, 27751, 27776, 27801, 27826, 27851, 27876, 27901, 27926, 27951, 27976, 28001, 28026, 28051, 28076, 28101, 28126, 28151, 28176, 28201, 28226, 28251, 28276, 28301, 28326, 28351, 28376, 28401, 28426, 28451, 28476, 28501, 28526, 28551, 28576, 28601, 28626, 28651, 28676, 28701, 28726, 28751, 28776, 28801, 28826, 28851, 28876, 28901, 28926, 28951, 28976, 29001, 29026, 29051, 29076, 29101, 29126, 29151, 29176, 29201, 29226, 29251, 29276, 29301, 29326, 29351, 29376, 29401, 29426, 29451, 29476, 29501, 29526, 29551, 29576, 29601, 29626, 29651, 29676, 29701, 29726, 29751, 29776, 29801, 29826, 29851, 29876, 29901, 29926, 29951, or 29976 of PRAD-enriched differentially methylated regions. The number of PRAD-enriched differentially methylated regions to be used in an NEPC risk score may be determined by a log2-fold change cutoff and FDR-adjusted p value cutoff. For example, the log2-fold change cutoff may be 1, 2, 3, 4, or 5. The FDR-adjusted p value cutoff may be 1e-2, 1e-3, 1e-4, 1e-5, 1e-6, 1e-7, 1e-8, or 1e-9.

The PRAD-enriched differentially methylated regions may comprise any one of the genomic loci listed in Tables 16-27, such as any one of the genomic loci listed in Table 17 and/or Table 21.

PRAD-enriched differentially methylated regions in DNA from a sample taken from the subject. In some embodiments, the relative methylation scores (rms) are calculated by taking the sum of relative methylation scores at each site, and dividing by the sum of relative methylation scores across all sites in the genome. In some embodiments, the relative methylation scores are calculated by the R package MEDIPS as described on the World Wide Web at genome.cshlp.org/content/suppl/2010/08/03/gr.110114.110.DC1/Chavez_GR-110114_Supplementary_Methods.pdf and Example 10. In some embodiments, the PRAD Methylation Value is normalized to CpG content of the local sequence.

The PRAD-enriched differentially methylated regions may comprise the genomic loci listed in Tables 16-27. In some embodiments, the PRAD-enriched differentially methylated regions have a predetermined area under the ROC curve (AUROC) of greater than 0.8, greater than 0.9, greater than 0.95, or greater than 0.99.

It should be appreciated that the number of sites used, the log₂-fold change cutoff and/or FDR-adjusted p value cutoff can be varied to alter the sensitivity of diagnostics and methods described herein.

In some embodiments, a kit for determining if a subject with prostate cancer has or is at risk for developing neuroendocrine prostate cancer (NEPC), the kit comprising a reagent for detecting the presence, absence, or level of methylation in the genomic DNA or cell free DNA (cfDNA) in a sample, or circulating tumor DNA (ctDNA) wherein the methylation profile comprises one or more of the genomic loci listed in any one of the Tables disclosed herein (i.e., Tables 1-8 and Tables 12-27). The method may further comprise calculating a NEPC Risk Score as described herein.

In some embodiments, a kit for determining if a subject with prostate cancer would benefit from platinum-based chemotherapy, the kit comprising a reagent for detecting the presence, absence, or level of methylation in the genomic DNA or cell free DNA (cfDNA) in a sample, or circulating tumor DNA (ctDNA) wherein the methylation profile comprises one or more of the genomic loci listed in any one of the Tables disclosed herein (i.e., Tables 1-8 and Tables 12-27). The method may further comprise calculating a NEPC Risk Score as described herein.

### I. Definitions

The articles “a” and “an” are used herein to refer to one or to more than one (i.e. to at least one) of the grammatical object of the article. By way of example, “an element” means one element or more than one element.

The term “altered amount” or “altered level” refers to increased or decreased level of methylation of one or more genomic loci (e.g., the DMRs listed in Tables 1-8 and/or Tables 12-27) in a cancer sample, as compared to the methylation level in a control sample. The term “altered amount” of methylation can be used to refer to hypermethylation or hypomethylation of a genomic locus.

The level of methylation of genomic loci (e.g., the DMRs listed in Tables 1-8 and/or Tables 12-27) in a subject is “significantly” higher or lower than the normal amount of the methylation at these loci, if the amount of the methylation is greater or less, respectively, than the normal level by an amount greater than the standard error of the assay employed to assess amount, and preferably at least 10%, 20%, 30%, 40%, 50%, 60%, 70%, 80%, 90%, 100%, 150%, 200%, 300%, 350%, 400%, 500%, 600%, 700%, 800%, 900%, 1000% or than that amount. Alternately, the level of methylation of the biomarker in the subject can be considered “significantly” higher or lower than the normal level of methylation if the level is at least about two, and preferably at least about three, four, or five times, higher or lower, respectively, than the normal level of methylation. Such “significance” can also be applied to any other measured parameter described herein, such as for expression, inhibition, cytotoxicity, cell growth, and the like.

Unless otherwise specified here within, the terms “antibody” and “antibodies” refers to antigen-binding portions adaptable to be expressed within cells as “intracellular antibodies.” (Chen et al. (1994) Human Gene Ther. 5:595-601). Methods are well-known in the art for adapting antibodies to target (e.g., inhibit) intracellular moieties, such as the use of single-chain antibodies (scFvs), modification of immunoglobulin VL domains for hyperstability, modification of antibodies to resist the reducing intracellular environment, generating fusion proteins that increase intracellular stability and/or modulate intracellular localization, and the like. Intracellular antibodies can also be introduced and expressed in one or more cells, tissues or organs of a multicellular organism, for example for prophylactic and/or therapeutic purposes (e.g., as a gene therapy) (see, at least PCT Pubis. WO 08/020079, WO 94/02610, WO 95/22618, and WO 03/014960; U.S. Pat. No. 7,004,940; Cattaneo and Biocca (1997) Intracellular Antibodies: Development and Applications (Landes and Springer-Verlag publs.); Kontermann (2004) Methods 34:163-170; Cohen et al. (1998) Oncogene 17:2445-2456; Auf der Maur et al. (2001) FEBS Lett. 508:407-412; Shaki-Loewenstein et al. (2005) J. Immunol. Meth. 303:19-39).

Antibodies may be polyclonal or monoclonal; xenogeneic, allogeneic, or syngeneic; or modified forms thereof (e g humanized, chimeric, etc.). Antibodies may also be fully human. Preferably, antibodies of the present invention bind specifically or substantially specifically to a biomarker polypeptide or fragment thereof. The terms “monoclonal antibodies” and “monoclonal antibody composition”, as used herein, refer to a population of antibody polypeptides that contain only one species of an antigen binding site capable of immunoreacting with a particular epitope of an antigen, whereas the term “polyclonal antibodies” and “polyclonal antibody composition” refer to a population of antibody polypeptides that contain multiple species of antigen binding sites capable of interacting with a particular antigen. A monoclonal antibody composition typically displays a single binding affinity for a particular antigen with which it immunoreacts.

Antibodies may also be “humanized,” which is intended to include antibodies made by a non-human cell having variable and constant regions that have been altered to resemble more closely antibodies that would be made by a human cell. For example, by altering the non-human antibody amino acid sequence to incorporate amino acids found in human germline immunoglobulin sequences. The humanized antibodies of the present invention may include amino acid residues not encoded by human germline immunoglobulin sequences (e.g., mutations introduced by random or site-specific mutagenesis in vitro or by somatic mutation in vivo), for example in the CDRs. The term “humanized antibody,” as used herein, also includes antibodies in which CDR sequences derived from the germline of another mammalian species, such as a mouse, have been grafted onto human framework sequences.

A “blocking” antibody or an antibody “antagonist” is one that inhibits or reduces at least one biological activity of the antigen(s) it binds. In certain embodiments, the blocking antibodies or antagonist antibodies or fragments thereof described herein substantially or completely inhibit a given biological activity of the antigen(s).

The term “biomarker” refers to a measurable entity that can be used in determining if a subject has or is at risk of developing prostate cancer (i.e., NEPC). Biomarkers can include, without limitation, nucleic acids and proteins. As described herein, any relevant characteristic of a biomarker can be used, such as the copy number, amount, activity, location, modification (e.g., phosphorylation), genomic alterations (e.g., deletion, gain, or mutation), epigenetic alterations (e.g., hypermethylation or hypomethylation) and the like.

The term “body fluid” refers to fluids that are excreted or secreted from the body as well as fluids that are normally not (e g amniotic fluid, aqueous humor, bile, blood and blood plasma, cerebrospinal fluid, cerumen and earwax, Cowper's fluid or pre-ejaculatory fluid, chyle, chyme, stool, interstitial fluid, intracellular fluid, lymph, menses, mucus, pleural fluid, pus, saliva, sebum, semen, serum, sweat, synovial fluid, tears, urine, vitreous humor, vomit).

The terms “cancer,” “tumor,” and “hyperproliferative” refer to the presence of cells possessing characteristics typical of cancer-causing cells, such as uncontrolled proliferation, immortality, metastatic potential, rapid growth and proliferation rate, and certain characteristic morphological features. Unless otherwise stated, the terms include metaplasias.

Cancer cells can make up a tumor as well as circulate within the blood stream of an animal Cancerous tumors may shed cells that or cellular debris that can be isolated from the blood or tissue sample. For example, a cancerous tumor may shed dead cells, which upon degradation of the cellular and nuclear membranes release interior cellular component (e.g. DNA) into the extracellular environment. A cancer cell can be a non-tumorigenic cancer cell, such as a leukemia cell. As used herein, the term “cancer” includes premalignant as well as malignant cancers. In certain embodiments, “cancer” refers to prostate cancer. Prostate cancer (Pca) is one of the most common types of cancer in men. Prostate cancer is often a slow growing cancer, which when confined to the prostate gland, often does not cause serious harm. However, some types are aggressive and can spread quickly (i.e., metastasize) to other organs or tissues of the body. Prostate cancer can be diagnosed with a digital rectal exam and/or prostate specific antigen (PSA) screening. An elevated serum PSA level can indicate the presence of prostate cancer. PSA is used as a marker for prostate cancer because it is secreted only by prostate cells. When PSA or digital tests indicate a strong likelihood that cancer is present, a transrectal ultrasound (TRUS) is used to map the prostate and show any suspicious areas. Biopsies of various sectors of the prostate are used to determine if prostate cancer is present.

Treatment options depend on the stage of the prostate cancer. Men with a 10-year life expectancy or less who have a low Gleason number and whose tumor has not spread beyond the prostate are often treated with watchful waiting (no treatment). Treatment options for more aggressive cancers include surgical treatments, such as radical prostatectomy (RP) in which the prostate is completely removed (with or without nerve sparing techniques), and radiation, applied through an external beam that directs the dose to the prostate from outside the body or via low-dose radioactive seeds that are implanted within the prostate to kill cancer cells locally. Anti-androgen hormone therapy may also be used, alone or in conjunction with surgery or radiation. Hormone therapy may use luteinizing hormone-releasing hormones (LH-RH) analogs, which block the pituitary from producing hormones that stimulate testosterone production. Patients may need to have injections of LH-RH analogs for the rest of their lives. While surgical and hormonal treatments are often effective for localized prostate cancer, advanced disease remains essentially incurable. Androgen ablation is the most common therapy for advanced prostate cancer, leading to massive apoptosis of androgen-dependent malignant cells and temporary tumor regression. However, the tumor may reemerge and can proliferate independent of androgen signals.

“Castrate-resistant prostate cancer (CRPC)” is a form of prostate cancer characterized by disease progression despite androgen-deprivation therapy (ADT) and may present as one or any combination of a continuous rise in serum levels of prostate-specific antigen (PSA), progression of pre-existing disease, or appearance of new metastases.

Hormonal therapy that targets the androgen receptor is a common treatment for prostate cancer patients (including castration-resistant prostate cancer) with metastatic spread. For example, enzalutamide and abiraterone are potent AR-targeted therapies approved for treating CRPC. Other treatment methods include, but are not limited to, alternative hormone therapies, taxane chemotherapy (e.g., docetaxel, cabazitaxel), bone-targeting radiopharmaceuticals (e.g., radium-223) and immunotherapy (e.g., sipuleucel-T), etc., with the goals of prolonging survival, minimizing complications, and maintaining quality of life.

Castration-resistant prostate cancer can be histologically characterized as prostate adenocarcinomas (PRAD) or neuroendocrine prostate cancer (NEPC). Histologically, NEPC is characterized by, and can be distinguished from PRAD by, the presence of neuroendocrine carcinoma cells that do not express androgen receptor or secrete prostate specific antigen (PSA). NEPC cells usually express neuroendocrine markers such as chromogranin A, synaptophysin, and neuron-specific enolase. (Wang et al. (2008) Am. J. Surg. Pathol. 32:65-71).

Prostate adenocarcinoma (PRAD) cells can trans-differentiate to NEPC cells as a resistance mechanism to potent androgen receptor signaling inhibitors (ARSIs). NEPC emerges in up to 1 in 6 men with metastatic prostate cancer and is associated with poor responsiveness to ARSIs and shorter survival. In contrast, men with NEPC are more likely to respond to platinum-based chemotherapy, highlighting the clinical and therapeutic importance of detecting this resistance phenotype.

The term “coding region” refers to regions of a nucleotide sequence comprising codons which are translated into amino acid residues, whereas the term “noncoding region” refers to regions of a nucleotide sequence that are not translated into amino acids (e.g., 5′ and 3′ untranslated regions).

The term “diagnosing cancer” includes the use of the methods, systems, and code of the present invention to determine the presence or absence of a cancer or subtype thereof in an individual. The term also includes methods, systems, and code for assessing the level of disease activity in an individual.

“Homologous” as used herein, refers to nucleotide sequence similarity between two regions of the same nucleic acid strand or between regions of two different nucleic acid strands. When a nucleotide residue position in both regions is occupied by the same nucleotide residue, then the regions are homologous at that position. A first region is homologous to a second region if at least one nucleotide residue position of each region is occupied by the same residue. Homology between two regions is expressed in terms of the proportion of nucleotide residue positions of the two regions that are occupied by the same nucleotide residue. By way of example, a region having the nucleotide sequence 5′-ATTGCC-3′ and a region having the nucleotide sequence 5′-TATGGC-3′ share 50% homology. Preferably, the first region comprises a first portion and the second region comprises a second portion, whereby, at least about 50%, and preferably at least about 75%, at least about 90%, or at least about 95% of the nucleotide residue positions of each of the portions are occupied by the same nucleotide residue. More preferably, all nucleotide residue positions of each of the portions are occupied by the same nucleotide residue.

The term “immunotherapy” or “immunotherapies” refer to any treatment that uses certain parts of a subject's immune system to fight diseases such as cancer. The subject's own immune system is stimulated (or suppressed), with or without administration of one or more agent for that purpose Immunotherapies that are designed to elicit or amplify an immune response are referred to as “activation immunotherapies” Immunotherapies that are designed to reduce or suppress an immune response are referred to as “suppression immunotherapies.” Any agent believed to have an immune system effect on the genetically modified transplanted cancer cells can be assayed to determine whether the agent is an immunotherapy and the effect that a given genetic modification has on the modulation of immune response. In some embodiments, the immunotherapy is cancer cell-specific. In some embodiments, immunotherapy can be “untargeted,” which refers to administration of agents that do not selectively interact with immune system cells, yet modulates immune system function. Representative examples of untargeted therapies include, without limitation, chemotherapy, gene therapy, and radiation therapy.

Immunotherapy is one form of targeted therapy that may comprise, for example, the use of cancer vaccines and/or sensitized antigen presenting cells. For example, an oncolytic virus is a virus that is able to infect and lyse cancer cells, while leaving normal cells unharmed, making them potentially useful in cancer therapy. Replication of oncolytic viruses both facilitates tumor cell destruction and produces dose amplification at the tumor site. They may also act as vectors for anticancer genes, allowing them to be specifically delivered to the tumor site. The immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). For example, anti-VEGF and mTOR inhibitors are known to be effective in treating renal cell carcinoma. Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumor or cancer.

Immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen) Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumor or cancer.

In some embodiments, the immunotherapy described herein comprises at least one of immunogenic chemotherapies. The term “immunogenic chemotherapy” refers to any chemotherapy that has been demonstrated to induce immunogenic cell death, a state that is detectable by the release of one or more damage-associated molecular pattern (DAMP) molecules, including, but not limited to, calreticulin, ATP and HMGB1 (Kroemer et al. (2013) Annu. Rev. Immunol. 31:51-72). Specific representative examples of consensus immunogenic chemotherapies include anthracyclines, such as doxorubicin and the platinum drug, oxaliplatin, 5′-fluorouracil, among others.

In some embodiments, immunotherapy comprises inhibitors of one or more immune checkpoints. The term “immune checkpoint” refers to a group of molecules on the cell surface of CD4+ and/or CD8+ T cells that by down-modulate or inhibit an anti-tumor immune response Immune checkpoint proteins are well-known in the art and include, without limitation, CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRP, CD47, CD48, 2B4 (CD244), B7.1, B7.2, ILT-2, ILT-4, TIGIT, HHLA2, butyrophilins, IDO, CD39, CD73 and A2aR (see, for example, WO 2012/177624). The term further encompasses biologically active protein fragment, as well as nucleic acids encoding full-length immune checkpoint proteins and biologically active protein fragments thereof. In some embodiment, the term further encompasses any fragment according to homology descriptions provided herein. In one embodiment, the immune checkpoint is PD-1.

Immune checkpoints and their sequences are well known in the art and representative embodiments are described below. For example, the term “PD-1” refers to a member of the immunoglobulin gene superfamily that functions as a coinhibitory receptor having PD-L1 and PD-L2 as known ligands. PD-1 was previously identified using a subtraction cloning based approach to select for genes upregulated during TCR-induced activated T cell death. PD-1 is a member of the CD28/CTLA-4 family of molecules based on its ability to bind to PD-L1. Like CTLA-4, PD-1 is rapidly induced on the surface of T-cells in response to anti-CD3 (Agata et al. 25 (1996) Int. Immunol. 8:765). In contrast to CTLA-4, however, PD-1 is also induced on the surface of B-cells (in response to anti-IgM). PD-1 is also expressed on a subset of thymocytes and myeloid cells (Agata et al. (1996) supra; Nishimura et al. (1996) Int. Immunol. 8:773).

“Anti-immune checkpoint therapy” refers to the use of agents that inhibit immune checkpoint nucleic acids and/or proteins. Inhibition of one or more immune checkpoints can block or otherwise neutralize inhibitory signaling to thereby upregulate an immune response in order to more efficaciously treat cancer. Exemplary agents useful for inhibiting immune checkpoints include antibodies, small molecules, peptides, peptidomimetics, natural ligands, and derivatives of natural ligands, that can either bind and/or inactivate or inhibit immune checkpoint proteins, or fragments thereof; as well as RNA interference, antisense, nucleic acid aptamers, etc. that can downregulate the expression and/or activity of immune checkpoint nucleic acids, or fragments thereof. Exemplary agents for upregulating an immune response include antibodies against one or more immune checkpoint proteins block the interaction between the proteins and its natural receptor(s); a non-activating form of one or more immune checkpoint proteins (e.g., a dominant negative polypeptide); small molecules or peptides that block the interaction between one or more immune checkpoint proteins and its natural receptor(s); fusion proteins (e.g. the extracellular portion of an immune checkpoint inhibition protein fused to the Fc portion of an antibody or immunoglobulin) that bind to its natural receptor(s); nucleic acid molecules that block immune checkpoint nucleic acid transcription or translation; and the like. Such agents can directly block the interaction between the one or more immune checkpoints and its natural receptor(s) (e.g., antibodies) to prevent inhibitory signaling and upregulate an immune response. Alternatively, agents can indirectly block the interaction between one or more immune checkpoint proteins and its natural receptor(s) to prevent inhibitory signaling and upregulate an immune response.

For example, a soluble version of an immune checkpoint protein ligand such as a stabilized extracellular domain can binding to its receptor to indirectly reduce the effective concentration of the receptor to bind to an appropriate ligand. In some embodiments, anti-PD-1 antibodies, anti-PD-L1 antibodies, and/or anti-PD-L2 antibodies, either alone or in combination, are used to inhibit immune checkpoints. These embodiments are also applicable to specific therapy against particular immune checkpoints, such as the PD-1 pathway (e.g., anti-PD-1 pathway therapy, otherwise known as PD-1 pathway inhibitor therapy).

The term “androgen receptor-directed therapy” refers to any therapy that targets androgen receptor signaling in a subject in need thereof. For example, used for treating prostate cancer by targeting androgen receptor (AR)-signaling in the subject. The therapy may act through inhibition of androgen synthesis or through AR targeting directly. Androgen receptor (AR)-directed therapies include, but are not limited to abiraterone or enzalutamide.

The term “methylation” refers to an epigenetic modification on the genetic material (e.g., genomic DNA) of a cell or a subject. Specifically, DNA methylation is a chemical modification of DNA performed by enzymes called methyltransferases, in which a methyl group (m) is added to certain cytosines (C) of DNA. This non-mutational (epigenetic) process (mC) is a critical factor in gene expression regulation. DNA methylation plays an important role in gene expression. By turning genes off that are not needed, DNA methylation is an essential control mechanism for the organism development and function. Alternatively, abnormal DNA methylation is one of the mechanisms involved with the development of many cancers.

DNA methylation often occurs at clustered CpG dinucleotides, or CpG islands, in promoter regions. CpG islands are short sequences rich in the CpG dinucleotide, and can be found in the 5′ region of about half of all human genes. Methylation of these promoter regions can result in transcriptional inactivation of the affected genes. Aberrant methylation of CpG islands has been detected in genetic diseases such as the fragile-X syndrome, in aging cells, and in neoplasia. About half of the tumor suppressor genes that have been shown to be mutated in the germline of patients with familial cancer syndromes have also been shown to be aberrantly methylated in some proportion of sporadic cancers, including Rb, VHL, p16, hMLH1, and BRCA1. Methylation of tumor suppressor genes in cancer is usually associated with (1) lack of gene transcription and (2) absence of coding region mutation. Thus, CpG island methylation can serve as a mechanism of gene inactivation in cancer.

The term “hypermethylation” refers to an increase in the epigenetic methylation of cytosine and adenosine residues in DNA from a sample compared to a control. The term “hypomethylation” refers to a decrease in the epigenetic methylation of cytosine and adenosine residues in DNA from a sample compared to a control. The term “hypomethylation” refers to an decrease in the epigenetic methylation of cytosine and adenosine residues in DNA from a sample compared to a control. The term “hypomethylation” refers to a decrease in the epigenetic methylation of cytosine and adenosine residues in DNA from a sample compared to a control In certain embodiments, the control is a site-specific tissue-based threshold that discriminates between PRAD and NEPC tissue samples. Such thresholds can be determined using methods described in Example 1. In some embodiments, the control is the site-specific tissue-based methylation level determined NEPC or PRAD samples.

In some embodiments, hypermethylation or hypomethylation refers to a level of methylation at a locus that is greater than or less than, respectively, the normal amount of the methylation at the locus. In some embodiments, hypermethalation or hypomethylation refers to an amount or level of methylation that is greater or less, respectively, than the normal level (e.g., the level of methylation of the locus in a normal control) by an amount greater than the standard error of the assay employed to assess amount, and preferably at least 10%, 20%, 30%, 40%, 50%, 60%, 70%, 80%, 90%, 100%, 150%, 200%, 300%, 350%, 400%, 500%, 600%, 700%, 800%, 900%, 1000% or than that amount. Alternately, the level of methylation of the biomarker in the subject can be considered hypermethylation or hypomethylation if the level is at least about two, and preferably at least about three, four, or five times, higher or lower, respectively, than the normal level of methylation.

As used herein, the term “NEPC Risk Score” is a value based on evaluating methylation differences between samples. In some embodiments, the CpG-normalized relative methylation scores are calculated across 300 bp windows for a cfDNA sample (Lienhard M. (2014) Bioinforma Oxf Engl.; 30:284-6; Pelizzola M, et al. (2008) Genome Res.; 18:1652-9) relative methylation scores are summed in cfDNA at NEPC-enriched PDX DMRs for each sample and normalized to the sum of relative methylation scores (rms) values across all 300 bp windows. A nucleic acid bp length window of at least 100, 125, 150, 175, 200, 225, 250, 275, 300, 325, 350, 375, 400, 425, 450, 475, 500, or more, or any range in between, inclusive, such as 100-500, 200-400, 250-350 bp, and the like, may be used. Relative methylation scores (rms) scores may be calculated by taking the sum of rms scores at each site, and dividing by the sum of rms scores across all sites in the genome. This value is termed “NEPC Methylation Value.” The same process may be performed for PRAD-enriched PDX DMRs to derive a “PRAD Methylation Value.” The log₂ ratio of the NEPC Methylation Value to the PRAD Methylation Value is calculated and these values may be normalized to the median score in cfDNA from cancer-free controls (at least one, at least two, at least three, at least five, at least ten or at least 15 cancer-free controls). This value is termed the “NEPC Risk Score.” Any method provided herein may include calculating an NEPC Risk Score as described herein. For example, any method described herein which includes determining the hypo or hypermethylation status of any one of the genomic loci and/or a DMR listed in a Tables herein may include calculating an NEPC Risk Score.

The term “immune response” includes T cell mediated and/or B cell mediated immune responses. Exemplary immune responses include T cell responses, e.g., cytokine production and cellular cytotoxicity. In addition, the term immune response includes immune responses that are indirectly effected by T cell activation, e.g., antibody production (humoral responses) and activation of cytokine responsive cells, e.g., macrophages.

The term “immunotherapeutic agent” can include any molecule, peptide, antibody or other agent that can stimulate a host immune system to generate an immune response to a tumor or cancer in the subject. Various immunotherapeutic agents are useful in the compositions and methods described herein.

The term “inhibit” includes the decrease, limitation, or blockage of, for example, a particular action, function, or interaction. In some embodiments, a cancer (e.g., NEPC) is “inhibited” if at least one symptom of the cancer is alleviated, terminated, slowed, or prevented. As used herein, cancer is also “inhibited” if recurrence or metastasis of the cancer is reduced, slowed, delayed, or prevented.

The term “interaction,” when referring to an interaction between two molecules, refers to the physical contact (e.g., binding) of the molecules with one another. Generally, such an interaction results in an activity (which produces a biological effect) of one or both of said molecules.

A “kit” is any manufacture (e.g., a package or container) comprising at least one reagent, e.g., a probe or small molecule, for specifically detecting and/or affecting the expression of a marker of the present invention. The kit may be promoted, distributed, or sold as a unit for performing the methods of the present invention. The kit may comprise one or more reagents necessary to express a composition useful in the methods of the present invention. In certain embodiments, the kit may further comprise a reference standard, e.g., a nucleic acid encoding a protein that does not affect or regulate signaling pathways controlling cell growth, division, migration, survival, or apoptosis. One skilled in the art can envision many such control proteins, including, but not limited to, common molecular tags (e.g., green fluorescent protein and beta-galactosidase), proteins not classified in any of pathway encompassing cell growth, division, migration, survival or apoptosis by Gene Ontology reference, or ubiquitous housekeeping proteins. Reagents in the kit may be provided in individual containers or as mixtures of two or more reagents in a single container. In addition, instructional materials that describe the use of the compositions within the kit can be included.

The term “neoadjuvant therapy” refers to a treatment given before the primary treatment. Examples of neoadjuvant therapy can include chemotherapy, radiation therapy, and hormone therapy. For example, in treating breast cancer, neoadjuvant therapy can allows patients with large breast cancer to undergo breast-conserving surgery.

The “normal” level of expression of a biomarker is the level of expression of the biomarker in cells of a subject, e.g., a human patient, not afflicted with a cancer. An “over-expression” or “significantly higher level of expression” of a biomarker refers to an expression level in a test sample that is greater than the standard error of the assay employed to assess expression, and is preferably at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more higher than the expression activity or level of the biomarker in a control sample (e.g., sample from a healthy subject not having the biomarker associated disease) and preferably, the average expression level of the biomarker in several control samples. A “significantly lower level of expression” of a biomarker refers to an expression level in a test sample that is at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more lower than the expression level of the biomarker in a control sample (e.g., sample from a healthy subject not having the biomarker associated disease) and preferably, the average expression level of the biomarker in several control samples.

An “over-expression” or “significantly higher level of expression” of a biomarker refers to an expression level in a test sample that is greater than the standard error of the assay employed to assess expression, and is preferably at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more higher than the expression activity or level of the biomarker in a control sample (e.g., sample from a healthy subject not having the biomarker associated disease) and preferably, the average expression level of the biomarker in several control samples. A “significantly lower level of expression” of a biomarker refers to an expression level in a test sample that is at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more lower than the expression level of the biomarker in a control sample (e.g., sample from a healthy subject not having the biomarker associated disease) and preferably, the average expression level of the biomarker in several control samples.

The term “predictive” or “predictive assay” refers to methods or assays described herein that predict if a subject has or is at risk of developing NEPC. For example, a predictive assay described herein includes the use of methylation status e.g., hyper- or hypomethylation of a genomic loci (e.g., the genomic loci listed in Tables 1-8 and/or Tables 12-27) for determining the likelihood of response of a cancer to an anti-cancer therapy. Such predictive use of the methylation of genomic loci may be confirmed by, e.g., (1) increased or decreased copy number (e.g., by FISH, FISH plus SKY, single-molecule sequencing, e.g., as described in the art at least at J. Biotechnol., 86:289-301, or qPCR), overexpression or underexpression of a biomarker nucleic acid (e.g., by ISH, Northern Blot, or qPCR), increased or decreased biomarker protein (e.g., by IHC), or increased or decreased activity, e.g., in more than about 5%, 6%, 7%, 8%, 9%, 10%, 11%, 12%, 13%, 14%, 15%, 20%, 25%, 30%, 40%, 50%, 60%, 70%, 80%, 90%, 95%, 100%, or more of assayed human cancers types or cancer samples; (2) its absolute or relatively modulated presence or absence in a biological sample, e.g., a sample containing tissue, whole blood, serum, plasma, buccal scrape, saliva, cerebrospinal fluid, urine, stool, or bone marrow, from a subject, e.g. a human, afflicted with cancer; (3) its absolute or relatively modulated presence or absence in clinical subset of patients with cancer.

A predictive test should have a sufficient specificity and sensitivity. A receiver operating characteristic (ROC) cureve is a plot of the true positive rate and false positive rate of an assay. A ROC analysis can be used to select a threshold that best distinguishes one subpopulation (i.e., NEPC subjects) from another subpopulation (e.g., healthy controls or PRAD subjects). False positives occur when a subject tests positive but does not actually have the disease. False negatives occur when a subject tests negative, but they are actually positive for a trait (i.e., NEPC). To plot the ROC curve, the True Positive Rate (TPR) and False Positive Rate (FPR) can be measured, while the decision threshold can be continuously varied. A perfect test will have an area under the ROC curve of 1.0; the random test will have an area of 0.5. The threshold is selected to provide an acceptable level of specificity and sensitivity. For example, the area under a ROC curve (AUROC) score greater than 0.70 can be an acceptable level of specificity and sensitivity. Higher AUROC scores indicate greater levels of specificity and sensitivity. Thus, AUROC scores between 0.7 and 1.0 are preferred. In some embodiments, the AUROC score is 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or even 1.0.

P-values can also be used to as a measure of the reliability of a predictive test. P-values measure the probability that an observation could happen by chance. The lower the p-value, the less likely the observation happened by chance. In some embodiments, p-values less than 0.05 are considered statistically significant. In some embodiments, p-values less than 0.01 are considered statistically significant.

The terms “prevent,” “preventing,” “prevention,” “prophylactic treatment,” and the like refer to reducing the probability of developing a disease, disorder, or condition in a subject, who does not have, but is at risk of or susceptible to developing a disease, disorder, or condition.

The term “prognosis” includes a prediction of the probable course and outcome of cancer or the likelihood of recovery from the disease. In some embodiments, the use of statistical algorithms provides a prognosis of cancer in an individual. For example, the prognosis can be surgery, development of a clinical subtype of cancer (e.g., solid tumors, such as esophageal cancer and gastric cancer), development of one or more clinical factors, or recovery from the disease.

The term “response to an anti-cancer therapy” relates to any response of the hyperproliferative disorder (e.g., cancer) to an anti-cancer agent, preferably to a change in tumor mass and/or volume after initiation of neoadjuvant or adjuvant therapy. Hyperproliferative disorder response may be assessed, for example for efficacy or in a neoadjuvant or adjuvant situation, where the size of a tumor after systemic intervention can be compared to the initial size and dimensions as measured by CT, PET, mammogram, ultrasound or palpation. Responses may also be assessed by caliper measurement or pathological examination of the tumor after biopsy or surgical resection. Response may be recorded in a quantitative fashion like percentage change in tumor volume or in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment of hyperproliferative disorder response may be done early after the onset of neoadjuvant or adjuvant therapy, e.g., after a few hours, days, weeks or preferably after a few months. A typical endpoint for response assessment is upon termination of neoadjuvant chemotherapy or upon surgical removal of residual tumor cells and/or the tumor bed. This is typically three months after initiation of neoadjuvant therapy. In some embodiments, clinical efficacy of the therapeutic treatments described herein may be determined by measuring the clinical benefit rate (CBR). The clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR=CR+PR+SD over 6 months. In some embodiments, the CBR for a particular cancer therapeutic regimen is at least 25%, 30%, 35%, 40%, 45%, 50%, 55%, 60%, 65%, 70%, 75%, 80%, 85%, or more. Additional criteria for evaluating the response to cancer therapies are related to “survival,” which includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g., time of diagnosis or start of treatment) and end point (e.g., death, recurrence or metastasis). In addition, criteria for efficacy of treatment can be expanded to include response to chemotherapy, probability of survival, probability of metastasis within a given time period, and probability of tumor recurrence. For example, in order to determine appropriate threshold values, a particular cancer therapeutic regimen can be administered to a population of subjects and the outcome can be correlated to biomarker measurements that were determined prior to administration of any cancer therapy. The outcome measurement may be pathologic response to therapy given in the neoadjuvant setting. Alternatively, outcome measures, such as overall survival and disease-free survival can be monitored over a period of time for subjects following cancer therapy for which biomarker measurement values are known. In certain embodiments, the doses administered are standard doses known in the art for cancer therapeutic agents. The period of time for which subjects are monitored can vary. For example, subjects may be monitored for at least 2, 4, 6, 8, 10, 12, 14, 16, 18, 20, 25, 30, 35, 40, 45, 50, 55, or 60 months. Biomarker measurement threshold values that correlate to outcome of a cancer therapy can be determined using well-known methods in the art, such as those described in the Examples section.

The term “resistance” refers to an acquired or natural resistance of a cancer sample or a mammal to a cancer therapy (i.e., being nonresponsive to or having reduced or limited response to the therapeutic treatment), such as having a reduced response to a therapeutic treatment by 25% or more, for example, 30%, 40%, 50%, 60%, 70%, 80%, or more, to 2-fold, 3-fold, 4-fold, 5-fold, 10-fold, 15-fold, 20-fold or more. The reduction in response can be measured by comparing with the same cancer sample or mammal before the resistance is acquired, or by comparing with a different cancer sample or a mammal that is known to have no resistance to the therapeutic treatment. A typical acquired resistance to chemotherapy is called “multidrug resistance.” The multidrug resistance can be mediated by P-glycoprotein or can be mediated by other mechanisms, or it can occur when a mammal is infected with a multi-drug-resistant microorganism or a combination of microorganisms. The determination of resistance to a therapeutic treatment is routine in the art and within the skill of an ordinarily skilled clinician, for example, can be measured by cell proliferative assays and cell death assays as described herein as “sensitizing.” In some embodiments, the term “reverses resistance” means that the use of a second agent in combination with a primary cancer therapy (e.g., chemotherapeutic or radiation therapy) is able to produce a significant decrease in tumor volume at a level of statistical significance (e.g., p<0.05) when compared to tumor volume of untreated tumor in the circumstance where the primary cancer therapy (e.g., chemotherapeutic or radiation therapy) alone is unable to produce a statistically significant decrease in tumor volume compared to tumor volume of untreated tumor. This generally applies to tumor volume measurements made at a time when the untreated tumor is growing log rhythmically.

The terms “response” or “responsiveness” refers to an anti-cancer response, e.g. in the sense of reduction of tumor size or inhibiting tumor growth. The terms can also refer to an improved prognosis, for example, as reflected by an increased time to recurrence, which is the period to first recurrence censoring for second primary cancer as a first event or death without evidence of recurrence, or an increased overall survival, which is the period from treatment to death from any cause. To respond or to have a response means there is a beneficial endpoint attained when exposed to a stimulus. Alternatively, a negative or detrimental symptom is minimized, mitigated or attenuated on exposure to a stimulus. It will be appreciated that evaluating the likelihood that a tumor or subject will exhibit a favorable response is equivalent to evaluating the likelihood that the tumor or subject will not exhibit favorable response (i.e., will exhibit a lack of response or be non-responsive).

An “RNA interfering agent” as used herein, is defined as any agent that interferes with or inhibits expression of a target biomarker gene by RNA interference (RNAi). Such RNA interfering agents include, but are not limited to, nucleic acid molecules including RNA molecules that are homologous to the target biomarker gene of the present invention, or a fragment thereof, short interfering RNA (siRNA), and small molecules which interfere with or inhibit expression of a target biomarker nucleic acid by RNA interference (RNAi).

“RNA interference (RNAi)” is an evolutionally conserved process whereby the expression or introduction of RNA of a sequence that is identical or highly similar to a target biomarker nucleic acid results in the sequence specific degradation or specific post-transcriptional gene silencing (PTGS) of messenger RNA (mRNA) transcribed from that targeted gene (see Coburn and Cullen (2002) J. Virol. 76:9225), thereby inhibiting expression of the target biomarker nucleic acid. In one embodiment, the RNA is double stranded RNA (dsRNA). This process has been described in plants, invertebrates, and mammalian cells. In nature, RNAi is initiated by the dsRNA-specific endonuclease Dicer, which promotes processive cleavage of long dsRNA into double-stranded fragments termed siRNAs. siRNAs are incorporated into a protein complex that recognizes and cleaves target mRNAs. RNAi can also be initiated by introducing nucleic acid molecules, e.g., synthetic siRNAs or RNA interfering agents, to inhibit or silence the expression of target biomarker nucleic acids. As used herein, “inhibition of target biomarker nucleic acid expression” or “inhibition of marker gene expression” includes any decrease in expression or protein activity or level of the target biomarker nucleic acid or protein encoded by the target biomarker nucleic acid. The decrease may be of at least 30%, 40%, 50%, 60%, 70%, 80%, 90%, 95% or 99% or more as compared to the expression of a target biomarker nucleic acid or the activity or level of the protein encoded by a target biomarker nucleic acid that has not been targeted by an RNA interfering agent.

The term “sample” used for detecting or determining the presence or level of at least one biomarker is typically brain tissue, cerebrospinal fluid, whole blood, plasma, serum, saliva, urine, stool (e.g., feces), tears, and any other bodily fluid (e.g., as described above under the definition of “body fluids”), or a tissue sample (e.g., biopsy) such as a small intestine, colon sample, or surgical resection tissue. In certain instances, the method of the present invention further comprises obtaining the sample from the individual prior to detecting or determining the presence or level of at least one marker in the sample.

“Short interfering RNA” (siRNA), also referred to herein as “small interfering RNA” is defined as an agent which functions to inhibit expression of a target biomarker nucleic acid, e.g., by RNAi. An siRNA may be chemically synthesized, may be produced by in vitro transcription, or may be produced within a host cell. In one embodiment, siRNA is a double stranded RNA (dsRNA) molecule of about 15 to about 40 nucleotides in length, preferably about 15 to about 28 nucleotides, more preferably about 19 to about 25 nucleotides in length, and more preferably about 19, 20, 21, or 22 nucleotides in length, and may contain a 3′ and/or 5′ overhang on each strand having a length of about 0, 1, 2, 3, 4, or 5 nucleotides. The length of the overhang is independent between the two strands, i.e., the length of the overhang on one strand is not dependent on the length of the overhang on the second strand. Preferably, the siRNA is capable of promoting RNA interference through degradation or specific post-transcriptional gene silencing (PTGS) of the target messenger RNA (mRNA).

In another embodiment, an siRNA is a small hairpin (also called stem loop) RNA (shRNA). In one embodiment, these shRNAs are composed of a short (e.g., 19-25 nucleotide) antisense strand, followed by a 5-9 nucleotide loop, and the analogous sense strand. Alternatively, the sense strand may precede the nucleotide loop structure and the antisense strand may follow. These shRNAs may be contained in plasmids, retroviruses, and lentiviruses and expressed from, for example, the pol III U6 promoter, or another promoter (see, e.g., Stewart et al. (2003) RNA 9(4):493-501).

RNA interfering agents, e.g., siRNA molecules, may be administered to a patient having or at risk for having cancer, to inhibit expression of a biomarker gene which is overexpressed in cancer and thereby treat, prevent, or inhibit cancer in the subject.

The term “small molecule” is a term of the art and includes molecules that are less than about 1000 molecular weight or less than about 500 molecular weight. In one embodiment, small molecules do not exclusively comprise peptide bonds. In another embodiment, small molecules are not oligomeric. Exemplary small molecule compounds which can be screened for activity include, but are not limited to, peptides, peptidomimetics, nucleic acids, carbohydrates, small organic molecules (e.g., polyketides) (Cane et al. (1998) Science 282:63), and natural product extract libraries. In another embodiment, the compounds are small, organic non-peptidic compounds. In a further embodiment, a small molecule is not biosynthetic.

The term “specific binding” refers to antibody binding to a predetermined antigen. Typically, the antibody binds with an affinity (K_{D}) of approximately less than 10⁻⁷ M, such as approximately less than 10⁻⁸ M, 10⁻⁹ M or 10⁻¹⁰ M or even lower when determined by surface plasmon resonance (SPR) technology in a BIACORE® assay instrument using an antigen of interest as the analyte and the antibody as the ligand, and binds to the predetermined antigen with an affinity that is at least 1.1-, 1.2-, 1.3-, 1.4-, 1.5-, 1.6-, 1.7-, 1.8-, 1.9-, 2.0-, 2.5-, 3.0-, 3.5-, 4.0-, 4.5-, 5.0-, 6.0-, 7.0-, 8.0-, 9.0-, or 10.0-fold or greater than its affinity for binding to a non-specific antigen (e.g., BSA, casein) other than the predetermined antigen or a closely-related antigen. The phrases “an antibody recognizing an antigen” and “an antibody specific for an antigen” are used interchangeably herein with the term “an antibody which binds specifically to an antigen.” Selective binding is a relative term referring to the ability of an antibody to discriminate the binding of one antigen over another.

The term “subject” refers to any healthy animal, mammal or human, or any animal, mammal or human afflicted with a cancer, e.g., brain, lung, ovarian, pancreatic, liver, breast, prostate, and/or colorectal cancers, melanoma, multiple myeloma, and the like. The term “subject” is interchangeable with “patient.”

The term “survival” includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g. time of diagnosis or start of treatment) and end point (e.g. death, recurrence or metastasis). In addition, criteria for efficacy of treatment can be expanded to include response to chemotherapy, probability of survival, probability of metastasis within a given time period, and probability of tumor recurrence.

The term “therapeutic effect” refers to a local or systemic effect in animals, particularly mammals, and more particularly humans, caused by a pharmacologically active substance. The term thus means any substance intended for use in the diagnosis, cure, mitigation, treatment or prevention of disease or in the enhancement of desirable physical or mental development and conditions in an animal or human. The phrase “therapeutically-effective amount” means that amount of such a substance that produces some desired local or systemic effect at a reasonable benefit/risk ratio applicable to any treatment. In certain embodiments, a therapeutically effective amount of a compound will depend on its therapeutic index, solubility, and the like. For example, certain compounds discovered by the methods of the present invention may be administered in a sufficient amount to produce a reasonable benefit/risk ratio applicable to such treatment.

The terms “therapeutically-effective amount” and “effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment. Toxicity and therapeutic efficacy of subject compounds may be determined by standard pharmaceutical procedures in cell cultures or experimental animals, e.g., for determining the LD₅₀) and the ED₅₀. Compositions that exhibit large therapeutic indices are preferred. In some embodiments, the LD₅₀ (lethal dosage) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more reduced for the agent relative to no administration of the agent. Similarly, the ED₅₀ (i.e., the concentration which achieves a half-maximal inhibition of symptoms) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more increased for the agent relative to no administration of the agent. Also, Similarly, the IC₅₀ (i.e., the concentration which achieves half-maximal cytotoxic or cytostatic effect on cancer cells) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more increased for the agent relative to no administration of the agent. In some embodiments, cancer cell growth in an assay can be inhibited by at least about 10%, 15%, 20%, 25%, 30%, 35%, 40%, 45%, 50%, 55%, 60%, 65%, 70%, 75%, 80%, 85%, 90%, 95%, or even 100%. In another embodiment, at least about a 10%, 15%, 20%, 25%, 30%, 35%, 40%, 45%, 50%, 55%, 60%, 65%, 70%, 75%, 80%, 85%, 90%, 95%, or even 100% decrease in a solid malignancy can be achieved.

There is a known and definite correspondence between the amino acid sequence of a particular protein and the nucleotide sequences that can code for the protein, as defined by the genetic code (shown below). Likewise, there is a known and definite correspondence between the nucleotide sequence of a particular nucleic acid and the amino acid sequence encoded by that nucleic acid, as defined by the genetic code.

| GENETIC CODE | |
|---|---|
| Alanine (Ala, A) | GCA, GCC, GCG, GCT |
| Arginine (Arg, R) | AGA, ACG, CGA, |
| | CGC, CGG, CGT |
| Asparagine (Asn, N) | AAC, AAT |
| Aspartic acid (Asp, D) | GAC, GAT |
| Cysteine (Cys, C) | TGC, TGT |
| Glutamic acid (Glu, E) | GAA, GAG |
| Glutamine (Gln, Q) | CAA, CAG |
| Glycine (Gly, G) | GGA, GGC, GGG, GGT |
| Histidine (His, H) | CAC, CAT |
| Isoleucine (Ile, I) | ATA, ATC, ATT |
| Leucine (Leu, L) | CTA, CTC, CTG, |
| | CTT, TTA, TTG |
| Lysine (Lys, K) | AAA, AAG |
| Methionine (Met, M) | ATG |
| Phenylalanine (Phe, F) | TTC, TTT |
| Proline (Pro, P) | CCA, CCC, CCG, CCT |
| Serine (Ser, S) | AGC, AGT, TCA, |
| | TCC, TCG, TCT |
| Threonine (Thr, T) | ACA, ACC, ACG, ACT |
| Tryptophan (Trp, W) | TGG |
| Tyrosine (Tyr, Y) | TAC, TAT |
| Valine (Val, V) | GTA, GTC, GTG, GTT |
| Termination signal (end) | TAA, TAG, TGA |

An important and well-known feature of the genetic code is its redundancy, whereby, for most of the amino acids used to make proteins, more than one coding nucleotide triplet may be employed (illustrated above). Therefore, a number of different nucleotide sequences may code for a given amino acid sequence. Such nucleotide sequences are considered functionally equivalent since they result in the production of the same amino acid sequence in all organisms (although certain organisms may translate some sequences more efficiently than they do others). Moreover, occasionally, a methylated variant of a purine or pyrimidine may be found in a given nucleotide sequence. Such methylations do not affect the coding relationship between the trinucleotide codon and the corresponding amino acid.

In view of the foregoing, the nucleotide sequence of a DNA or RNA encoding a biomarker nucleic acid (or any portion thereof) can be used to derive the polypeptide amino acid sequence, using the genetic code to translate the DNA or RNA into an amino acid sequence. Likewise, for polypeptide amino acid sequence, corresponding nucleotide sequences that can encode the polypeptide can be deduced from the genetic code (which, because of its redundancy, will produce multiple nucleic acid sequences for any given amino acid sequence). Thus, description and/or disclosure herein of a nucleotide sequence that encodes a polypeptide should be considered to also include description and/or disclosure of the amino acid sequence encoded by the nucleotide sequence. Similarly, description and/or disclosure of a polypeptide amino acid sequence herein should be considered to also include description and/or disclosure of all possible nucleotide sequences that can encode the amino acid sequence.

### II. Subjects

The present invention involves determining if a subject has or is at risk of developing neuroendocrine prostate cancer (NEPC). A subject can be a mammal including a human or a non-human mammal (e.g., mouse, rat, primate, domestic animal, such as a dog, cat, cow, horse, and the like). In some embodiments, the subject is an animal model of prostate cancer. For example, the animal model can comprise a xenograft of a human-derived prostate cancer.

In some embodiments, the subject has NEPC. The subject may be resistant to androgen receptor (AR)-based therapies. In other embodiments, the subject is responsive to AR-based therapies. The subject may not have undergone previous treatments for prostate cancer generally or NEPC specifically. Such treatments include, but are not limited to, chemotherapy, radiation therapy, targeted therapy, and/or immunotherapies. In other embodiments, the subject has undergone previous treatments for prostate cancer, such as the treatments recited in this disclosure. In certain embodiments, the subject has had surgery to remove cancerous or precancerous tissue. In other embodiments, the cancerous tissue has not been removed, e.g., the cancerous tissue may be located in an inoperable region of the body, such as in a tissue that is essential for life, or in a region where a surgical procedure would cause considerable risk of harm to the patient.

### III. Sample Collection, Preparation and Separation

In some embodiments, the methylation of genomic loci (e.g., hyper and/or hypomethylation) in a sample is compared to a control. The sample can be from a subject, such as a human subject having, suspected of having, or at risk of developing neuroendocrine prostate cancer. A subject sample can be, for example, a tissue sample or a bodily fluid sample. In some embodiments, the sample is a tumor biopsy or a liquid biopsy. In certain embodiments, the sample comprises genomic DNA (gDNA), cell-free DNA (cfDNA), or circulating tumor DNA (ctDNA). Reagents and protocols for obtaining and analyzing cfDNA and ctDNA, such as circulating in the blood stream or other tissue, are commercially available as described in the Examples and also well-known in the art (see, for example, Anker et al. (1999) Cancer and Metastasis Rev. 18:65-73; Wua et al. (2002) Clin. Chim. Acta 321:77-87; Fiegl et al. (2005) Cancer Res. 15:1141; Pathak et al. (2006) Clin. Chem. 52:1833-1842; Schwarzenbach et al. (2009) Clin. Cancer Res. 15:1032; Schwarzenbach et al. (2011) Nat. Rev. Cancer 11:426-437).

In some embodiments, the control is a predetermined reference value that can be compared to data generated from the subject sample. In some embodiments, the control is a control sample. A control sample can be from the same subject or from a different subject. The control sample is typically a normal, non-diseased sample. However, in some embodiments, such as for staging of disease or for evaluating the efficacy of treatment, the control sample can be from a diseased tissue In some embodiments, the control sample can be from a subject having prostate adenocarcinoma (PRAD). In some embodiments, the level of methylation at differentially methylated regions (DMRs, e.g., those in Tables 1-8 and/or Tables 12-27) is compared to a pre-determined level. The predetermined level can be obtained from normal samples, from samples derived from a subject with NEPC, or a sample derived from a subject with PRAD. In some embodiments, the methylation profile of a set of DMRs is compared to the methylation profile of a control sample. As described herein, “pre-determined” methylation levels for one or more DMRs may be used to define a methylation profile that can be used diagnose a subject as having or at risk for developing NEPC. A pre-determined methylation level may be determined in populations of patients with or without cancer. The pre-determined methylation level for a particular DMR can be a single number, equally applicable to every patient, or the pre-determined methylation level can vary according to specific subpopulations of patients. Age, weight, height, and other factors of a subject may affect the pre-determined methylation level of a DMR in the individual. Furthermore, the pre-determined methylation level of a DMR and/or the methylation profile can be determined for each subject individually. In one embodiment, the methylation level of a DMR or a methylation profile determined and/or compared in a method described herein are based on absolute measurements.

In another embodiment, the levels of methylation determined and/or compared in a method described herein are based on relative measurements, such as ratios (e.g., methylation level before a treatment relative to after a treatment, such measurements relative to a spiked or man-made control, such measurements relative to the methylation of a housekeeping gene, and the like). For example, the relative analysis can be based on the ratio of pre-treatment methylation measurement as compared to post-treatment methylation measurement. Pre-treatment methylation measurement can be made at any time prior to initiation of anti-cancer therapy. Post-treatment methylation measurement can be made at any time after initiation of anti-cancer therapy. In some embodiments, post-treatment methylation measurements are made 1, 2, 3, 4, 5, 6, 7, 8, 9, 10, 11, 12, 13, 14, 15, 16, 17, 18, 19, 20 weeks or more after initiation of anti-cancer therapy, and even longer toward indefinitely for continued monitoring. Treatment can comprise anti-cancer therapy, such as a platinum-based chemotherapy alone or in combination with other anti-cancer agents, such as with AR-targeted therapies and/or immunotherapies.

A predetermined methylation measurement can be any suitable standard. For example, the predetermined methylation measurement can be obtained from the same or a different human for whom a patient selection is being assessed. In one embodiment, the predetermined methylation measurement can be obtained from a previous assessment of the same patient. In such a manner, the progress of the selection of the patient can be monitored over time. In addition, the control can be obtained from an assessment of another human or multiple humans, e.g., selected groups of humans, if the subject is a human. In such a manner, the extent of the selection of the human for whom selection is being assessed can be compared to suitable other humans, e.g., other humans who are in a similar situation to the human of interest, such as those suffering from similar or the same condition(s) and/or of the same ethnic group.

In some embodiments of the present invention the change in methylation of a DMR from the pre-determined level is about 0.1, 0.2, 0.3, 0.4, 0.5, 0.6, 0.7, 0.8, 0.9, 1.0, 1.5, 2.0, 2.5, 3.0, 3.5, 4.0, 4.5, or 5.0 fold or greater, or any range in between, inclusive. Such cutoff values apply equally when the measurement is based on relative changes, such as based on the ratio of pre-treatment biomarker measurement as compared to post-treatment biomarker measurement.

Biological samples can be collected from a variety of sources from a patient including a body fluid sample, cell sample, or a tissue sample comprising nucleic acids and/or proteins. “Body fluids” refer to fluids that are excreted or secreted from the body as well as fluids that are normally not (e.g., blood and blood plasma, Cowper's fluid or pre-ejaculatory fluid, chyle, chyme, stool, interstitial fluid, intracellular fluid, lymph, menses, saliva, sebum, semen, serum, sweat, synovial fluid, tears, urine, vitreous humor, vomit). In a preferred embodiment, the subject and/or control sample is selected from the group consisting of cells, cell lines, histological slides, paraffin embedded tissues, biopsies, whole blood, serum, plasma, buccal scrape, saliva, cerebrospinal fluid, urine, stool, and bone marrow. In one embodiment, the sample is serum, plasma, or urine. In another embodiment, the sample is serum.

Samples are collected from individuals repeatedly over a period of time (e.g., once or more daily, weekly, monthly, annually, biannually, etc.). Such samples can be used to verify results from earlier detections and/or to identify an alteration in biological pattern as a result of, for example, disease progression, treatment, remission, and the like. For example, subject samples can be taken and monitored every month, every two months, or combinations of one, two, or three-month intervals according to the present invention. In addition, the biomarker amount and/or activity measurements of the subject obtained over time can be conveniently compared with each other, as well as with those of normal controls during the monitoring period, thereby providing the subject's own values, as an internal, or personal, control for long-term monitoring.

Sample preparation and separation can involve any of the procedures, depending on the type of sample collected and/or analysis of biomarker measurement(s). Such procedures include, by way of example only, concentration, dilution, adjustment of pH, removal of high abundance polypeptides (e.g., albumin, gamma globulin, and transferrin, etc.), addition of preservatives and calibrants, addition of protease inhibitors, addition of denaturants, desalting of samples, concentration of sample proteins, extraction and purification of lipids.

The sample preparation can also isolate molecules that are bound in non-covalent complexes to other protein (e.g., carrier proteins). This process may isolate those molecules bound to a specific carrier protein (e.g., albumin), or use a more general process, such as the release of bound molecules from all carrier proteins via protein denaturation, for example using an acid, followed by removal of the carrier proteins.

Removal of undesired proteins (e.g., high abundance, uninformative, or undetectable proteins) from a sample can be achieved using high affinity reagents, high molecular weight filters, ultracentrifugation and/or electrodialysis. High affinity reagents include antibodies or other reagents (e.g., aptamers) that selectively bind to high abundance proteins. Sample preparation could also include ion exchange chromatography, metal ion affinity chromatography, gel filtration, hydrophobic chromatography, chromatofocusing, adsorption chromatography, isoelectric focusing and related techniques. Molecular weight filters include membranes that separate molecules on the basis of size and molecular weight. Such filters may further employ reverse osmosis, nanofiltration, ultrafiltration and microfiltration.

Ultracentrifugation is a method for removing undesired polypeptides from a sample. Ultracentrifugation is the centrifugation of a sample at about 15,000-60,000 rpm while monitoring with an optical system the sedimentation (or lack thereof) of particles. Electrodialysis is a procedure which uses an electromembrane or semipermeable membrane in a process in which ions are transported through semi-permeable membranes from one solution to another under the influence of a potential gradient. Since the membranes used in electrodialysis may have the ability to selectively transport ions having positive or negative charge, reject ions of the opposite charge, or to allow species to migrate through a semipermeable membrane based on size and charge, it renders electrodialysis useful for concentration, removal, or separation of electrolytes.

Separation and purification in the present invention may include any procedure known in the art, such as capillary electrophoresis (e.g., in capillary or on-chip) or chromatography (e.g., in capillary, column or on a chip). Electrophoresis is a method that can be used to separate ionic molecules under the influence of an electric field. Electrophoresis can be conducted in a gel, capillary, or in a microchannel on a chip. Examples of gels used for electrophoresis include starch, acrylamide, polyethylene oxides, agarose, or combinations thereof. A gel can be modified by its cross-linking, addition of detergents, or denaturants, immobilization of enzymes or antibodies (affinity electrophoresis) or substrates (zymography) and incorporation of a pH gradient. Examples of capillaries used for electrophoresis include capillaries that interface with an electrospray.

Capillary electrophoresis (CE) is preferred for separating complex hydrophilic molecules and highly charged solutes. CE technology can also be implemented on microfluidic chips. Depending on the types of capillary and buffers used, CE can be further segmented into separation techniques such as capillary zone electrophoresis (CZE), capillary isoelectric focusing (CIEF), capillary isotachophoresis (cITP) and capillary electrochromatography (CEC). An embodiment to couple CE techniques to electrospray ionization involves the use of volatile solutions, for example, aqueous mixtures containing a volatile acid and/or base and an organic such as an alcohol or acetonitrile.

Capillary isotachophoresis (cITP) is a technique in which the analytes move through the capillary at a constant speed but are nevertheless separated by their respective mobilities. Capillary zone electrophoresis (CZE), also known as free-solution CE (FSCE), is based on differences in the electrophoretic mobility of the species, determined by the charge on the molecule, and the frictional resistance the molecule encounters during migration, which is often directly proportional to the size of the molecule. Capillary isoelectric focusing (CIEF) allows weakly-ionizable amphoteric molecules, to be separated by electrophoresis in a pH gradient. CEC is a hybrid technique between traditional high performance liquid chromatography (HPLC) and CE.

Separation and purification techniques used in the present invention include any chromatography procedures known in the art. Chromatography can be based on the differential adsorption and elution of certain analytes or partitioning of analytes between mobile and stationary phases. Different examples of chromatography include, but not limited to, liquid chromatography (LC), gas chromatography (GC), high performance liquid chromatography (HPLC), etc.

In some embodiments, whole blood is collected from the subject, and the plasma layer is separated by centrifugation. Cell free DNA may be then extracted from the plasma using methods known in the art. The isolated cell free DNA can be used to detect methylation of genomic loci (i.e., the DMRs listed in Tables 1-8 and/or Tables 12-27) or other genomic and/or epigenomic alterations of biomarkers associated with NEPC.

### IV. Analyzing Genomic and/or Epigenomic Alterations of Biomarker

Genomic and/or epigenomic alterations of a biomarker or panel of biomarkers can be analyzed according to the methods described herein and techniques known to the skilled artisan.

Methods for Detection of Methylated Biomarkers

The detection of hypermethylation or hypomethylation of DNA in a sample can be detected and quantified by any of a number of well-known methods. One method for detecting methylation of DNA in a sample is methylated DNA immunoprecipitation (Me-DIP) that uses methyl DNA specific antibody, or methyl capture using methyl-CpG binding domain (MBD) proteins. Methylation of genomic DNA (gDNA), cell free DNA (cfDNA), or circulating tumor DNA (ctDNA) can be performed using Me-DIP. cfMe-DIP can be performed on samples comprising 5-10 ng, or less, of cfDNA, which can be obtained from about 1 ml of plasma.

Other methods to detect methylation of DNA in a sample include, but are not limited to, differential enzymatic cleavage of DNA, digestion followed by PCR or sequencing, bisulfite conversion followed by methylation-specific PCR or sequencing, whole genome bisulfite sequencing (WGBS), PCR with high resolution melting, COLD-PCR for the detection of unmethylated islands, reduced representation bisulfite sequencing (RRBS), methyl-sensitive cut counting (MSCC), high performance liquid chromatography-ultraviolet (HPLC-UV), liquid chromatography coupled with tandem mass spectrometry (LC-MS/MS), ELISA, amplification fragment length polymorphism (AFLP), restriction fragment length polymorphism (RFLP), bead array (e.g., HumanMethylation450 BeadChip array), luminometric methylation assay (LUMA), LINE-1/pyrosequencing, and affinity capture of methylated DNA (Laird (2010) Nat. Rev. Genet. 11:191-203; Kurdyukov et al. (2016) Biology 5:3). Restriction enzyme based differential cleavage of methylated DNA may be locus-specific. Affinity-capture and bisulphite conversion followed by sequencing methods may be used for both gene specific or genome-wide analysis (Beck (2010) Nat. Biotech. 28:1026-1028).

Methods for detecting epigenetic alterations other than methylation are contemplated herein as are methods for detecting genomic alterations other than epigenetic alterations. Any of the contemplated methods can be used in combination with the method or methods for detecting methylation of genomic loci, such as those listed in Tables 1-8 and/or Tables 12-27. The additional methods include, but are not limited to, methods for detecting mutations or variants, alterations in copy number, and alterations in the expression of biomarker expression.

### V. Anti-Cancer Therapies

NEPC commonly presents as a morphologically mixed tumor in the metastatic CRPC setting when a biopsy is obtained following progression on abiraterone or enzalutamide. The National Comprehensive Cancer Network (NCCN) Prostate Cancer guidelines state that if histologic evidence of both PRAD and NEPC is present, selection of subsequent treatment to target either the PRAD or NEPC component can be prioritized based on the clinical context. Given that NEPC is androgen indifferent and characteristically unresponsive to androgen-deprivation therapy, clinicians tend to favor treating the NEPC component when identified. Clinical features such as significantly discordant PSA and burden of disease or morphologic features of NEPC may suggest benefit to prioritizing therapy targeting NEPC.

The optimal therapeutic approach for patients with NEPC is not clearly defined, but the NCCN Prostate Cancer guidelines refer clinicians to the guidelines for small cell lung cancer (SCLC). Historically, first-line treatment for SCLC was etoposide plus platinum-based chemotherapy, a regimen that would not be used for PRAD. Several clinical studies support the use of platinum-based chemotherapy in NEPC. A retrospective analysis of metastatic castration-resistant prostate cancer (mCRPC) patients treated with platinum chemotherapy found that patients with de novo or treatment-emergent NEPC had a significantly higher response rate (63%) than those with PRAD (14%) (Humeniuk et al. (2018) Prostate Cancer Prostatic Diseases 21(1):92-9). In a Phase 2 study of 13 patients with NEPC (including 11 with pure or mixed NEPC) cisplatin-based chemotherapy resulted in an objective response rate of 66.6% with one complete response (Sella et al. (2000) European Urology 38(3):250-4). Another Phase 2 study of 36 patients with NEPC demonstrated an objective response rate of 61% for the combination of doxorubicin, etoposide, and cisplatin (Papandreou et al. (2002) J. Clin. Oncology 20(14):3072-80).

Recently, however, the IMpower133 and CASPIAN studies independently showed an overall survival benefit for the addition of atezolizumab and durvalumab, respectively, to first-line platinum and etoposide chemotherapy in SCLC (Horn et al. (2018) NEJM 379(23):2220-29; Paz-Arez et al. (2019) Lancet 394(10212):1929-39). These are now the recommended first-line treatments for metastatic SCLC in the NCCN guidelines. Additionally, pembrolizumab is approved for metastatic SCLC following platinum-based chemotherapy and at least one additional line of prior therapy (Chung et al. (2020) J. Thorac. Oncol. 15(4):618-27). These chemoimmunotherapy regimens may now be considered for off-label use in NEPC, but would not be recommended for patients with PRAD.

Thus, methods described herein can be used to assess whether a subject has or is at risk for developing NEPC and based on this diagnosis, the subject may be administered a therapeutically effective amount of an agent. In some embodiments, a subject having NEPC is administered an anti-cancer therapy (e.g., platinum-based chemotherapy). Because NEPC can arise clonally from prostate adenocarcinoma cells in response to treatment with androgen receptor signaling inhibitors and thus have resistance to such treatment, the anti-cancer therapy administered to a subject having or suspected of having NEPC can be a therapy other than an androgen receptor (AR)-targeted therapy. In other embodiments, the subject is determined using the methods herein to not have NEPC or is not at risk of developing NEPC and is administered an AR-targeted therapy.

The anti-cancer therapy is selected from the group consisting of an epigenetic modifier, targeted therapy, chemotherapy, radiation therapy, and/or hormonal therapy, optionally wherein the anti-cancer therapy comprises an AR-targeted therapy.

Combination therapies are also contemplated and can comprise, for example, one or more chemotherapeutic agents and radiation, one or more chemotherapeutic agents and immunotherapy, or one or more chemotherapeutic agents, radiation and chemotherapy, each combination of which can be with the AR-targeted therapy.

The term “targeted therapy” refers to administration of agents that selectively interact with a chosen biomolecule to thereby treat cancer. One example includes immunotherapies such as immune checkpoint inhibitors, which are well known in the art. For example, anti-PD-L1 pathway agents, such as therapeutic monoclonal blocking antibodies, which are well-known in the art and described above, can be used to target tumor microenvironments and cells expressing unwanted components of the PD-1 pathway, such as PD-1, PD-L1, and/or PD-L2.

In some embodiments, a subject having a methylation profile such as those described herein that is indicative of NEPC (e.g., comprising one or more methylated genomic loci listed in Tables 1-8 and/or Tables 12-27) is administered a targeted therapy, such as immunotherapy. In some embodiments, the subject is administered a targeted therapy (e.g., a PD-1 and/or PD-L1 inhibitor) and a chemotherapy. In some embodiments, the subject is administered a targeted therapy and a platinum-based therapy. In some embodiments, the targeted therapy comprises an antibody-based targeted therapy (e.g., an anti-PD-1 antibody and/or an anti-PD-L1 antibody). Representative, non-limiting immunotherapies that target the PD-1 pathway include nivolumab (Opdivo®), atezolizumab (Tecentriq®), and pembrolizumab (Keytruda®). In some embodiments, treatment of NEPC comprises combining a chemotherapy and an immunotherapy.

For example, the term “PD-1 pathway” refers to the PD-1 receptor and its ligands, PD-L1 and PD-L2. “PD-1 pathway inhibitors” block or otherwise reduce the interaction between PD-1 and one or both of its ligands such that the immunoinhibitory signaling otherwise generated by the interaction is blocked or otherwise reduced Immune checkpoint inhibitors can be direct or indirect. Direct immune checkpoint inhibitors block or otherwise reduce the interaction between an immune checkpoint and at least one of its ligands. For example, PD-1 inhibitors can block PD-1 binding with one or both of its ligands. Direct PD-1 combination inhibitors are well-known in the art, especially since the natural binding partners of PD-1 (e.g., PD-L1 and PD-L2), PD-L1 (e.g., PD-1 and B7-1), and PD-L2 (e.g., PD-1 and RGMb) are known.

For example, agents which directly block the interaction between PD-1 and PD-L1, PD-1 and PD-L2, PD-1 and both PD-L1 and PD-L2, such as a bispecific antibody, can prevent inhibitory signaling and upregulate an immune response (i.e., as a PD-1 pathway inhibitor). Alternatively, agents that indirectly block the interaction between PD-1 and one or both of its ligands can prevent inhibitory signaling and upregulate an immune response. For example, B7-1 or a soluble form thereof, by binding to a PD-L1 polypeptide indirectly reduces the effective concentration of PD-L1 polypeptide available to bind to PD-1. Exemplary agents include monospecific or bispecific blocking antibodies against PD-1, PD-L1, and/or PD-L2 that block the interaction between the receptor and ligand(s); a non-activating form of PD-1, PD-L1, and/or PD-L2 (e.g., a dominant negative or soluble polypeptide), small molecules or peptides that block the interaction between PD-1, PD-L1, and/or PD-L2; fusion proteins (e.g. the extracellular portion of PD-1, PD-L1, and/or PD-L2, fused to the Fc portion of an antibody or immunoglobulin) that bind to PD-1, PD-L1, and/or PD-L2 and inhibit the interaction between the receptor and ligand(s); a non-activating form of a natural PD-1, PD-L2, and/or PD-L2 ligand, and a soluble form of a natural PD-1, PD-L2, and/or PD-L2 ligand.

Indirect immune checkpoint inhibitors block or otherwise reduce the immunoinhibitory signaling generated by the interaction between the immune checkpoint and at least one of its ligands. For example, an inhibitor can block the interaction between PD-1 and one or both of its ligands without necessarily directly blocking the interaction between PD-1 and one or both of its ligands. For example, indirect inhibitors include intrabodies that bind the intracellular portion of PD-1 and/or PD-L1 required to signal to block or otherwise reduce the immunoinhibitory signaling. Similarly, nucleic acids that reduce the expression of PD-1, PD-L1, and/or PD-L2 can indirectly inhibit the interaction between PD-1 and one or both of its ligands by removing the availability of components for interaction. Such nucleic acid molecules can block PD-L1, PD-L2, and/or PD-L2 transcription or translation.

Immunotherapies that are designed to elicit or amplify an immune response are referred to as “activation immunotherapies” Immunotherapies that are designed to reduce or suppress an immune response are referred to as “suppression immunotherapies.” Any agent believed to have an immune system effect on the genetically modified transplanted cancer cells can be assayed to determine whether the agent is an immunotherapy and the effect that a given genetic modification has on the modulation of immune response. In some embodiments, the immunotherapy is cancer cell-specific. In some embodiments, immunotherapy can be “untargeted,” which refers to administration of agents that do not selectively interact with immune system cells, yet modulates immune system function. Representative examples of untargeted therapies include, without limitation, chemotherapy, gene therapy, and radiation therapy.

Immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen) Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumor or cancer.

In one embodiment, immunotherapy comprises adoptive cell-based immunotherapies. Well-known adoptive cell-based immunotherapeutic modalities, including, without limitation, irradiated autologous or allogeneic tumor cells, tumor lysates or apoptotic tumor cells, antigen-presenting cell-based immunotherapy, dendritic cell-based immunotherapy, adoptive T cell transfer, adoptive CAR T cell therapy, autologous immune enhancement therapy (AIET), cancer vaccines, and/or antigen presenting cells. Such cell-based immunotherapies can be further modified to express one or more gene products to further modulate immune responses, such as expressing cytokines like GM-CSF, and/or to express tumor-associated antigen (TAA) antigens, such as Mage-1, gp-100, patient-specific neoantigen vaccines, and the like.

In another embodiment, immunotherapy comprises non-cell-based immunotherapies. In one embodiment, compositions comprising antigens with or without vaccine-enhancing adjuvants are used. Such compositions exist in many well-known forms, such as peptide compositions, oncolytic viruses, recombinant antigen comprising fusion proteins, and the like. In still another embodiment, immunomodulatory interleukins, such as IL-2, IL-6, IL-7, IL-12, IL-17, IL-23, and the like, as well as modulators thereof (e.g., blocking antibodies or more potent or longer lasting forms) are used. In yet another embodiment, immunomodulatory cytokines, such as interferons, G-CSF, imiquimod, TNFalpha, and the like, as well as modulators thereof (e.g., blocking antibodies or more potent or longer lasting forms) are used. In another embodiment, immunomodulatory chemokines, such as CCL3, CCL26, and CXCL7, and the like, as well as modulators thereof (e.g., blocking antibodies or more potent or longer lasting forms) are used. In another embodiment, immunomodulatory molecules targeting immunosuppression, such as STATS signaling modulators, NFkappaB signaling modulators, and immune checkpoint modulators, are used. The terms “immune checkpoint” and “immune checkpoint therapy” are described above.

Similarly, agents and therapies other than immunotherapy or in combination thereof can be used with in combination with biomarker inhibitor/immunotherapies to stimulate an immune response to thereby treat a condition that would benefit therefrom. For example, chemotherapy, radiation, epigenetic modifiers (e.g., histone deacetylase (HDAC) modifiers, methylation modifiers, phosphorylation modifiers, and the like), targeted therapy, and the like are well known in the art.

The term “untargeted therapy” refers to administration of agents that do not selectively interact with a chosen biomolecule yet treat cancer. Representative examples of untargeted therapies include, without limitation, chemotherapy, gene therapy, and radiation therapy.

In one embodiment, chemotherapy is used. Chemotherapy includes the administration of a chemotherapeutic agent. Such a chemotherapeutic agent may be, but is not limited to, those selected from among the following groups of compounds: platinum compounds, cytotoxic antibiotics, antimetabolites, anti-mitotic agents, alkylating agents, arsenic compounds, DNA topoisomerase inhibitors, taxanes, nucleoside analogues, plant alkaloids, and toxins, and synthetic derivatives thereof. Exemplary compounds include, but are not limited to, alkylating agents: cisplatin, treosulfan, and trofosfamide; plant alkaloids: etoposide, vinblastine, paclitaxel, docetaxol; DNA topoisomerase inhibitors: teniposide, crisnatol, and mitomycin; anti-folates: methotrexate, mycophenolic acid, and hydroxyurea; pyrimidine analogs: 5-fluorouracil, doxifluridine, and cytosine arabinoside; purine analogs: mercaptopurine and thioguanine; DNA antimetabolites: 2′-deoxy-5-fluorouridine, aphidicolin glycinate, and pyrazoloimidazole; and antimitotic agents: halichondrin, colchicine, and rhizoxin. Compositions comprising one or more chemotherapeutic agents (e.g., FLAG, CHOP) may also be used, and the agents in these compositions can also be used individually. FLAG comprises fludarabine, cytosine arabinoside (Ara-C) and G-CSF. CHOP comprises cyclophosphamide, vincristine, doxorubicin, and prednisone. In another embodiments, PARP (e.g., PARP-1 and/or PARP-2) inhibitors are used and such inhibitors are well-known in the art (e.g., Olaparib, ABT-888, BSI-201, BGP-15 (N-Gene Research Laboratories, Inc.); Rubraca (Clovis Oncology); INO-1001 (Inotek Pharmaceuticals Inc.); PJ34 (Soriano et al., 2001; Pacher et al., 2002b); 3-aminobenzamide (Trevigen); 4-amino-1, 8-naphthalimide; (Trevigen); 6(5H)-phenanthridinone (Trevigen); benzamide (U.S. Pat. Re. 36,397); and NU1025 (Bowman et al.). The mechanism of action is generally related to the ability of PARP inhibitors to bind PARP and decrease its activity. PARP catalyzes the conversion of .beta.-nicotinamide adenine dinucleotide (NAD+) into nicotinamide and poly-ADP-ribose (PAR). Both poly (ADP-ribose) and PARP have been linked to regulation of transcription, cell proliferation, genomic stability, and carcinogenesis (Bouchard V. J. et.al. Experimental Hematology, Volume 31, Number 6, June 2003, pp. 446-454(9); Herceg Z.; Wang Z.-Q. Mutation Research/Fundamental and Molecular Mechanisms of Mutagenesis, Volume 477, Number 1, 2 Jun. 2001, pp. 97-110(14)). Poly(ADP-ribose) polymerase 1 (PARP1) is a key molecule in the repair of DNA single-strand breaks (SSBs) (de Murcia J. et al. (1997) Proc. Natl. Acad. Sci. USA 94:7303-7307; Schreiber V, Dantzer F, Ame J C, de Murcia G (2006) Nat. Rev. Mol. Cell Biol. 7:517-528; Wang Z Q, et al. (1997) Genes Dev. 11:2347-2358). Knockout of SSB repair by inhibition of PARP1 function induces DNA double-strand breaks (DSBs) that can trigger synthetic lethality in cancer cells with defective homology-directed DSB repair (Bryant H E, et al. (2005) Nature 434:913-917; Farmer H, et al. (2005) Nature 434:917-921). The foregoing examples of chemotherapeutic agents are illustrative, and are not intended to be limiting.

In another embodiment, radiation therapy is used. The radiation used in radiation therapy can be ionizing radiation. Radiation therapy can also be gamma rays, X-rays, or proton beams. Examples of radiation therapy include, but are not limited to, external-beam radiation therapy, interstitial implantation of radioisotopes (1-125, palladium, iridium), radioisotopes such as strontium-89, thoracic radiation therapy, intraperitoneal P-32 radiation therapy, and/or total abdominal and pelvic radiation therapy. For a general overview of radiation therapy, see Hellman, Chapter 16: Principles of Cancer Management: Radiation Therapy, 6th edition, 2001, DeVita et al., eds., J. B. Lippencott Company, Philadelphia. The radiation therapy can be administered as external beam radiation or teletherapy wherein the radiation is directed from a remote source. The radiation treatment can also be administered as internal therapy or brachytherapy wherein a radioactive source is placed inside the body close to cancer cells or a tumor mass. Also encompassed is the use of photodynamic therapy comprising the administration of photosensitizers, such as hematoporphyrin and its derivatives, Vertoporfin (BPD-MA), phthalocyanine, photosensitizer Pc4, demethoxy-hypocrellin A; and 2BA-2-DMHA.

In another embodiment, surgical intervention can be used to physically remove cancerous cells and/or tissues.

In still another embodiment, hormone therapy is used. Hormonal therapeutic treatments can comprise, for example, hormonal agonists, hormonal antagonists (e.g., flutamide, bicalutamide, tamoxifen, raloxifene, leuprolide acetate (LUPRON), LH-RH antagonists), inhibitors of hormone biosynthesis and processing and steroids (e.g., dexamethasone, retinoids, deltoids, betamethasone, cortisol, cortisone, prednisone, dehydrotestosterone, glucocorticoids, mineralocorticoids, estrogen, testosterone, progestins), vitamin A derivatives (e.g., all-trans retinoic acid (ATRA)), vitamin D3 analogs, antigestagens (e.g., mifepristone, onapristone), or antiandrogens (e.g., cyproterone acetate).

In yet another embodiment, hyperthermia, a procedure in which body tissue is exposed to high temperatures (up to 106° F.) is used. Heat may help shrink tumors by damaging cells or depriving them of substances they need to live. Hyperthermia therapy can be local, regional, and whole-body hyperthermia, using external and internal heating devices. Hyperthermia is usually used with other forms of therapy (e.g., radiation therapy, chemotherapy, and biological therapy) to try to increase their effectiveness. Local hyperthermia refers to heat that is applied to a very small area, such as a tumor. The area may be heated externally with high-frequency waves aimed at a tumor from a device outside the body. To achieve internal heating, one of several types of sterile probes may be used, including thin, heated wires or hollow tubes filled with warm water; implanted microwave antennae; and radiofrequency electrodes. In regional hyperthermia, an organ or a limb is heated. Magnets and devices that produce high energy are placed over the region to be heated. In another approach, called perfusion, some of the patient's blood is removed, heated, and then pumped (perfused) into the region that is to be heated internally. Whole-body heating is used to treat metastatic cancer that has spread throughout the body. It can be accomplished using warm-water blankets, hot wax, inductive coils (like those in electric blankets), or thermal chambers (similar to large incubators). Hyperthermia does not cause any marked increase in radiation side effects or complications. Heat applied directly to the skin, however, can cause discomfort or even significant local pain in about half the patients treated. It can also cause blisters, which generally heal rapidly.

In still another embodiment, photodynamic therapy (also called PDT, photoradiation therapy, phototherapy, or photochemotherapy) is used for the treatment of some types of cancer. It is based on the discovery that certain chemicals known as photosensitizing agents can kill one-celled organisms when the organisms are exposed to a particular type of light. PDT destroys cancer cells with a fixed-frequency laser light in combination with a photosensitizing agent. In PDT, the photosensitizing agent is injected into the bloodstream and absorbed by cells all over the body. The agent remains in cancer cells for a longer time than it does in normal cells. When the treated cancer cells are exposed to laser light, the photosensitizing agent absorbs the light and produces an active form of oxygen that destroys the treated cancer cells. Light exposure must be timed carefully so that it occurs when most of the photosensitizing agent has left healthy cells but is still present in the cancer cells. The laser light used in PDT can be directed through a fiber-optic (a very thin glass strand). The fiber-optic is placed close to the cancer to deliver the proper amount of light. The fiber-optic can be directed through a bronchoscope into the lungs for the treatment of lung cancer or through an endoscope into the esophagus for the treatment of esophageal cancer. An advantage of PDT is that it causes minimal damage to healthy tissue. However, because the laser light currently in use cannot pass through more than about 3 centimeters of tissue (a little more than one and an eighth inch), PDT is mainly used to treat tumors on or just under the skin or on the lining of internal organs. Photodynamic therapy makes the skin and eyes sensitive to light for 6 weeks or more after treatment. Patients are advised to avoid direct sunlight and bright indoor light for at least 6 weeks. If patients must go outdoors, they need to wear protective clothing, including sunglasses. Other temporary side effects of PDT are related to the treatment of specific areas and can include coughing, trouble swallowing, abdominal pain, and painful breathing or shortness of breath. In December 1995, the U.S. Food and Drug Administration (FDA) approved a photosensitizing agent called porfimer sodium, or Photofrin®, to relieve symptoms of esophageal cancer that is causing an obstruction and for esophageal cancer that cannot be satisfactorily treated with lasers alone. In January 1998, the FDA approved porfimer sodium for the treatment of early non-small cell lung cancer in patients for whom the usual treatments for lung cancer are not appropriate. The National Cancer Institute and other institutions are supporting clinical trials (research studies) to evaluate the use of photodynamic therapy for several types of cancer, including cancers of the bladder, brain, larynx, and oral cavity.

In yet another embodiment, laser therapy is used to harness high-intensity light to destroy cancer cells. This technique is often used to relieve symptoms of cancer such as bleeding or obstruction, especially when the cancer cannot be cured by other treatments. It may also be used to treat cancer by shrinking or destroying tumors. The term “laser” stands for light amplification by stimulated emission of radiation. Ordinary light, such as that from a light bulb, has many wavelengths and spreads in all directions. Laser light, on the other hand, has a specific wavelength and is focused in a narrow beam. This type of high-intensity light contains a lot of energy. Lasers are very powerful and may be used to cut through steel or to shape diamonds. Lasers also can be used for very precise surgical work, such as repairing a damaged retina in the eye or cutting through tissue (in place of a scalpel). Although there are several different kinds of lasers, only three kinds have gained wide use in medicine: Carbon dioxide (CO₂) laser—This type of laser can remove thin layers from the skin's surface without penetrating the deeper layers. This technique is particularly useful in treating tumors that have not spread deep into the skin and certain precancerous conditions. As an alternative to traditional scalpel surgery, the CO₂ laser is also able to cut the skin. The laser is used in this way to remove skin cancers. Neodymium:yttrium-aluminum-garnet (Nd:YAG) laser-light from this laser can penetrate deeper into tissue than light from the other types of lasers, and it can cause blood to clot quickly. It can be carried through optical fibers to less accessible parts of the body. This type of laser is sometimes used to treat throat cancers. Argon laser—This laser can pass through only superficial layers of tissue and is therefore useful in dermatology and in eye surgery. It also is used with light-sensitive dyes to treat tumors in a procedure known as photodynamic therapy (PDT). Lasers have several advantages over standard surgical tools, including: Lasers are more precise than scalpels. Tissue near an incision is protected, since there is little contact with surrounding skin or other tissue. The heat produced by lasers sterilizes the surgery site, thus reducing the risk of infection. Less operating time may be needed because the precision of the laser allows for a smaller incision. Healing time is often shortened; since laser heat-seals blood vessels, there is less bleeding, swelling, or scarring. Laser surgery may be less complicated. For example, with fiber optics, laser light can be directed to parts of the body without making a large incision. More procedures may be done on an outpatient basis. Lasers can be used in two ways to treat cancer: by shrinking or destroying a tumor with heat, or by activating a chemical—known as a photosensitizing agent—that destroys cancer cells. In PDT, a photosensitizing agent is retained in cancer cells and can be stimulated by light to cause a reaction that kills cancer cells. CO₂ and Nd:YAG lasers are used to shrink or destroy tumors. They may be used with endoscopes, tubes that allow physicians to see into certain areas of the body, such as the bladder. The light from some lasers can be transmitted through a flexible endoscope fitted with fiber optics. This allows physicians to see and work in parts of the body that could not otherwise be reached except by surgery and therefore allows very precise aiming of the laser beam. Lasers also may be used with low-power microscopes, giving the doctor a clear view of the site being treated. Used with other instruments, laser systems can produce a cutting area as small as 200 microns in diameter—less than the width of a very fine thread. Lasers are used to treat many types of cancer. Laser surgery is a standard treatment for certain stages of glottis (vocal cord), cervical, skin, lung, vaginal, vulvar, and penile cancers. In addition to its use to destroy the cancer, laser surgery is also used to help relieve symptoms caused by cancer (palliative care). For example, lasers may be used to shrink or destroy a tumor that is blocking a patient's trachea (windpipe), making it easier to breathe. It is also sometimes used for palliation in colorectal and anal cancer. Laser-induced interstitial thermotherapy (LITT) is one of the most recent developments in laser therapy. LITT uses the same idea as a cancer treatment called hyperthermia; that heat may help shrink tumors by damaging cells or depriving them of substances they need to live. In this treatment, lasers are directed to interstitial areas (areas between organs) in the body. The laser light then raises the temperature of the tumor, which damages or destroys cancer cells.

The duration and/or dose of treatment with therapies may vary according to the particular therapeutic agent or combination thereof. An appropriate treatment time for a particular cancer therapeutic agent will be appreciated by the skilled artisan. The present invention contemplates the continued assessment of optimal treatment schedules for each cancer therapeutic agent, where the phenotype of the cancer of the subject as determined by the methods of the present invention is a factor in determining optimal treatment doses and schedules.

Any means for the introduction of a polynucleotide into mammals, human or non-human, or cells thereof may be adapted to the practice of this invention for the delivery of the various constructs of the present invention into the intended recipient. In one embodiment of the present invention, the DNA constructs are delivered to cells by transfection, i.e., by delivery of “naked” DNA or in a complex with a colloidal dispersion system. A colloidal system includes macromolecule complexes, nanocapsules, microspheres, beads, and lipid-based systems including oil-in-water emulsions, micelles, mixed micelles, and liposomes. The preferred colloidal system of this invention is a lipid-complexed or liposome-formulated DNA. In the former approach, prior to formulation of DNA, e.g., with lipid, a plasmid containing a transgene bearing the desired DNA constructs may first be experimentally optimized for expression (e.g., inclusion of an intron in the 5′ untranslated region and elimination of unnecessary sequences (Feigner, et al., (1995) Ann. NY Acad. Sci. 126-139). Formulation of DNA, e.g. with various lipid or liposome materials, may then be effected using known methods and materials and delivered to the recipient mammal. See, e.g., Canonico et al. (1994) Am. J. Respir. Cell. Mol. Biol. 10:24-29; Tsan et al. (1995) Am. J. Physiol. 268 (6 Pt 1):L1052-6.; Alton et al. (1993) Nat. Genet. 5:135-142; and U.S. Pat. No. 5,679,647 by Carson et al.

The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs, which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization.

The surface of the targeted delivery system may be modified in a variety of ways. In the case of a liposomal-targeted delivery system, lipid groups can be incorporated into the lipid bilayer of the liposome in order to maintain the targeting ligand in stable association with the liposomal bilayer. Various linking groups can be used for joining the lipid chains to the targeting ligand. Naked DNA or DNA associated with a delivery vehicle, e.g., liposomes, can be administered to several sites in a subject (see below).

Nucleic acids can be delivered in any desired vector. These include viral and non-viral vectors, such as adenovirus vectors, adeno-associated virus vectors, retrovirus vectors, lentivirus vectors, and plasmid vectors. Viruses from which vectors can be derived include herpes simplex virus (HSV), adeno associated virus (AAV), human immunodeficiency virus (HIV), bovine immunodeficiency virus (BIV), and murine leukemia virus (MLV). Nucleic acids can be administered in any desired format that provides a sufficient delivery level, such as, but not limited to in virus particles, liposomes, nanoparticles, and/or complexed to polymers.

The nucleic acids encoding a protein or nucleic acid of interest may be in a plasmid or viral vector, or other vector as is known in the art. Such vectors are well known and any can be selected for a particular application. In one embodiment of the present invention, the gene delivery vehicle comprises a promoter and a demethylase coding sequence. Preferred promoters are tissue-specific promoters and promoters that are activated by cellular proliferation, such as the thymidine kinase and thymidylate synthase promoters. Other preferred promoters include promoters that are activatable by infection with a virus, such as the α- and β-interferon promoters, and promoters that are activatable by a hormone, such as estrogen. Other promoters that can be used include the Moloney virus LTR, the CMV promoter, and the mouse albumin promoter. A promoter may be constitutive or inducible.

In another embodiment, naked polynucleotide molecules are used as gene delivery vehicles, as described in WO 90/11092 and U.S. Pat. No. 5,580,859. Such gene delivery vehicles can be either growth factor DNA or RNA and, in certain embodiments, are linked to killed adenovirus. Curiel et al. (1992) Hum. Gene. Ther. 3:147-154. Other vehicles which can optionally be used include DNA-ligand (Wu et al.(1989) J. Biol. Chem. 264:16985-16987), lipid-DNA combinations (Felgner et al. (1989) Proc. Natl. Acad. Sci. USA 84:7413 7417), liposomes (Wang et al. (1987) Proc. Natl. Acad. Sci. 84:7851-7855) and microprojectiles (Williams et al. (1991) Proc. Natl. Acad. Sci. 88:2726-2730).

A gene delivery vehicle can optionally comprise viral sequences such as a viral origin of replication or packaging signal. These viral sequences can be selected from viruses such as astrovirus, coronavirus, orthomyxovirus, papovavirus, paramyxovirus, parvovirus, picornavirus, poxvirus, retrovirus, togavirus or adenovirus. In a preferred embodiment, the growth factor gene delivery vehicle is a recombinant retroviral vector. Recombinant retroviruses and various uses thereof have been described in numerous references including, for example, Mann et al. (1983) Cell 33:153, Cane and Mulligan (1984) Proc. Nat'l. Acad. Sci. USA 81:6349, Miller et al. (1990) Human Gene Therapy 1:5-14, U.S. Pat. Nos. 4,405,712, 4,861,719, and 4,980,289, and PCT Application Nos. WO 89/02,468, WO 89/05,349, and WO 90/02,806. Numerous retroviral gene delivery vehicles can be utilized in the present invention, including for example those described in EP 0,415,731; WO 90/07936; WO 94/03622; WO 93/25698; WO 93/25234; U.S. Pat. No. 5,219,740; WO 9311230; WO 9310218; Vile and Hart (1983) Cancer Res. 53:3860-3864; Vile and Hart (1983) Cancer Res. 53:962-967; Ram et al. (1993) Cancer Res. 53:83-88; Takamiya et al. (1992) J. Neurosci. Res. 33:493-503; Baba et al. (1993) J. Neurosurg. 79:729-735, U.S. Pat. No. 4,777,127, GB 2,200,651, EP 0,345,242 and WO91/02805.

### VI. Clinical Efficacy

Clinical efficacy can be measured by any method known in the art. For example, the response to a therapy, such as modulators of methylation of genomic loci (e.g., the loci listed in Tables 1-8 and/or Tables 12-27) or other genomic and/or epigenomic alterations, and/or the expression of biomarkers described herein, relates to any response of the cancer, e.g., a tumor, to the therapy, preferably to a change in tumor mass and/or volume after initiation of neoadjuvant or adjuvant chemotherapy. Tumor response may be assessed in a neoadjuvant or adjuvant situation where the size of a tumor after systemic intervention can be compared to the initial size and dimensions as measured by CT, PET, mammogram, ultrasound or palpation and the cellularity of a tumor can be estimated histologically and compared to the cellularity of a tumor biopsy taken before initiation of treatment. Response may also be assessed by caliper measurement or pathological examination of the tumor after biopsy or surgical resection. Response may be recorded in a quantitative fashion like percentage change in tumor volume or cellularity or using a semi-quantitative scoring system such as residual cancer burden (Symmans et al. (2007) J. Clin. Oncol. 25:4414-4422) or Miller-Payne score (Ogston et al., (2003) Breast (Edinburgh, Scotland) 12:320-327) in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment of tumor response may be performed early after the onset of neoadjuvant or adjuvant therapy, e.g., after a few hours, days, weeks or preferably after a few months. A typical endpoint for response assessment is upon termination of neoadjuvant chemotherapy or upon surgical removal of residual tumor cells and/or the tumor bed.

In some embodiments, clinical efficacy of the therapeutic treatments described herein may be determined by measuring the clinical benefit rate (CBR). The clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR=CR+PR+SD over 6 months. In some embodiments, the CBR for a particular anti-immune checkpoint therapeutic regimen is at least 25%, 30%, 35%, 40%, 45%, 50%, 55%, 60%, 65%, 70%, 75%, 80%, 85%, or more.

Additional criteria for evaluating the response to immunotherapies, such as anti-immune checkpoint therapies, are related to “survival,” which includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g., time of diagnosis or start of treatment) and end point (e.g., death, recurrence or metastasis). In addition, criteria for efficacy of treatment can be expanded to include response to chemotherapy, probability of survival, probability of metastasis within a given time period, and probability of tumor recurrence.

For example, in order to determine appropriate threshold values, a particular anti-cancer therapeutic regimen can be administered to a population of subjects and the outcome can be correlated to biomarker measurements that were determined prior to administration of any immunotherapy, such as anti-immune checkpoint therapy. The outcome measurement may be pathologic response to therapy given in the neoadjuvant setting. Alternatively, outcome measures, such as overall survival and disease-free survival can be monitored over a period of time for subjects following immunotherapies for whom biomarker measurement values are known. In certain embodiments, the same doses of immunotherapy agents, if any, are administered to each subject. In related embodiments, the doses administered are standard doses known in the art for those agents used in immunotherapies. The period of time for which subjects are monitored can vary. For example, subjects may be monitored for at least 2, 4, 6, 8, 10, 12, 14, 16, 18, 20, 25, 30, 35, 40, 45, 50, 55, or 60 months. Biomarker measurement threshold values that correlate to outcome of an immunotherapy can be determined using methods such as those described in the Examples section.

### VII. Further Uses and Methods of the Present Invention

The compositions described herein can be used in a variety of diagnostic, prognostic, and therapeutic applications. In any method described herein, such as a diagnostic method, prognostic method, therapeutic method, or combination thereof, all steps of the method can be performed by a single actor or, alternatively, by more than one actor. For example, diagnosis can be performed directly by the actor providing therapeutic treatment. Alternatively, a person providing a therapeutic agent can request that a diagnostic assay be performed. The diagnostician and/or the therapeutic interventionist can interpret the diagnostic assay results to determine a therapeutic strategy. Similarly, such alternative processes can apply to other assays, such as prognostic assays.

a. Screening Methods One aspect of the present invention relates to screening assays, including non-cell based assays and xenograft animal model assays.

In one embodiment, the present invention relates to assays for screening test agents that modulate the methylation of one or more of the genomic loci listed in Tables 1-8 and/or Tables 12-27. In one embodiment, a method for identifying such an agent entails determining the ability of the agent to modulate the methylation of at least one genomic loci described herein.

In one aspect, a cell-free assay is provided to test the ability to modulate methylation of a target nucleic acid molecule. Cell-based assays are also provided that comprise contacting at least one biomarker described herein, with a test agent, and determining the ability of the test agent to modulate the methylation of a target nucleic acid molecule (e.g., a genomic loci listed in Tables 1-8 and/or Tables 12-27). Test the agent's ability to modulate methylation can be accomplished by measuring directly the methylation of the target nucleic acid molecule or by measuring indirect parameters.

The present invention further pertains to novel agents identified by the above-described screening assays. Accordingly, it is within the scope of this invention to further use an agent identified as described herein, such as in an appropriate animal model. For example, an agent identified as described herein can be used in an animal model to determine the efficacy, toxicity, or side effects of treatment with such an agent. Alternatively, an antibody identified as described herein can be used in an animal model to determine the mechanism of action of such an agent.

b. Predictive Medicine

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for detecting methylation at one or more genomic loci (e.g., DMRs listed in Tables 1-8 and/or Tables 12-27) described herein in the context of a biological sample (e.g., blood, serum, cells, or tissue) to thereby determine whether an individual afflicted with neuroendocrine prostate cancer (NEPC) or at risk for developing NEPC. Such assays can be used for prognostic or predictive purpose alone, or can be coupled with a therapeutic intervention to thereby prophylactically treat an individual prior to the onset or after recurrence of a disorder characterized by or associated with biomarker genomic and/or epigenomic alterations. The skilled artisan will appreciate that any method can use one or more (e.g., combinations) of biomarkers described herein, such as those in the tables, figures, examples, and otherwise described in the specification.

The skilled artisan will also appreciate that, in certain embodiments, the methods of the present invention implement a computer program and computer system. For example, a computer program can be used to perform the algorithms described herein. A computer system can also store and manipulate data generated by the methods of the present invention that comprises a plurality of biomarker signal changes/profiles that can be used by a computer system in implementing the methods of this invention. In certain embodiments, a computer system receives biomarker expression data; (ii) stores the data; and (iii) compares the data in any number of ways described herein (e.g., analysis relative to appropriate controls) to determine the state of informative biomarkers from cancerous or pre-cancerous tissue. In other embodiments, a computer system (i) compares the determined expression biomarker level to a threshold value; and (ii) outputs an indication of whether said biomarker level is significantly modulated (e.g., above or below) the threshold value, or a phenotype based on said indication.

In certain embodiments, such computer systems are also considered part of the present invention. Numerous types of computer systems can be used to implement the analytic methods of this invention according to knowledge possessed by a skilled artisan in the bioinformatics and/or computer arts. Several software components can be loaded into memory during operation of such a computer system. The software components can comprise both software components that are standard in the art and components that are special to the present invention (e.g., dCHIP software described in Lin et al. (2004) Bioinformatics 20, 1233-1240; radial basis machine learning algorithms (RBM) known in the art).

The methods of the present invention can also be programmed or modeled in mathematical software packages that allow symbolic entry of equations and high-level specification of processing, including specific algorithms to be used, thereby freeing a user of the need to procedurally program individual equations and algorithms. Such packages include, e.g., Matlab from Mathworks (Natick, Mass.), Mathematica from Wolfram Research (Champaign, Ill.), S-Plus from MathSoft (Seattle, Wash.), R from R Foundation for Statistical Computing (Vienna, Austria), Python from Python Software Foundation (Wilmington, DE), or Perl from Perl Foundation (Holland, MI). Other programs contemplated herein are disclosed in the Examples.

In certain embodiments, the computer comprises a database for storage of biomarker data. Such stored profiles can be accessed and used to perform comparisons of interest at a later point in time. For example, biomarker expression profiles of a sample derived from the non-cancerous tissue of a subject and/or profiles generated from population-based distributions of informative loci of interest in relevant populations of the same species can be stored and later compared to that of a sample derived from the cancerous tissue of the subject or tissue suspected of being cancerous of the subject.

In addition to the exemplary program structures and computer systems described herein, other, alternative program structures and computer systems will be readily apparent to the skilled artisan. Such alternative systems, which do not depart from the above described computer system and programs structures either in spirit or in scope, are therefore intended to be comprehended within the accompanying claims.

c. Diagnostic Assays

The present invention provides, in part, methods, systems, and code for accurately classifying whether a biological sample (e.g., from a subject) is associated with neuroendocrine prostate cancer (NEPC). In some embodiments, the present invention is useful for classifying a subject has or is at risk for developing (NEPC).

Methods are presented for detecting methylation of genomic loci, such as those listed in Tables 1-8 and/or Tables 12-27, that are useful for determining if a subject has or is at risk of developing NEPC. In certain instances, a neuroendocrine prostate cancer (NEPC) enrichment score is computed based on the presence or absence of methylation at a DMR (e.g., the genomic loci listed in Tables 1-8 and/or Tables 12-27) in a confirmed NEPC tissue (i.e., patient derived xenograft (PDX). In certain instances, the statistical algorithm is a single learning statistical classifier system.

Other suitable statistical algorithms are well known to those of skill in the art. For example, learning statistical classifier systems include a machine learning algorithmic technique capable of adapting to complex data sets (e.g., panel of markers of interest) and making decisions based upon such data sets. In some embodiments, a single learning statistical classifier system such as a classification tree (e.g., random forest) is used. In other embodiments, a combination of 2, 3, 4, 5, 6, 7, 8, 9, 10, or more learning statistical classifier systems are used, preferably in tandem. Examples of learning statistical classifier systems include, but are not limited to, those using inductive learning (e.g., decision/classification trees such as random forests, classification and regression trees (C&RT), boosted trees, etc.), Probably Approximately Correct (PAC) learning, connectionist learning (e.g., neural networks (NN), artificial neural networks (ANN), neuro fuzzy networks (NFN), network structures, perceptrons such as multi-layer perceptrons, multi-layer feed-forward networks, applications of neural networks, Bayesian learning in belief networks, etc.), reinforcement learning (e.g., passive learning in a known environment such as naive learning, adaptive dynamic learning, and temporal difference learning, passive learning in an unknown environment, active learning in an unknown environment, learning action-value functions, applications of reinforcement learning, etc.), and genetic algorithms and evolutionary programming Other learning statistical classifier systems include support vector machines (e.g., Kernel methods), multivariate adaptive regression splines (MARS), Levenberg-Marquardt algorithms, Gauss-Newton algorithms, mixtures of Gaussians, gradient descent algorithms, and learning vector quantization (LVQ). In certain embodiments, the method of the present invention further comprises sending the sample classification results to a clinician, e.g., an oncologist.

In some embodiments, the diagnosis of a subject is followed by administering to the individual a therapeutically effective amount of a defined treatment based upon the diagnosis. For example, in some embodiments, the subject is administered an anti-cancer therapy (e.g., platinum-based chemotherapy) other than an AR-targeted therapy if the subject has or is at risk of developing NEPC. In other embodiments, the subject does not have nor is at risk of developing NEPC and is administered an AR-targeted therapy. The anti-cancer therapy is selected from the group consisting of an epigenetic modifier, targeted therapy, chemotherapy, radiation therapy, and/or hormonal therapy, optionally wherein the anti-cancer therapy comprises an AR-targeted therapy.

In some embodiments, the methods presented herein include obtaining a control biological sample. For example, a control biological sample can be from a subject who does not have a prostate cancer (including NEPC) or from a subject who does have a prostate cancer (e.g., NEPC or PRAD). In some embodiments, a control biological sample is derived from a subject during remission or during treatment.

d. Prognostic Assays

The diagnostic methods described herein can furthermore be utilized to identify subjects having or at risk of developing NEPC. An accurate diagnosis of NEPC can be informative regarding effective and ineffective treatments as subjects having NEPC may be resistant to AR targeting therapies but respond well to other therapies (e.g., platinum-based therapies). Thus, the prognostic assays described herein can be used to determine if a subject can be administered an agent (e.g., an agonist, antagonist, peptidomimetic, polypeptide, peptide, nucleic acid, small molecule, an epigenetic modifier, or other drug candidate) to treat a disease or disorder associated with methylation profiles comprising the differentially methylated regions in Tables 1-8 and/or Tables 12-27.

e. Treatment Methods

Therapeutic compositions described herein, such as agents that modulate genomic and/or epigenomic alterations in cancerous cells or tissues can be used in a variety of in vitro and in vivo applications. In one embodiment, the therapeutic agents can be used to treat NEPC. For example, single or multiple agents that modulate methylation of one or more genomic loci (e.g., the genomic loci listed in Tables 1-8 and/or Tables 12-27) alone or in combination with an additional anti-cancer therapy (e.g., chemotherapy, immunotherapy, or AR-targeted therapy) can be used to treat cancers in subjects identified as having or at the risk of developing NEPC.

### VIII. Administration of Agents

Pharmaceutically acceptable compositions are described herein that comprise a therapeutically effective amount of an agent that modulates methylation of genomic loci (e.g., the genomic loci listed in Tables 1-8 and/or Tables 12-27) or other epigenetic or genomic alterations, biomarker expression and/or activity, or any other agent that is used to treat NEPC, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intrarectally, for example, as a cream or foam; or (5) aerosol, for example, as an aqueous aerosol, liposomal preparation or solid particles containing the compound.

The phrase “therapeutically-effective amount” as used herein means that amount of an agent that modulates methylation of genomic loci (e.g., the genomic loci listed in Tables 1-8 and/or Tables 12-27) or other epigenetic or genomic alterations, biomarker expression and/or activity that is effective for producing some desired therapeutic effect, e.g., cancer treatment, at a reasonable benefit/risk ratio.

The phrase “pharmaceutically acceptable” is employed herein to refer to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the subject. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “pharmaceutically acceptable salts” refers to the relatively non-toxic, inorganic and organic acid addition salts of the agents that modulate methylation of genomic loci (e.g., the genomic loci listed in Tables 1-8 and/or Tables 12-27) or other epigenetic or genomic alterations, biomarker expression and/or activity, or any other agent that is used to treat NEPC. These salts can be prepared in situ during the final isolation and purification of the agents, or by separately reacting a purified agent in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like (See, for example, Berge et al. (1977) J. Pharm. Sci. 66:1-19).

In other cases, the agents useful in the methods of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic, inorganic and organic base addition salts of agents that modulates (e.g., inhibits) biomarker expression and/or activity, or expression and/or activity of the complex. These salts can likewise be prepared in situ during the final isolation and purification of the agents, or by separately reacting the purified agent in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like (see, for example, Berge et al., supra).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations useful in the methods of the present invention include those suitable for oral, nasal, topical (including buccal and sublingual), rectal, vaginal, aerosol and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient, which can be combined with a carrier material to produce a single dosage form, will generally be that amount of the compound that produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an agent that modulates (e.g., inhibits) biomarker expression and/or activity, with the carrier and, optionally, one or more accessory ingredients. In general, the formulations are prepared by uniformly and intimately bringing into association a agent with liquid carriers, or finely divided solid carriers, or both, and then, if necessary, shaping the product.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of an agent as an active ingredient. A compound may also be administered as a bolus, electuary or paste.

In solid dosage forms for oral administration (capsules, tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered peptide or peptidomimetic moistened with an inert liquid diluent.

Tablets, and other solid dosage forms, such as dragees, capsules, pills and granules, may optionally be scored or prepared with coatings and shells, such as enteric coatings and other coatings well known in the pharmaceutical-formulating art. They may also be formulated to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions, which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner Examples of embedding compositions that can be used include polymeric substances and waxes. The active ingredient can also be in micro-encapsulated form, if appropriate, with one or more of the above-described excipients.

Liquid dosage forms for oral administration include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active agent may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal administration may be presented as a suppository, which may be prepared by mixing one or more agents with one or more suitable nonirritating excipients or carriers comprising, for example, cocoa butter, polyethylene glycol, a suppository wax or a salicylate, and which is solid at room temperature, but liquid at body temperature and, therefore, will melt in the rectum and release the active agent.

Dosage forms for the topical or transdermal administration of an agent that modulates methylation of genomic loci (e.g., the genomic loci listed in Tables 1-8 and/or Tables 12-27) or other epigenetic or genomic alterations, biomarker expression and/or activity, or any other agent that is used to treat NEPC include powders, sprays, ointments, pastes, creams, lotions, gels, solutions, patches and inhalants. The active component may be mixed under sterile conditions with a pharmaceutically acceptable carrier, and with any preservatives, buffers, or propellants that may be required.

The ointments, pastes, creams and gels may contain, in addition to an agent, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an agent that modulates methylation of genomic loci (e.g., the genomic loci listed in Tables 1-8 and/or Tables 12-27) or other epigenetic or genomic alterations, biomarker expression and/or activity, or any other agent that is used to treat NEP, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The agent that modulates methylation of genomic loci (e.g., the genomic loci listed in Tables 1-8 and/or Tables 12-27) or other epigenetic or genomic alterations, biomarker expression and/or activity, or any other agent that is used to treat NEPC, can be alternatively administered by aerosol. This is accomplished by preparing an aqueous aerosol, liposomal preparation or solid particles containing the compound. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of the agent together with conventional pharmaceutically acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Transdermal patches have the added advantage of providing controlled delivery of an agent to the body. Such dosage forms can be made by dissolving or dispersing the agent in the proper medium. Absorption enhancers can also be used to increase the flux of the peptidomimetic across the skin. The rate of such flux can be controlled by either providing a rate controlling membrane or dispersing the peptidomimetic in a polymer matrix or gel.

Ophthalmic formulations, eye ointments, powders, solutions and the like, are also contemplated as being within the scope of this invention.

Pharmaceutical compositions of this invention suitable for parenteral administration comprise one or more agents in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers, which may be employed in the pharmaceutical compositions of the present invention, include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of an agent that modulates methylation of genomic loci (e.g., the genomic loci listed in Tables 1-8 and/or Tables 12-27) or other epigenetic or genomic alterations, biomarker expression and/or activity, or any other agent that is used to treat NEPC, in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions, which are compatible with body tissue.

When the agents of the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

Actual dosage levels of the active ingredients in the pharmaceutical compositions of this invention may be determined by the methods of the present invention to obtain an amount of the active ingredient, which is effective to achieve the desired therapeutic response for a particular subject, composition, and mode of administration, without being toxic to the subject.

In some embodiments, nucleic acid molecules can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054 3057). The pharmaceutical preparation of a gene therapy vector can include the gene therapy vector in an acceptable diluent or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery vector.

### IX. Kits

The present invention also encompasses kits for detecting methylation of one or more genomic loci, such as those listed in Tables 1-8 and/or Tables 12-27. A kit of the present invention may also include instructional materials disclosing or describing the use of the kit or an antibody of the disclosed invention in a method of the disclosed invention as provided herein. A kit may also include additional components to facilitate the particular application for which the kit is designed. For example, a kit may additionally contain means of detecting the label (e.g., enzyme substrates for enzymatic labels, filter sets to detect fluorescent labels, appropriate secondary labels such as a sheep anti-mouse-HRP, etc.) and reagents necessary for controls (e.g., control biological samples or standards). A kit may additionally include buffers and other reagents recognized for use in a method of the disclosed invention. Non-limiting examples include agents to reduce non-specific binding, such as a carrier protein or a detergent.

### EXAMPLES

### Example 1: Materials and Methods for Examples 2 and 3

### Sample Selection

cfDNA samples were collected from patients with prostate cancer diagnosed and treated at the Dana-Farber Cancer Institute or Brigham and Women's Hospital between April 2003 and February 2020. NEPC patients had advanced prostate cancer with morphologic or immunohistochemical evidence of neuroendocrine differentiation. PRAD patients had prostate adenocarcinoma with no evidence of neuroendocrine differentiation. All patients provided written informed consent, and the use of samples was approved by the Dana-Farber Cancer Institute IRB, following all relevant ethical regulations. The previously-described LuCaP patient-derived xenografts (PDXs) were derived from resected metastatic prostate cancer with informed consent of patient donors under a protocol approved by the University of Washington Human Subjects Division IRB (Nguyen, H. M. et al. (2017) Prostate 77: 654-671).

### Sample Processing

cfDNA samples were processed by the following method. Peripheral blood was collected in EDTA Vacutainer® tubes (BD), and processed within 3 hours of collection. Plasma was separated by centrifugation at 2,500 g for 10 minutes, transferred to microcentrifuge tubes, and centrifuged at 2,500 g at room temperature for 10 minutes, to remove cellular debris. The supernatant was aliquoted into 1-2 mL aliquots and stored at −80° C. until the time of DNA extraction. cfDNA was isolated from 1 mL of plasma, using the Qiagen Circulating Nucleic Acids Kit (Qiagen), and then incubated with proteinase K for 30 minutes at 60° C. cfDNA was eluted in 50 μl AE buffer and stored at −80° C. DNA concentration was measured using the Qubit dsDNA High Sensitivity Assay Kit (ThermoFisher). DNA from the LuCaP PDXs was extracted using the DNeasy® Blood and Tissue Kit (Qiagen). Genomic DNA was sheared using a Covaris Sonicator E220 and AMPure® XP beads (Beckman Coulter) were used to size select 150-250 bp DNA fragments.

cfMeDIP-Seq Protocol

cfMeDIP-seq was performed using previously published methods (Nuzzo, P. V. et al. (2020) Nat. Med. 26: 1041-1043. cfDNA library preparation was performed using KAPA HyperPrep™ Kit (KAPA Biosystems) according to the manufacturer's protocol. In brief, after end-repair and A-tailing, samples were ligated to 18.1 nM per 1 ng of cfDNA of NEBNext adaptor (NEBNext® Multiplex Oligos for Illumina kit, New England BioLabs) by incubating at 20° C. for 20 minutes and were purified with AMPure® XP beads (Beckman Coulter). Eluted libraries were digested using the USER® enzyme (New England BioLabs), followed by purification with AMPure® XP beads (Beckman Coulter). λ DNA was added to prepared libraries to achieve a total amount of 100 ng DNA. This DNA consists of a mixture of unmethylated and in vitro methylated λ amplicons of different CpG densities, similar in size to adaptor-ligated cfDNA libraries. 0.3 ng of methylated and unmethylated Arabidopsis thaliana DNA was added for quality control (Diagenode). MeDIP was performed using the MagMeDIP kit (Diagenode) following the manufacturer's protocol. DNA was heated to 95° C. for 10 minutes and then incubated in an ice water bath for 10 minutes. Samples were partitioned into two 0.2 ml PCR tubes: 10% input control (7.5 μl) and 90% (75 μl) for immunoprecipitation. Samples were purified using the iPure Kit v2 (Diagenode), and eluted in 50 μl of Buffer C. The success of the immunoprecipitation was confirmed using qPCR to detect recovery of the spiked-in methylated and unmethylated Arabidopsis thaliana DNA (Diagenode) per manufacturer's instructions. Samples that did not pass the quality control threshold of <1% recovery of unmethylated control DNA and >99% recovery of methylated control DNA were excluded.

### Next-Generation Sequencing Library Construction

KAPA HiFi Hotstart ReadyMix (KAPA Biosystems) and NEBNext® Multiplex Oligos for Illumina (New England Biolabs) were added to a final concentration of 0.3 μM and libraries were amplified as follows: activation at 95° C. for 3 minutes, amplification cycles of 98° C. for 20 seconds, 65° C. for 15 seconds, 72° C. for 30 seconds, and a final extension of 72° C. for 1 minute Amplified libraries were purified using AMPure® XP beads (Beckman Coulter). Samples were pooled and sequenced (Novogene Corporation, CA) on Illumina HiSeq® 4000 to generate 150 bp paired end reads. Libraries were multiplexed as twelve samples per lane.

### Quality Control and Processing of Sequencing Reads

After sequencing, the quality and quantity of the raw reads were examined using FastQC version 0.11.5 (www.bioinformatics.babraham.ac.uk/projects/fastqc) and MultiQC version 1.7 (Ewels, P. et al. (2016) Bioinforma. Oxf. Engl. 32: 3047-3048). Raw reads were quality and adapter trimmed using Trim Galore! version 0.6.0 (www.bioinformatics.babraham.ac.uk/projects/trim_galore/) using default settings in paired-end mode. The trimmed reads then were aligned to hg19 using Bowtie2 version 2.3.5.1 in paired-end mode and all other settings default (Langmead, B. et al. (2012) Nat. Methods 9: 357-359). The SAMtools version 1.10 software suite was used to convert SAM alignment files to BAM format, sort and index reads, and remove duplicates (Li, H. et al. (2009) Bioinforma. Oxf. Engl. 25: 2078-2079). The R package RSamtools version 2.2.1 was used to calculate the number of unique mapped reads. Saturation analyses to evaluate reproducibility of each library were carried out using the R Bioconductor package MEDIPS® version 1.38.0 (Lienhard, M. et al. (2014) Bioinforma. Oxf. Engl. 30, 284-286). Based on the quality control results, two samples were removed due to low read count or poor CpG coverage.

### Linear Regression-Based Classification of NEPC Based on Cell Free Methylated DNA

Penalized regression models were built to distinguish NEPC and PRAD patient plasma based on cfDNA methylation using a leave-one-out cross-validation approach. All NEPC and PRAD cfDNA samples, except for one withheld test sample, were used as a training set. The genome was binned into 300 base-pair windows and then tested each window for differential methylation between NEPC and PRAD samples using limma-voom (using R package limma version 3.42.0) on TMM-normalized counts (using R package edgeR version 3.28.0) (Law, C. W. et al. (2014) Genome Biol. 15, R29; Robinson, M. D. et al. (2010) Genome Biol. 11: R25. Only bins with a total count above a fixed threshold were tested for differential methylation, where the threshold was set at 20% of the total number of samples across both groups. The search was restricted to bins within annotated CpG islands and FANTOM5 enhancers and excluded regions of high signal or poor mappability Cavalcante, R. G. Bioinformatics 33, 2381-2383 (2017); Amemiya, H. M. et al. (2019) Sci. Rep. 9: 9354. The most significant 1,000 DMRs between NEPC and PRAD samples were selected. Region filtering and normalization were both carried out on the training set alone (with no input from the test sample).

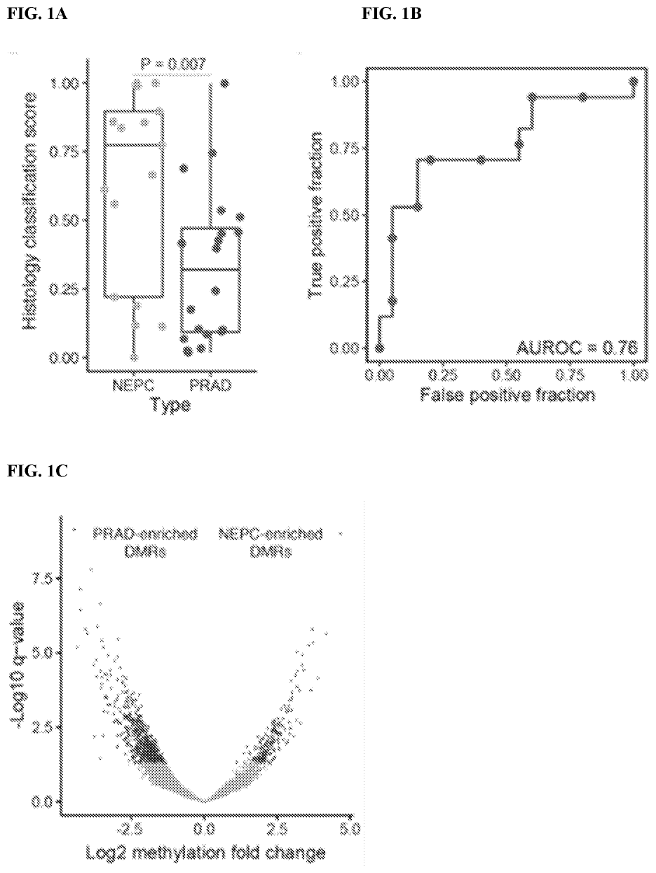

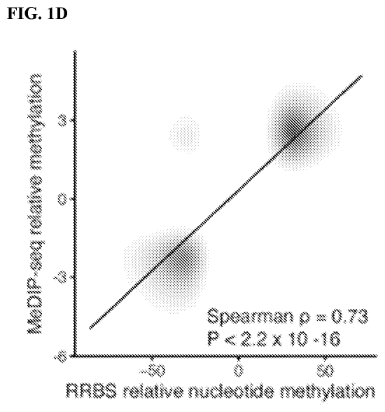

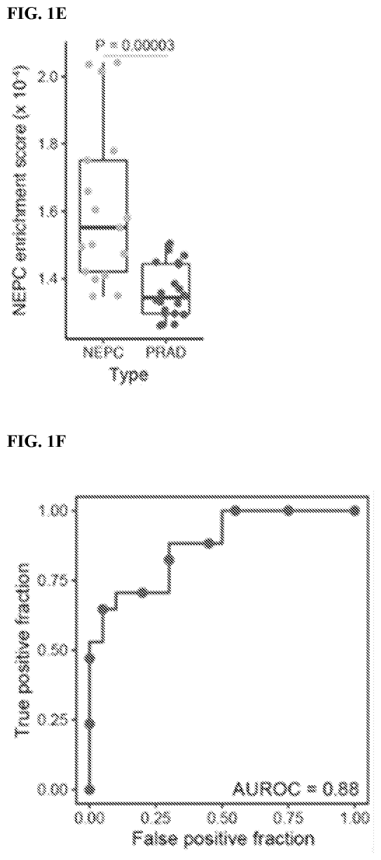

Log-transformed TMM-normalized counts with a pseudo count of 1 in the training set were used to fit a GLMnet model on the 1,000 selected DMRs using the glmnet R package version 3.0-2 (Friedman, J. H. et al. (2010) J. Stat. Softw. 33, 1-22. Three-fold cross-validation was used over a grid of lambda values ranging from 10⁻⁵ to 10² with optimization for AUC. The elastic net mixing parameter alpha was set at 0.2. Each test set was TMM-normalized using the training set as a reference, and log-transformed normalized values with a pseudo count of 1 were fit to the GLMnet model trained on the training set to obtain prediction probabilities of belonging to the NEPC group. These fitted probabilities were used to estimate AUC using the ROCR R package version 1.0-7 (Sing, T. et al. (2005) Bioinformatics 21: 3940-3941. The boxplots in FIG. 1A display the probability for each sample. Differences in the NEPC and PRAD probabilities were calculated using a Wilcoxon rank-sum test.

### Tissue-Informed Methylation Scores

DMRs between NEPC and PRAD were identified in MeDIP-seq data from PDXs, as described above for cfDNA. 6,324 DMRs were selected with read enrichment in NEPC compared to PRAD PDXs at an FDR-adjusted p-value of <0.001 and log₂ fold-change >2. Windows with peaks in MeDIP-seq data from white blood cells (as determined by MACS2, version 2.1.2) were removed to minimize signal from blood cell-derived cfDNA (Zhang, Y. et al. (2008) Genome Biol. 9: R137. Using the MeDIPs R package, CpG-normalized relative methylation scores (rms) were calculated across 300 bp windows for each cfDNA sample (Lienhard, M. et al. (2014) Bioinforma. Oxf. Engl. 30: 284-286. Relative methylation scores were then summed in cfDNA at NEPC-enriched PDX DMRs for each sample, and this value was normalized to the sum of rms values across all 300 bp windows. This normalized methylation score is plotted in FIG. 1E and FIG. 1F.

### Example 2: cfMeDIP-Seq can Distinguish NEPC and PRAD

Given its ability to distinguish cancer types with high accuracy and sensitivity, cfMeDIP-seq was evaluated to determine if this technique could detect the emergence of NEPC early in the disease history based on the distinct methylomes of PRAD and NEPC. Genome-wide methylome maps were generated for 56 samples including plasma cfDNA samples from men with NEPC (N=17) and PRAD (N=20) and genomic DNA from LuCaP patient-derived xenograft (PDX) tumors of NEPC (N=5) and PRAD (N=14) (Nguyen, H. M. et al., (2017) Prostate 77: 654-671. Patients were classified as having NEPC based on morphologic and/or immunohistochemical evidence of neuroendocrine differentiation in tumor tissue. The median age at the time of plasma collection was 69.4 (range 49-86) for NEPC patients and 69.5 (range 54-82) for PRAD patients (Table 10). The median serum PSA level at the time of plasma collection was 1.3 ng/mL (range 0.05-101 ng/mL) for NEPC patients and 63.0 ng/mL (range 6-1457 ng/mL) for PRAD patients.

TABLE 10
Patient characteristics

| | NEPC | PRAD |
|---|---|---|
| N | 17 | 20 |
| Age (median [min, max]) | 69.4 (49, 86) | 69.5 (54, 82) |
| PSA (median [min, max]) | 1.33 (0.05, 101) | 63 (6, 1457) |
| Disease at Plasma Collection | | |
| Localized | 1 | 2 |
| Regional | 1 | 2 |
| Metastatic | 15 | 16 |
| NEPC Type | | |
| De novo | 8 | — |
| Treatment-emergent | 9 | — |

cfMeDIP-seq was first performed on plasma-derived cfDNA from patients with NEPC and PRAD (Shen, S. Y. et al. (2019) Nat. Protoc. 14, 2749-2780. A leave-one-out cross-validation was then performed. Briefly, using all but one cfDNA sample as a training set, the most significant 1,000 differentially methylated regions (DMRs) between the NEPC and PRAD samples were identified. Normalized read counts were used at these DMRs to train a penalized linear regression model to assign a histology classification score to the withheld test sample. Histology classification scores significantly differed between the NEPC and PRAD samples (median 0.77 versus 0.32; P=0.007) (FIG. 1A). The AUROC for accurate classification of NEPC versus PRAD samples using this approach was 0.76 (FIG. 1B).

### Example 3: Tissue-Informed Analysis of DMRs

Since NEPC likely comprises a fraction of circulating tumor DNA, with the majority originating from PRAD, it was assessed whether a supervised analysis based on tumor DNA methylation could improve classification (see Example 1). MeDIP-seq was performed directly in the LuCaP PDXs and DMRs were identified between the NEPC and PRAD samples, resulting in 39,699 NEPC-enriched and 137,692 PRAD-enriched DMRs (FDR adjusted p-value<0.05) (FIG. 1C) (Shen, S. Y. et al. (2019) Nat. Protoc. 14: 2749-2780). Notably, these histology-enriched DMRs were highly concordant (Spearman's correlation coefficient of relative methylation scores=0.73; P<2.2×10⁻¹⁶) with previously published DNA methylation data from an independent dataset of castration-resistant NEPC and PRAD tumors (FIG. 1D) (Beltran, H. et al. (2016) Nat. Med. 22: 298-305). A tissue-informed analysis of the cfDNA samples included assigning an NEPC enrichment score to each sample by summing normalized methylation signals from cfDNA at a subset of the most significant tissue-derived NEPC-enriched DMRs (see Example 1). NEPC enrichment scores were significantly higher for the NEPC samples than the PRAD samples (median 1.55×10⁻⁴ versus 1.34×10⁻⁴; P=0.00003) (FIG. 1E). The AUROC for this tissue-informed approach was 0.88 compared to 0.76 for the tissue-naïve approach (FIG. 1F).

Differentially methylated regions (DMRs) between NEPC (N=5) and PRAD (N=14) patient-derived xenografts were identified from MeDIP-seq data using the R package limma Several thresholds of magnitude and significance were assessed for identifying DMRs, including the log2 fold-difference and FDR-adjusted p-value thresholds listed in Tables 1-8. Windows were removed that had peaks in MeDIP-seq data from white blood cells (as determined by MACS2, version 2.1.2) to minimize signal from blood cell-derived cfDNA.

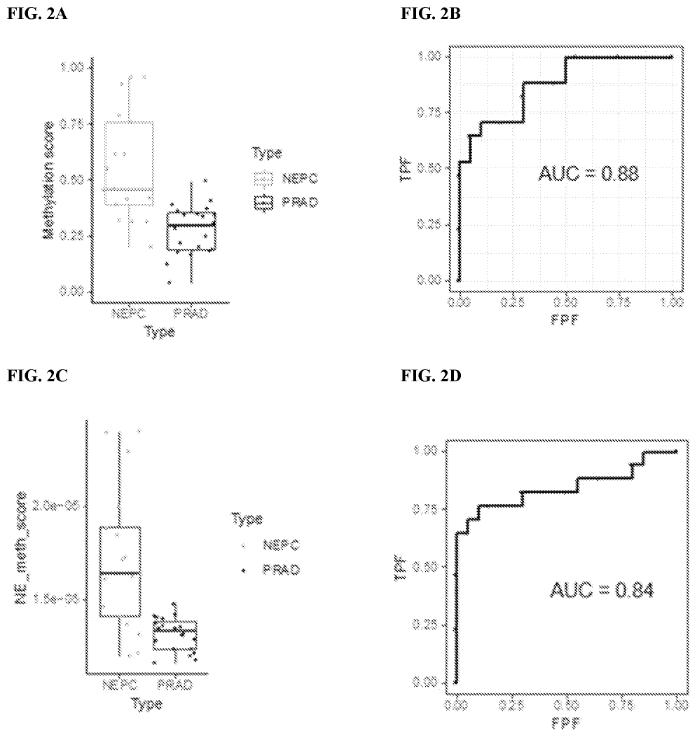

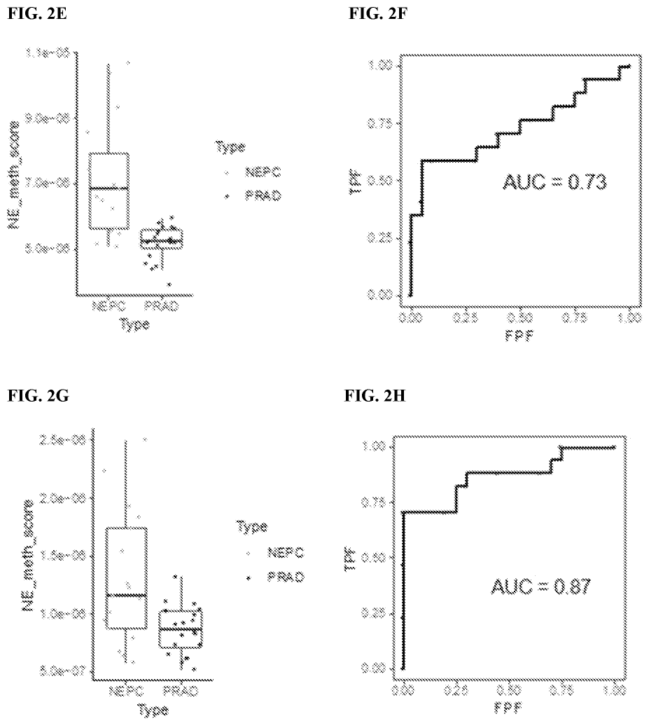

At the DMRs that were more highly methylated in NEPC xenografts (Tables 5-8), cfDNA methylation was assessed in patient plasma samples from cell-free MeDIP-seq data. At each DMR, CpG-normalized relative methylation scores (rms) were calculated in 300 base-pair windows using the MeDIP® R package. The relative methylation scores were summed for cfDNA at NEPC-enriched PDX DMRs for each sample, and this value was normalized to the sum of rms values across all 300 bp windows (FIGS. 2A, 2C, 2E, and 2G). The difference in scores between NEPC and PRAD patient plasma was assessed using the Wilcoxon rank-sum test. Receiver operating characteristic curves (ROC) were generated comparing the true positive and false positive fractions at all possible rms score cutoffs for classifying NEPC (FIGS. 2B, 2D, 2F, and 2H). Thus, of the 1,674 DMRs in Table 5, 1,112 DMRs (Table 1) were identified in this subsequent analysis (FIG. 2A and FIG. 2B). Of the 193 DMRs in Table 6, 124 DMRs (Table 2) were identified in this subsequent analysis (FIG. 2C and FIG. 2D). Of the 76 DMRs in Table 7, 51 DMRs (Table 3) were identified in this subsequent analysis (FIG. 2E and FIG. 2F). Of the 20 DMRs in Table 8, 17 DMRs (Table 4) were identified in this subsequent analysis (FIG. 2G and FIG. 2H).

Successful cfDNA-based biomarkers must be accurate, cost-effective, and practical to implement in routine clinical practice. Beltran et al previously demonstrated the feasibility of detecting NEPC-specific DNA methylation in cfDNA using whole-genome bisulfite sequencing (WGBS) (Beltran, H. et al. (2020) J. Clin. Invest. 130: 1653-1668). Compared to WGBS or targeted bisulfite sequencing, cfMeDIP-seq has several advantages when considering a clinical cfDNA-based biomarker. The high cost of whole-genome sequencing currently limits the ability to implement WGBS in routine clinical practice. In contrast, by sequencing only methylated cfDNA, generally less than 2% of the genome, cfMeDIP-seq provides comprehensive genome-wide methylation data at a fraction of the cost of WGBS (Fouse, S. D. et al. (2010) Epigenomics 2: 105-117). Further, bisulfite sequencing risks losing the majority of cfDNA due to degradation during bisulfite conversion, whereas more than 99% of methylated fragments are retained with cfMeDIP-seq.2,15 This difference can improve tumor detection given the median tumor variant allele frequency in cfDNA is less than 2% even in advanced prostate cancer (Zill, O. A. et al. (2018) Clin. Cancer Res. 24, 3528-3538). This can also improve the ability to detect NEPC in cases of mixed histology or heterogeneous tumors. Finally, cfMeDIP-seq requires only 5-10 ng of cfDNA, which can be obtained from approximately 1 ml of patient plasma.

A non-invasive clinical biomarker that detects NEPC in men with metastatic prostate cancer could have important prognostic and predictive implications. Biopsy-proven NEPC is associated with significantly shorter survival compared to patients with pure adenocarcinoma (Aggarwal, R. et al. (2018) J. Clin. Oncol. 36: 2492-2503). From a therapeutic standpoint, patients with NEPC are characteristically unresponsive to androgen deprivation therapy and potent ARSIs; however, they are more likely to respond to platinum-based chemotherapy than patients with PRAD (Humeniuk, M. S. et al. (2018) Prostate Cancer Prostatic Dis. 21: 92-99). Preliminary data suggest that the presence of NEPC-associated methylation changes in cfDNA is associated with shorter response to ARSIs (Peter, M. R. et al. (2020) Epigenomics 12: 1317-1332).

In summary, the results disclosed herein demonstrate the feasibility of using cfMeDIP-seq to detect NEPC in men with metastatic prostate cancer. This is the first application of cfMeDIP-seq to detect a clinically actionable resistance phenotype. Moreover, a novel tissue-informed approach is described that significantly improves classification performance. NEPC is currently diagnosed by invasive tumor biopsy and is often delayed or missed as only select patients suspected to have this aggressive variant are assessed. The results presented herein indicate that cfMeDIP-seq can be used to non-invasively screen men with metastatic prostate cancer to identify those likely to have NEPC who may benefit from platinum-based chemotherapy or participation in a clinical trial of NEPC-specific therapy.

### Example 4: Materials and Methods for Examples 5-9

### Subjects and Samples

Plasma samples were collected from men with mCRPC diagnosed and treated at the Dana-Farber Cancer Institute (DFCI), Brigham and Women's Hospital (BWH), or Weill Cornell Medicine (WCM) between April 2003 and August 2021. Two genitourinary pathologists confirmed the presence of high-grade neuroendocrine carcinoma of prostate origin according to modern conventions based on histologic review of available material, re-interpretation of original reports, and integration of available molecular results (Epstein, J. I., et al. (2014) Am J Surg Pathol. 2014; 38:756-67). PRAD patients had castration-resistant prostate adenocarcinoma with no pathologic evidence of neuroendocrine differentiation throughout their disease course. All patients provided written informed consent. The use of samples was approved by the DFCI (01-045 and 09-171) and WCM (1305013903). Studies were conducted in accordance with recognized ethical guidelines. The previously-described LuCaP PDXs were derived from resected metastatic prostate cancer with informed consent of patient donors under a protocol approved by the University of Washington Human Subjects Division IRB.

### Sample Processing

cfDNA samples were processed by the following method. Peripheral blood was collected in EDTA Vacutainer tubes (BD), and processed within 3 hours of collection. Plasma was separated by centrifugation at 2,500 g for 10 minutes, transferred to microcentrifuge tubes, and centrifuged at 2,500 g at room temperature for 10 minutes, to remove cellular debris. The supernatant was aliquoted into 1-2 mL aliquots and stored at −80° C. until the time of DNA extraction. cfDNA was isolated from 1 mL of plasma, using the Qiagen Circulating Nucleic Acids Kit (Qiagen), eluted in AE buffer, and stored at −80° C. DNA from the LuCaP PDXs was extracted using the DNeasy® Blood and Tissue Kit (Qiagen). Genomic DNA was sheared using a Covaris Sonicator E220 and AMPure XP beads (Beckman Coulter) were used to size select 150-250 bp DNA fragments.

cfDNA Tumor Content Calculation

Low-pass whole genome sequencing (LPWGS) was performed on all cfDNA samples. The ichorCNA R package was used to infer copy number profiles and cfDNA tumor content from read abundance across bins spanning the genome, using default parameters Adalsteinsson V. A., et al. (2017) Nat Commun.; 8:1324).

cfMeDIP-Seq Protocol

cfMeDIP-seq was performed using previously published methods (Nuzzo, P. V., et al. (2020) Nat Med.26:1041-3). cfDNA library preparation was performed using KAPA HyperPrep Kit (KAPA Biosystems) according to the manufacturer's protocol. End-repair, A-tailing, and ligation of NEBNext adaptors was then prefromed (NEBNext® Multiplex Oligos for Illumina® kit, New England BioLabs). Libraries were digested using the USER enzyme (New England BioLabs). λ DNA, consisting of unmethylated and in vitro methylated DNA, was added to prepared libraries to achieve a total amount of 100 ng DNA. Methylated and unmethylated Arabidopsis thaliana DNA (Diagenode) was added for quality control. MeDIP was performed using the MagMeDIP kit (Diagenode) following the manufacturer's protocol. Samples were purified using the iPure Kit v2 (Diagenode). Success of the immunoprecipitation was confirmed using qPCR to detect recovery of the spiked-in Arabidopsis thaliana methylated and unmethylated DNA.

### Next-Generation Sequencing Library Construction

KAPA HiFi Hotstart ReadyMix (KAPA Biosystems) and NEBNext® Multiplex Oligos for Illumina® (New England Biolabs) were added to a final concentration of 0.3 μM and libraries were amplified as follows: activation at 95° C. for 3 minutes, amplification cycles of 98° C. for 20 seconds, 65° C. for 15 seconds, 72° C. for 30 seconds, and a final extension of 72° C. for 1 minute. Samples were pooled and sequenced (Novogene Corporation, CA) on Illumina® HiSeq 4000 to generate 150 bp paired-end reads.

### Quality Control and Processing of Sequencing Reads

After sequencing, the quality and quantity of the raw reads were examined using FastQC version 0.11.5 (World Wide Web bioinformatics.babraham.ac.uk/projects/fastqc) and MultiQC version 1.7. Raw reads were quality and adapter trimmed using Trim Galore! version 0.6.0 (which can be found on the World Wide Web at bioinformatics.babraham.ac.uk/projects/trim_galore/) using default settings in paired-end mode. The trimmed reads then were aligned to hg19 using Bowtie2 version 2.3.5.1 in paired-end mode and all other settings default.(Langmead, B and Salzberg, S. L. (2012) Nat Methods 9:357-9) The SAMtools version 1.10 software suite was used to convert SAM alignment files to BAM format, sort and index reads, and remove duplicates.(Li H, et al. (2009) Bioinforma Oxf Engl. 2009; 25:2078-9). The R package RS amtools version 2.2.1 was used to calculate the number of unique mapped reads. Saturation analyses to evaluate reproducibility of each library were carried out using the R Bioconductor package MEDIPS version 1.38 (Lienhard, M., (2014) Bioinforma Oxf Engl. 30:284-6).

### Tissue-Informed Approach to NEPC Detection

DMRs were first identified between NEPC and PRAD tumors by binning the genome into 300 base-pair windows and testing each window for differential methylation between NEPC and PRAD samples using limma-voom (using R package limma version 3.42.0) on TMM-normalized counts (using R package edgeR version 3.28.0).(Law C. W., (2014) Genome Biol.15:R29; Robinson, M. D. (2010) Genome Biol.11:R25.) Only bins with a total count above a fixed threshold were tested for differential methylation, where the threshold was set at 20% of the total number of samples across both groups. The search was restricted to bins within annotated CpG islands and FANTOM5 enhancers and excluded regions of high signal or poor mappability.(Cavalcante R. G., (2019) Bioinformatics. Oxford Academic; 33:2381-3; Amemiya H. M., (2019) Sci Rep. 9:9354) DMRs were then selected with read enrichment in NEPC compared to PRAD PDXs at FDR-adjusted P<1.0×10⁻⁶ and log₂ fold-change >3. Windows with peaks were removed in MeDIP-seq data from white blood cells (as determined by MACS2, version 2.1.2) to minimize signal from blood cell-derived cfDNA (Zhang Y, et al. (2008) Genome Biol.; 9:R137). Using the MeDIPs R package, CpG-normalized relative methylation scores were calculated across 300 bp windows for each cfDNA sample (Lienhard M. (2014) Bioinforma Oxf Engl.; 30:284-6; Pelizzola M, et al. (2008) Genome Res.; 18:1652-9). The relative methylation scores were summed in cfDNA at NEPC-enriched PDX DMRs for each sample and normalized this value to the sum of rms values across all 300 bp windows. This value was termed “NEPC Methylation Value.” The same process was performed for PRAD-enriched PDX DMRs to derive a “PRAD Methylation Value.” The log₂ ratio of the NEPC Methylation Value to the PRAD Methylation Value was calculated and these values were normalized to the median score in cfDNA from eight healthy cancer-free controls. This value was termed the “NEPC Risk Score.” This approach is summarized in FIG. 3A.

All code used to process the data and carry out the analyses described in the methods is in a publicly available GitHub repository at the World Wide Web at github.com/scbaca/cfmedip.

### Statistical Analysis

Comparisons between two groups were calculated using a Wilcoxon rank-sum test. To determine the accuracy of the NEPC Risk Score for discriminating between cfDNA samples from men with NEPC versus CR-PRAD, the AUROC was calculated using JMP version 16. The optimal cutoff for classifying NEPC versus CR-PRAD samples based on NEPC Risk Scores in the cfDNA test cohort was calculated using Youden's index (J=sensitivity+specificity−1). The optimal cutoff was determined as the point with the maximum index value. OS was defined as time from radiographic evidence of metastatic disease to death. Living patients were censored at the last evaluation. OS was estimated using the Kaplan-Meier method. P-values were calculated using log-rank test. All P-values were two-sided.

### Example 5: Identification of NEPC- and PRAD-Enriched DMRs in a Tumor Training Set

### Calculation and Generation of NEPC Risk Value Cut-Offs

Neuroendocrine prostate cancer (NEPC) can arise as a resistance mechanism to androgen deprivation therapy (ADT) and androgen receptor signaling inhibitors (ARSIs) in men with metastatic castration-resistant prostate cancer (mCRPC). Present in up to 17% of men with mCRPC, NEPC is associated with poor response to ARSIs and shorter overall survival (OS) (Aggarwal R, et al. (2018) J Clin Oncol Off J Am Soc Clin Oncol. 36:2492-503; Abida W, et al. (2019) Proc Natl Acad Sci US A. 116:11428-36; Abida W, et al. (2019) Proc Natl Acad Sci. National Academy of Sciences; 116:11428-36.) However, NEPC tumors are more likely to respond to platinum-based chemotherapy and several novel NEPC-directed therapies are in clinical development (Humeniuk M. S., et al. (2018) Prostate Cancer Prostatic Dis. 21:92-9).

The current approach to diagnosing NEPC— performing tissue biopsy for pathologic tumor analysis—has significant shortcomings. There is a lack of consensus pathological criteria for defining NEPC and, due to intra-patient tumor heterogeneity, biopsy samples may not represent a patient's overall disease burden (Beltran H, et al. (2016) Nat Med. 22:298-305; Gundem G., et al. (2015) Nature.520:353-7; Beltran H, et al. (2020) J Clin Invest. 130:1653-68). Consequently, NEPC diagnosis is often delayed or missed and reported rates likely underestimate the prevalence of this aggressive disease variant. The lack of a biomarker for early and accurate detection is a significant barrier to improving outcomes for men who develop NEPC.

Liquid biopsies are well-suited to address this unmet need. Most clinical cell-free DNA (cfDNA) tests detect somatically acquired tumor mutations or copy number alterations. However, the defining genetic hallmark of NEPC, deleterious alterations in RB1 and/or TP53, are present in more than one-third of castration-resistant prostate adenocarcinoma (CR-PRAD) tumors and thus cannot unambiguously discriminate between the tumor subtypes (Aggarwal R. et al. (2018) J Clin Oncol Off J Am Soc Clin Oncol. 36:2492-503). In contrast, vast methylation differences exist between NEPC and CR-PRAD. Cell-free methylated DNA immunoprecipitation and high-throughput sequencing (cfMeDIP-seq), a highly sensitive method for genome-wide cfDNA methylation profiling, capable of non-invasive cancer detection and discriminating between tumor types, is well-suited to non-invasively detect NEPC.(Shen S. Y., et al. (2018) Nature; 563:579-83; Shen S. Y. et al. (2019) Nat Protoc.14:2749-80; Nuzzo P V, et al. (2020) Nat Med.; 26:1041-3.)

Herein, the ability of cfMeDIP-seq to detect NEPC in men with mCRPC was evaluated. methylation profiling was first performed on a training set of NEPC and PRAD tumors to identify methylation sites enriched in each tumor type. The ability to implement tissue-informed analysis of cfMeDIP-seq data to detect NEPC in cfDNA from men with NEPC or CR-PRAD was then established. Finally, in an independent cfDNA cohort from men with NEPC or CR-PRAD, the analytical and clinical validity of this approach is confirmed for accurate, non-invasive detection of NEPC.

Prior applications of cfMeDIP-seq for non-invasive cancer detection identified DMRs directly in cfDNA between highly disparate patient groups, such as cancer versus no cancer.

Shen S. Y., et al. (2018) Nature. 563:579-83; Shen S. Y. (2019) Nat Protoc.; 14:2749-80; Nuzzo P. V., et al. (2020) Nat Med.; 26:1041-3.) However, as men with mCRPC who develop NEPC often have concurrent PRAD, this limits the ability to identify NEPC-specific DMRs directly in cfDNA. To address this unique challenge, a novel tissue-informed strategy was developed for analyzing cfMeDIP-seq data (FIG. 3A). MeDIP-seq was performed on 29 LuCaP PDXs, including 5 NEPC and 24 PRAD tumors (Table 11). PDXs were analyzed based on recent single-cell analyses of mCRPC clinical biopsy specimens, which revealed significant intra-tumoral heterogeneity, including admixed NEPC and PRAD cell populations (Cejas P et al. (2021) Nat Commun.; 12:5775; Dong B, et al. (2020) Commun Biol.; 3:778.)

In contrast, the LuCaP PDXs, which have undergone comprehensive pathologic and molecular characterization, provide a more pure source of NEPC and PRAD tumor cells (Nguyen H. M. et al. (2017) The Prostate. 77:654-71

TABLE 11
LuCaP patient-derived xenografts (PDXs)
used for tumor methylation analysis

| LuCaP PDX | Tumor Type |
|---|---|
| 49 | NEPC |
| 93 | NEPC |
| 145.1 | NEPC |
| 145.2 | NEPC |
| 173.1 | NEPC |
| 23.1 | PRAD |
| 23.1CR | PRAD |
| 35 | PRAD |
| 35CR | PRAD |
| 58 | PRAD |
| 70 | PRAD |
| 70CR | PRAD |
| 73 | PRAD |
| 73CR | PRAD |
| 77 | PRAD |
| 77CR | PRAD |
| 78 | PRAD |
| 78CR | PRAD |
| 81 | PRAD |
| 86CR | PRAD |
| 86.2 | PRAD |
| 86.2CR | PRAD |
| 92 | PRAD |
| 96 | PRAD |
| 105 | PRAD |
| 136 | PRAD |
| 136CR | PRAD |
| 147 | PRAD |
| 147CR | PRAD |

Differential methylation analysis of the LuCaP PDXs identified 39,699 NEPC-enriched and 137,692 PRAD-enriched DMRs (FDR-adjusted P<0.05) (FIG. 3B)(Id.) To ensure that the PDX methylation data is representative of clinical biopsy specimens, the LuCaP-derived NEPC- and PRAD-enriched DMRs was compared to DNA methylation data generated from an independent set of castration-resistant NEPC and PRAD tumors using reduced-representation bisulfite sequencing. A high correlation was observed between NEPC- and PRAD-enriched DMRs from the LuCaP PDXs and the clinical biopsy specimens (p=0.73; P<2.2×10⁻¹⁶) (FIG. 3C).

A subset of NEPC- and PRAD-enriched DMRs was identified that could be used to non-invasively detect NEPC. Using a stringent cutoff of FDR-adjusted P<1.0×10⁻⁶ and log₂ fold-change >3, 432 NEPC-enriched and 1,086 PRAD-enriched DMRs were then identified. As the majority of cfDNA is derived from leukocytes, sites that were methylated in WBCs from age-matched male controls (N=1,165), resulting in a final set of 76 NEPC-enriched and 277 PRAD-enriched DMRs. The SPDEF gene highlights the importance of this step. While SPDEF was methylated in NEPC and unmethylated in PRAD tumors (FIG. 3D), it is also methylated in WBCs. The inability to determine whether a methylated cfDNA fragment at this locus originated from NEPC or WBCs renders it uninformative for detecting NEPC and could contribute to misclassification. As exemplified in UNC13A, a gene associated with neural signaling, the final set of DMRs are methylated in one tumor type and unmethylated in the opposite tumor type and WBCs. Consequently, cfDNA fragments at these loci indicate the presence of NEPC or PRAD.

To ensure that the final set of tumor-derived DMRs retained biological relevance, nearby genes for Gene Ontology (GO) term enrichment were assessed.(McLean CY, et al. (2020) Nat Biotechnol. 28:495-501) The top GO terms in NEPC-enriched DMRs pertained to neural development and differentiation, whereas PRAD-enriched DMRs related to hormone signaling and epithelial cell differentiation, suggesting that the final set of tumor-derived DMRs reflect divergent gene regulatory programs of NEPC and PRAD (FIG. 3E).

### Example 6: Classification of NEPC and CR-PRAD Samples in a cfDNA Test Cohort (Calculation and Generation of NEPC Risk Value Cut-Offs)

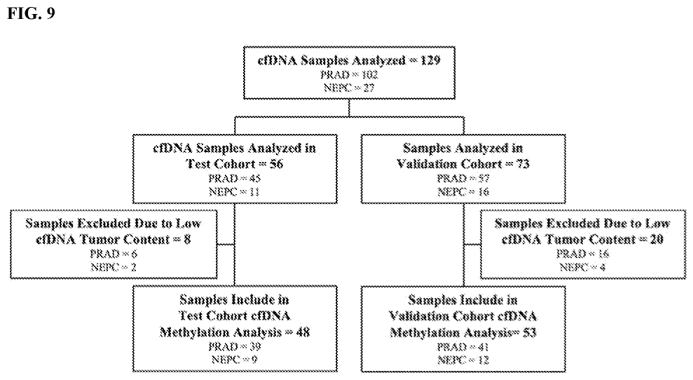

To evaluate the ability to accurately detect NEPC using the novel tissue-informed approach, a test cohort of plasma cfDNA samples from 56 men with mCRPC was analyzed, including 11 with NEPC and 45 with CR-PRAD. LPWGS was first performed on all samples and utilized ichorCNA to estimate cfDNA tumor content. Based on the ichorCNA lower limit of detection (3%), 48 (86%) of the 57 cfDNA samples had detectable tumor DNA including 9 (82%) and 39 (87%) of NEPC and CR-PRAD patients, respectively (Adalsteinsson, V. A., et al. (2017) Nat Commun. 8:1324) Samples with cfDNA tumor content less than 3% were excluded from the cfDNA methylation analysis FIG. 9). These results compare favorably to a published cfDNA analysis of 269 samples from men with metastatic prostate cancer that detected tumor DNA in 83% of samples using LPWGS and hybrid-capture targeted sequencing Mayrhofer, M., et al. (2018) Genome Med.10:85).

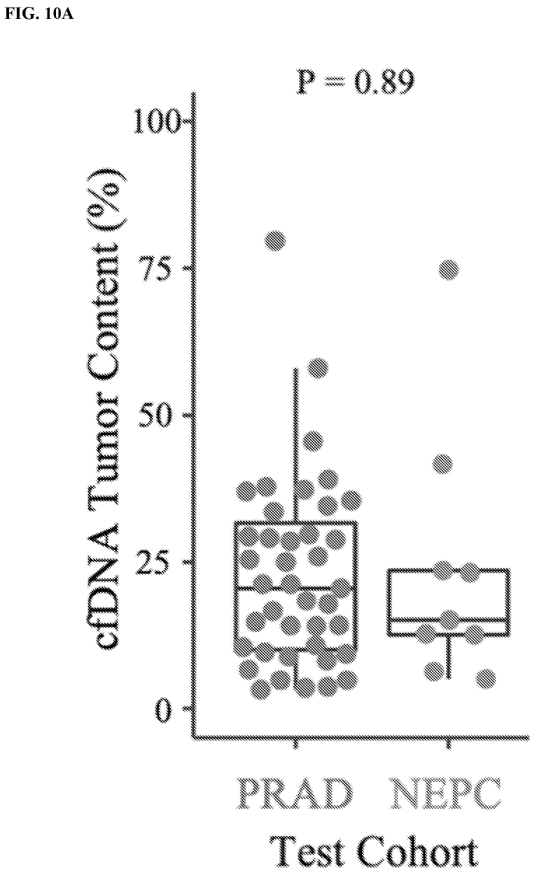

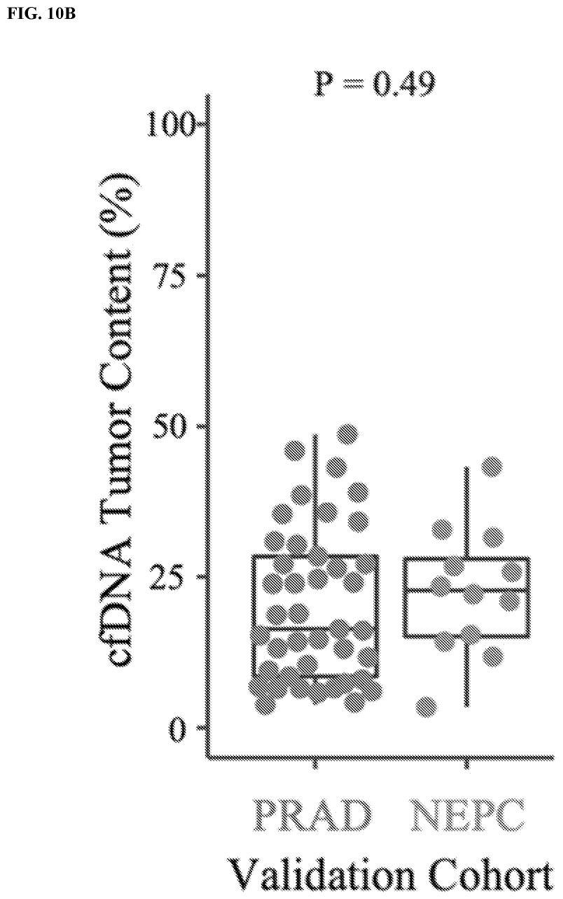

Characteristics of men in the cfDNA test cohort at the time of plasma collection are listed in Table 9. Consistent with known decoupling of prostate specific antigen (PSA) from its typical association with disease burden in NEPC, the median PSA was 0.37 for NEPC patients (range 0.03-3.7) and 140 for CR-PRAD patients (range 0.79-4305).(11) Median cfDNA tumor content was 15% for men with NEPC (range 5.1-75%) and 21% for those with CR-PRAD (range 3.3-80%) (P=0.89) (Table 9; FIG. 10).

TABLE 9
Patient characteristics at the time of cfDNA collection
in the test and validation cohorts of men with mCRPC.

| | Test Cohort | | Validation Cohort | |
|---|---|---|---|---|
| | NEPC | PRAD | NEPC | PRAD |
| | N = 9 | N = 39 | N = 12 | N = 41 |
| Median cfDNA | 15% | 21% | 23% | 16% |
| Tumor Content | (5.1-75%) | (3.3-80%) | (3.4-43%) | (3.8-49%) |
| (Range) | | | | |
| Median Age | 72 | 71 | 71 | 70 |
| (Range) | (60-84) | (61-92) | (54-91) | (49-86) |
| Median PSA | 0.37 | 140 | 0.33 | 112 |
| (Range) | (0.03-3.7) | (0.79-4305) | (0.01-6.23) | (4.5-1821) |
| De Novo NEPC | 3 (33%) | N/A | 2 (17%) | N/A |
| Prior Local | 5 (56%) | 27 (69%) | 5 (42%) | 26 (63%) |
| Therapy | | | | |
| Prior ADT | 4 (44%) | 39 (100%) | 8 (67%) | 41 (100%) |
| Prior | 0 (0%) | 36 (92%) | 4 (33%) | 39 (95%) |
| Abiraterone or | | | | |
| Enzalutamide | | | | |
| Prior Docetaxel | 2 (22%) | 25 (64%) | 2 (17%) | 35 (85%) |
| Prior EP | 7 (78%) | 0 (0%) | 8 (67%) | 0 (0%) |
| Chemotherapy | | | | |
| Liver Metastases | 3 (33%) | 15 (38%) | 8 (67%) | 13 (32%) |

Abbreviations:
mCRPC, metastatic castration-resistant prostate cancer;
cfDNA, cell-free DNA;
NEPC, neuroendocrine prostate cancer;
PRAD, prostate adenocarcinoma;
PSA, prostate-specific antigen;
N/A, not applicable;
ADT, androgen deprivation therapy;
ARSI, androgen receptor signaling inhibitor;
EP, etoposide plus platinum

To evaluate to the ability to detect NEPC in cfDNA from men with mCRPC, cfMeDIP-seq was first performed on the test cohort samples. An NEPC Methylation Value and PRAD Methylation Value was calculated for each sample by summing the methylated cfDNA fragments at tissue-derived NEPC-enriched and PRAD-enriched DMRs, respectively (FIG. 3A). An NEPC Risk Score was calculated for each sample as the normalized ratio of the NEPC Methylation Value versus the PRAD Methylation Value.

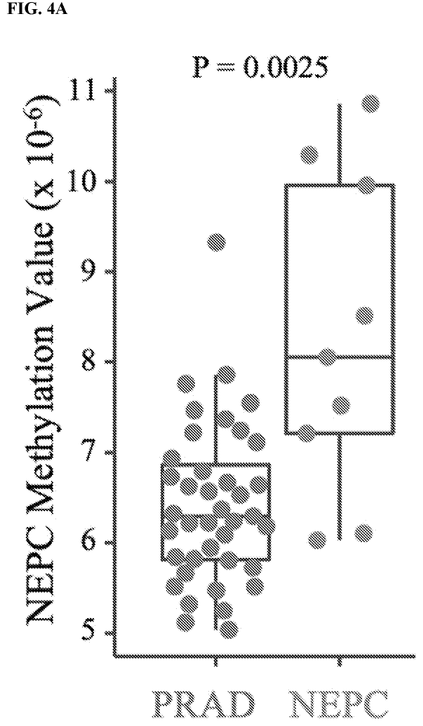

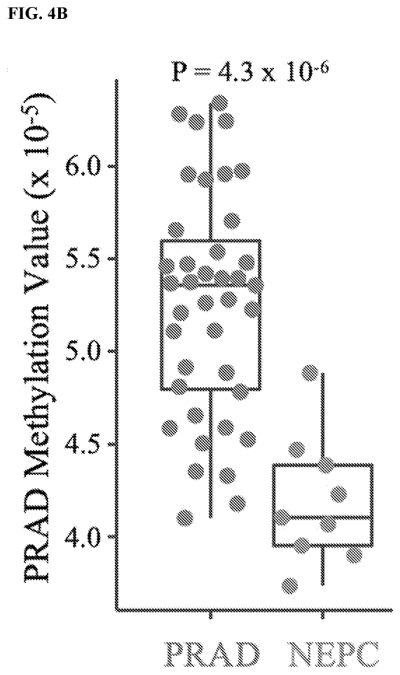

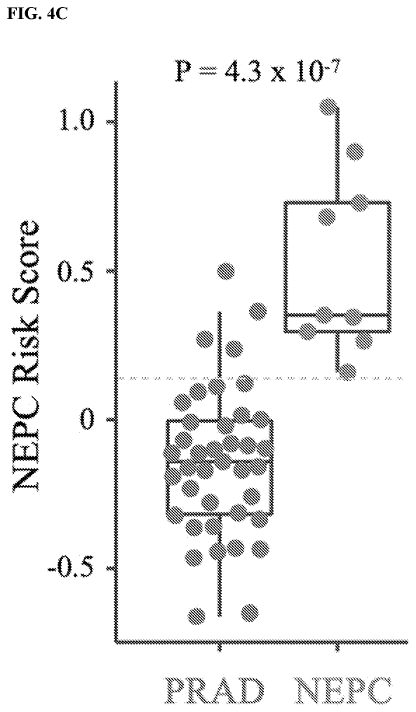

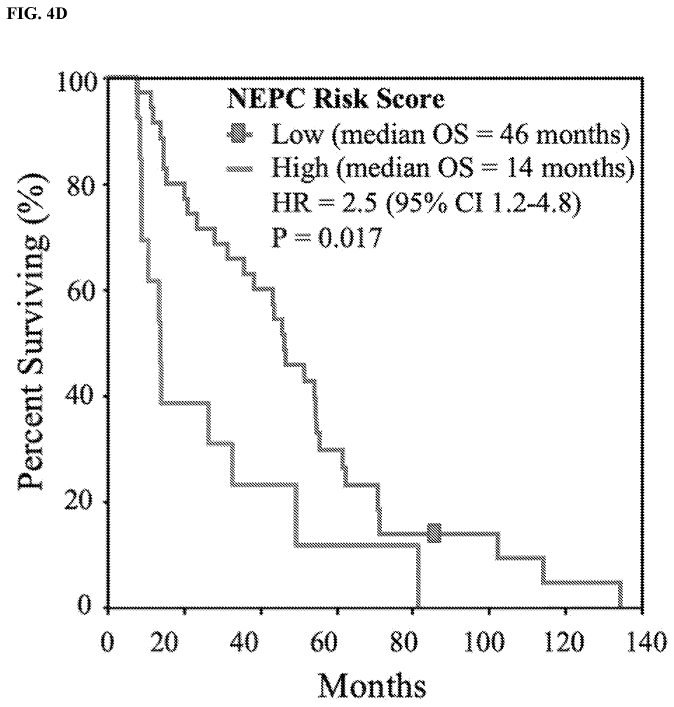

Significantly higher NEPC Methylation Values in men were observed with NEPC than CR-PRAD (median 8.1×10⁻⁶ versus 6.3×10⁻⁶; P=0.0025) (FIG. 4A). In contrast, PRAD Methylation Values were significantly higher in men with CR-PRAD than NEPC (median 5.4×10⁻⁵ versus 4.1×10⁻⁵; P=4.3×10⁻⁶) (FIG. 4B). NEPC Risk Scores were significantly higher in men with NEPC than those with CR-PRAD (median 0.35 versus −0.14; P=4.3×10⁻⁷) (FIG. 4C). The AUROC for accurate classification of men with NEPC versus CR-PRAD based on NEPC Risk Score was 0.96. The optimal NEPC Risk Score cutoff (high >0.15 versus low ≤0.15) demonstrated 100% sensitivity and 90% specificity for detecting NEPC. Further, high versus low NEPC Risk Score was associated with significantly shorter OS from the time of metastases (hazard ratio [HR]=2.5; 95% confidence interval [95% CI]=1.2−4.8; P=0.017) (FIG. 4D). Median OS was 32 months shorter for men with high (14 months) versus low (46 months) NEPC Risk Scores.

### Example 7: Classification of NEPC and CR-PRAD Samples in an Independent cfDNA Validation Cohort (Calculation and Generation of NEPC Risk Value Cut-Offs)

To assess the reproducibility of this approach and the NEPC Risk Score cutoff, an independent multi-institutional validation cohort of plasma samples from 73 men with mCRPC at Dana-Farber Cancer Institute (DFCI) and Weill Cornell Medicine (WCM) were identified, including 16 men with NEPC and 57 with CR-PRAD. cfDNA LPWGS identified tumor DNA in 53 (73%) of samples including 12 (75%) and 48 (72%) of NEPC and CR-PRAD patients, respectively. Samples with cfDNA tumor content <3% were excluded from the cfDNA methylation analysis (FIG. 9). Median cfDNA tumor content was 23% for NEPC patients (range 3.4-43%) and 16% for CR-PRAD patients (range 3.8-39%) (P=0.49) in the test cohort (FIG. 10; Table 9). Median PSA was 0.33 (range 0.01-6.23) versus 112 (4.5-1821) in men with NEPC versus CR-PRAD. Differences between men with NEPC and CR-PRAD in the cfDNA validation cohort were analogous to those observed in the cfDNA test cohort (Table 9).

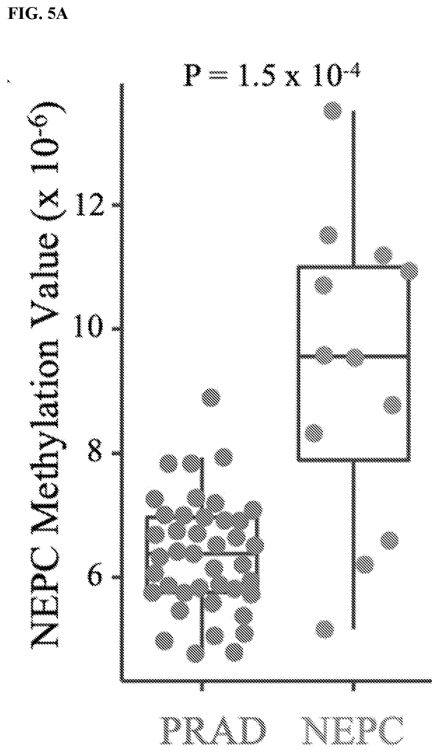

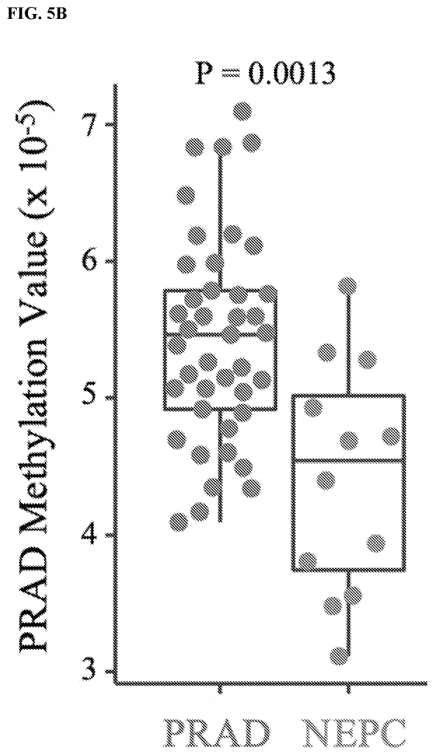

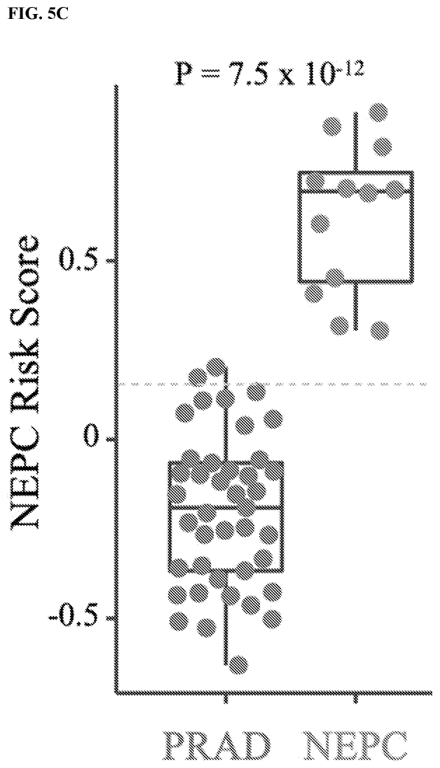

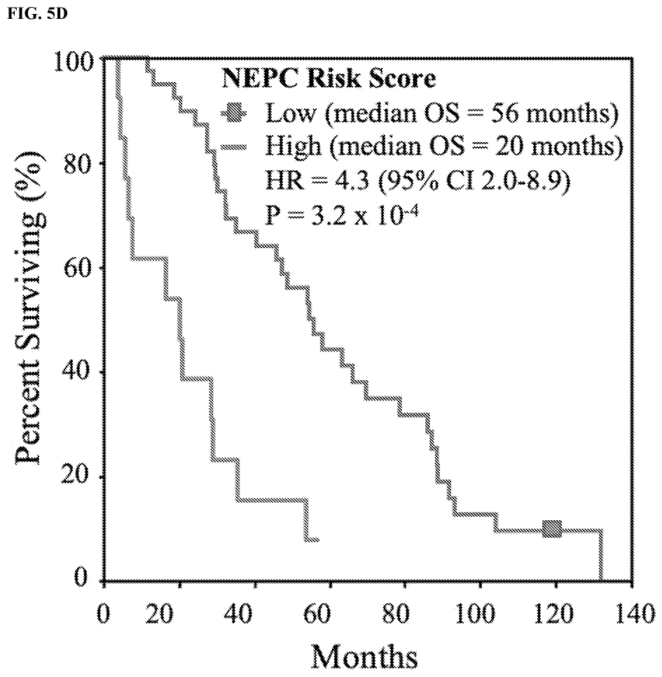

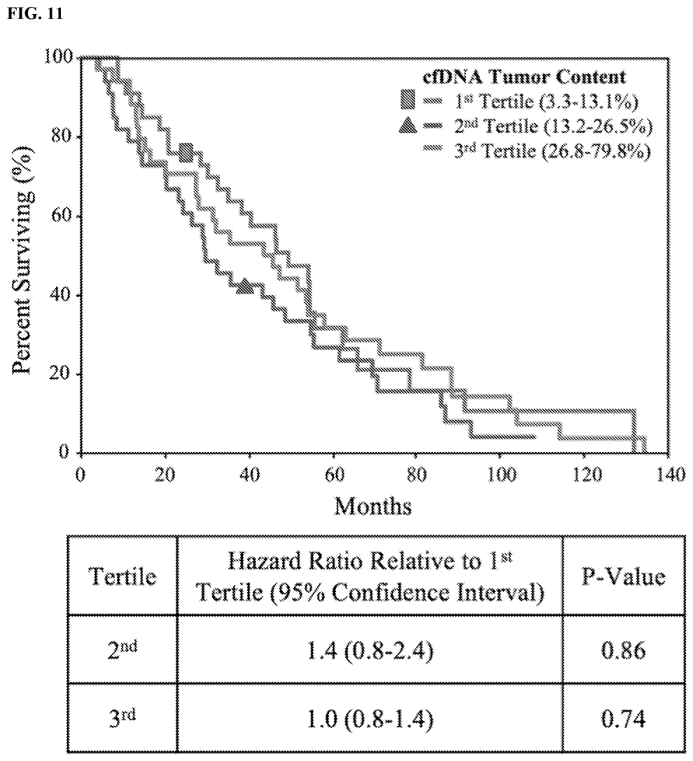

As was observed in the test cohort, NEPC samples in the cfDNA validation cohort exhibited significantly higher NEPC Methylation Values (median 9.6×10⁻⁶ versus 6.4×10⁻⁶; P=1.5×10 4), lower PRAD Methylation Values (median 4.5×10⁻⁵ versus 5.5×1 0-5; P=0.0013), and higher NEPC Risk Scores (median 0.69 versus −0.19; P=7.5×10⁻¹²) than those with CR-PRAD (FIGS. 5A-C). The AUROC for accurate classification of men with NEPC versus CR-PRAD based on NEPC Risk Score was 1.00. Applying the NEPC Risk Score cutoff derived in the test cohort (high >0.15 versus low ≤0.15) to the cfDNA validation cohort resulted in 100% sensitivity and 95% specificity for detecting NEPC. High versus low NEPC Risk Score was associated with significantly shorter OS from the time of metastases (HR=4.3, 95% CI=2.9−8.9; P=3.2×10⁻⁴) (FIG. 5D). Median OS was 36 months shorter for men with high (20 months) versus low (56 months) NEPC Risk Scores. Notably, there was no association of cfDNA tumor content with OS across the two cfDNA cohorts (FIG. 11), suggesting that the negative correlation between NEPC Risk Score and OS is driven by different tumor biology and not higher disease burden.

### Example 8: Patient Vignettes Highlight NEPC Risk Factors in Misclassified CR-PRAD Samples (Calculation and Generation of of NEPC Risk Value Cut-Offs)

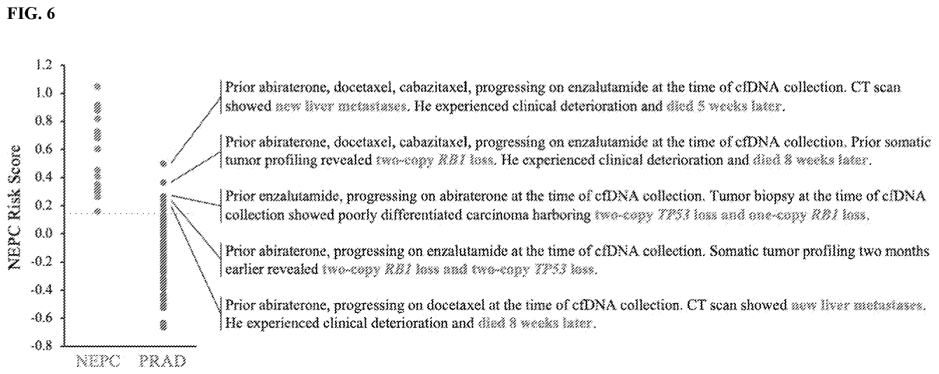

To understand potential factors driving misclassification, medical histories for the six patients with CR-PRAD with NEPC Risk Scores >0.15 across the two cfDNA cohorts were reviewed. Five of these patients had clinical, radiographic, and genomic features associated with NEPC (FIG. 6). The two patients with the highest NEPC Risk Score (0.50 and 0.36) both previously received abiraterone, docetaxel, cabazitaxel, and were on enzalutamide at the time of cfDNA collection. The first patient's CT scan six days after cfDNA collection showed marked increase in metastatic tumor burden, including new liver metastases. He subsequently experienced clinical deterioration and died five weeks later. The second patient previously underwent somatic tumor profiling revealing two-copy RB1 deletion. He experienced clinical deterioration and died 8 weeks after cfDNA collection. The next three patients had all received prior abiraterone and/or enzalutamide. The first (NEPC Risk Score of 0.27) was progressing on abiraterone and underwent tumor biopsy at the time of cfDNA collection showing poorly differentiated carcinoma harboring single-copy RB1 loss and two deleterious TP53 alterations. The second patient (NEPC Risk Score of 0.24) previously received abiraterone and was progressing on enzalutamide at the time of cfDNA collection. Genomic profiling two months earlier showed that the patient's tumor harbored biallelic loss of RB1 and TP53. The third patient (NEPC Risk Score of 0.20) previously received abiraterone and at the time of cfDNA collection was progressing on docetaxel with CT scan showing new liver metastases. He experienced clinical deterioration and died two months later. These hypothesis-generating vignettes suggest the possibility that the cfDNA NEPC Risk Score may identify occult NEPC not detected through routine clinical care.

### Example 9: Association of the Plasma cfDNA Methylome with NEPC Risk Score and Tumor Content (Calculation and Generation of NEPC Risk Value Cut-Offs)

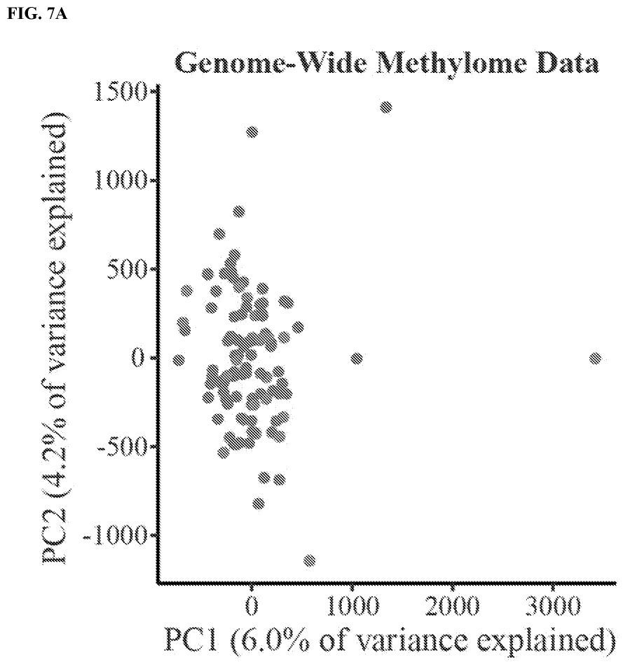

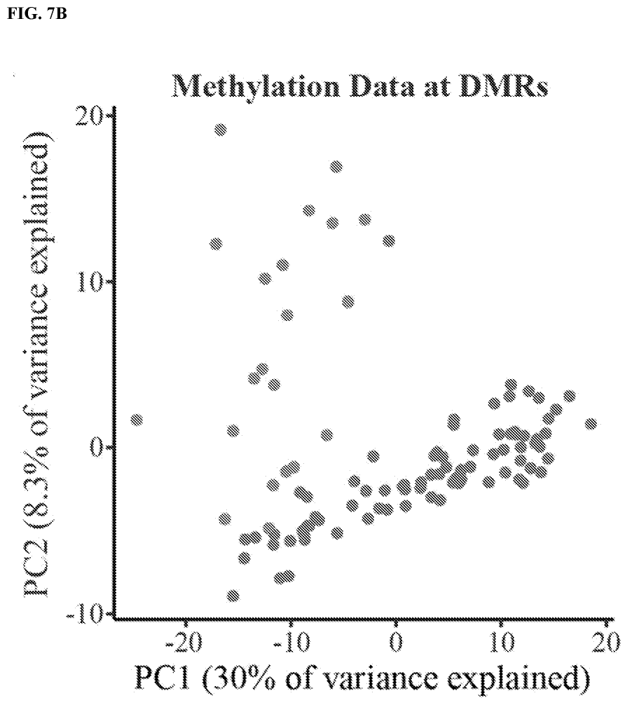

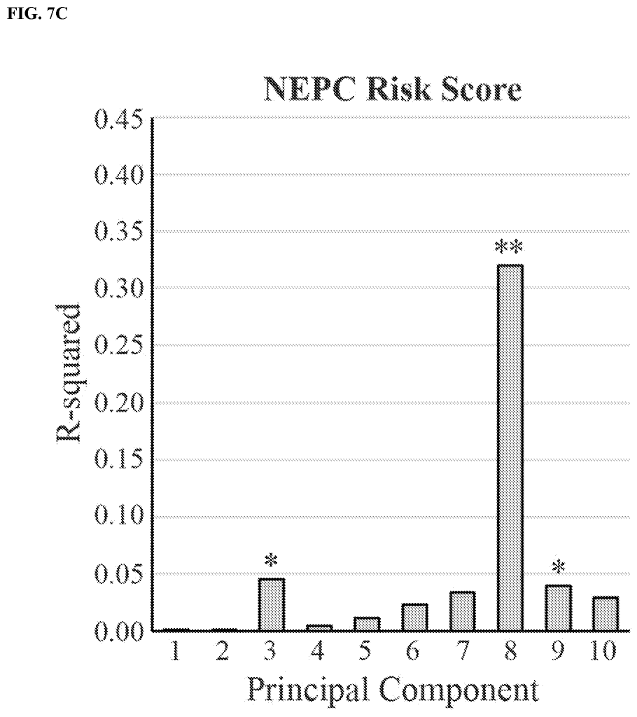

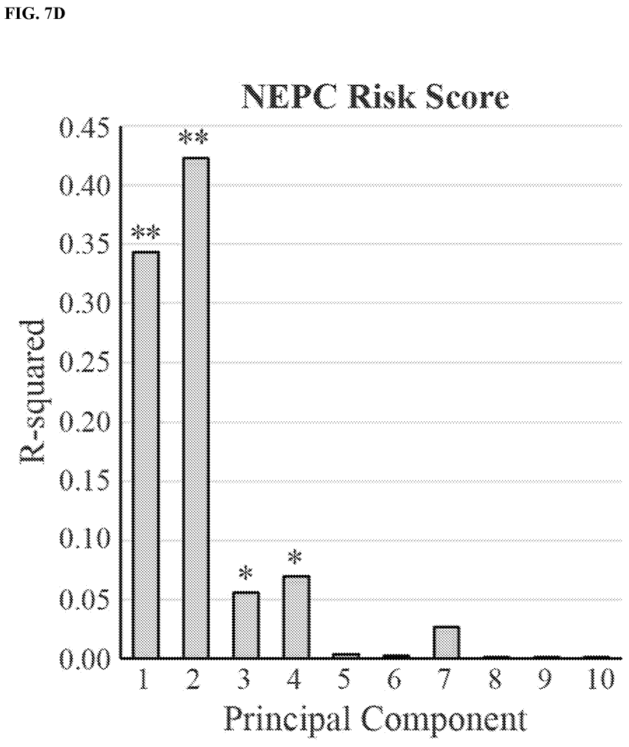

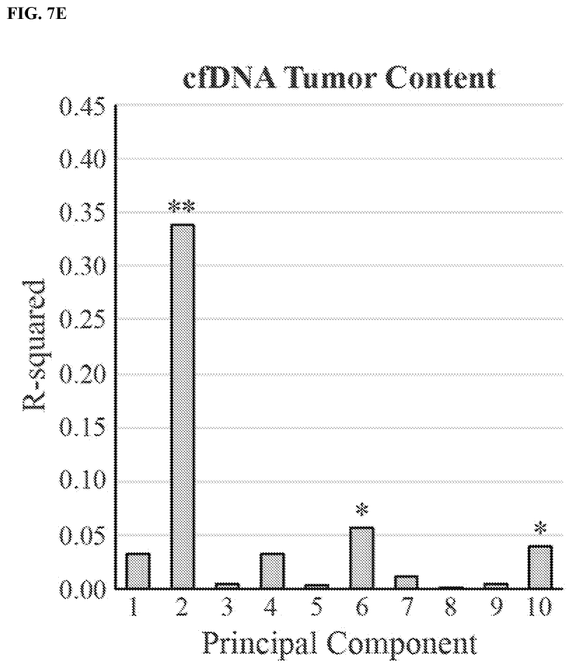

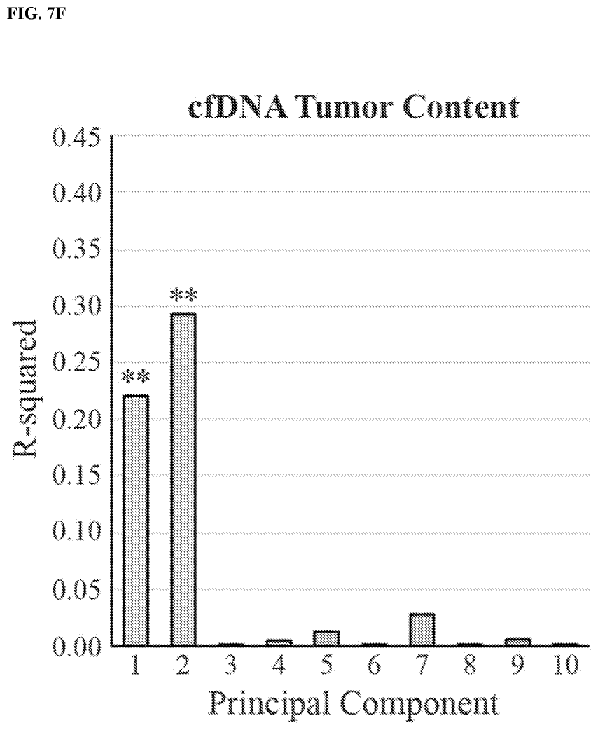

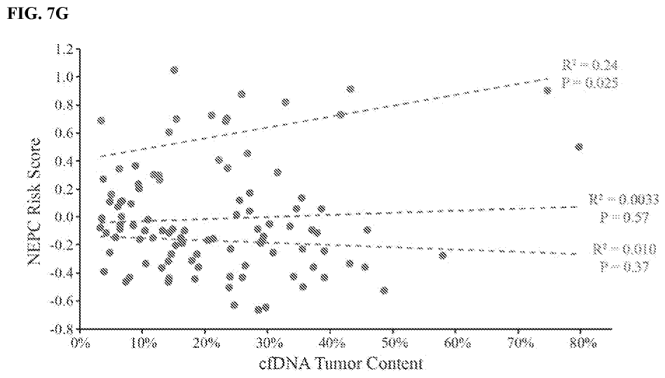

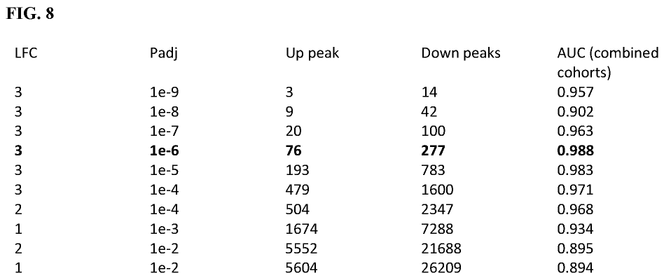

Plasma cfDNA methylome strongly correlates with tumor content in men with metastatic prostate cancer (Wu A, et al. (2020) J Clin Invest. American Society for Clinical Investigation; 130:1991-2000). As such, the association of cfDNA tumor content with the methylome in this cohort and NEPC Risk Scores was investigated herein. Principal component analysis (PCA) of the genome-wide methylome data was preformed (FIG. 7A) and the methylation data at the NEPC- and PRAD-enriched DMRs included in the NEPC Risk Score (FIG. 7B) for the 101 cfDNA samples included in the NEPC Risk Score analyses. In the genome-wide data, the first principal component (PC1) is driven by an outlier sample with high CpG enrichment relative to the others. There was otherwise no separation of NEPC and CR-PRAD samples in PC1 and PC2 in the genome-wide methylome data (FIG. 7A). However, at the DMR sites, PC1 and PC2 clearly separated NEPC and CR-PRAD samples (FIG. 7B).

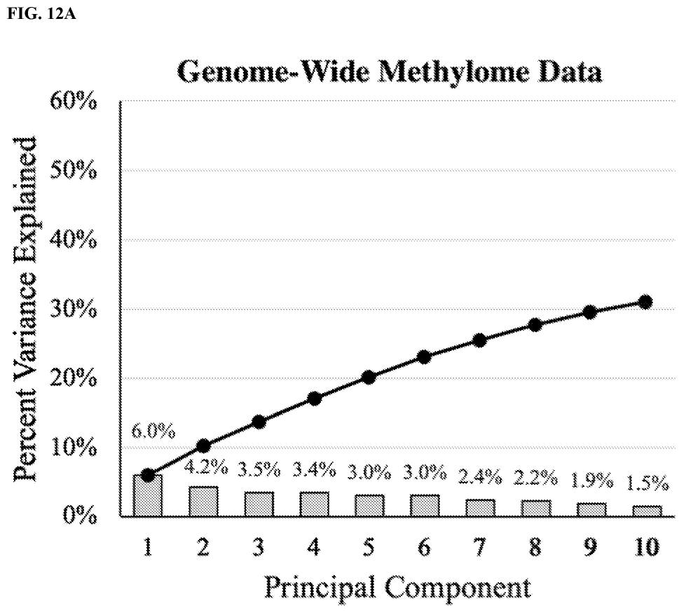

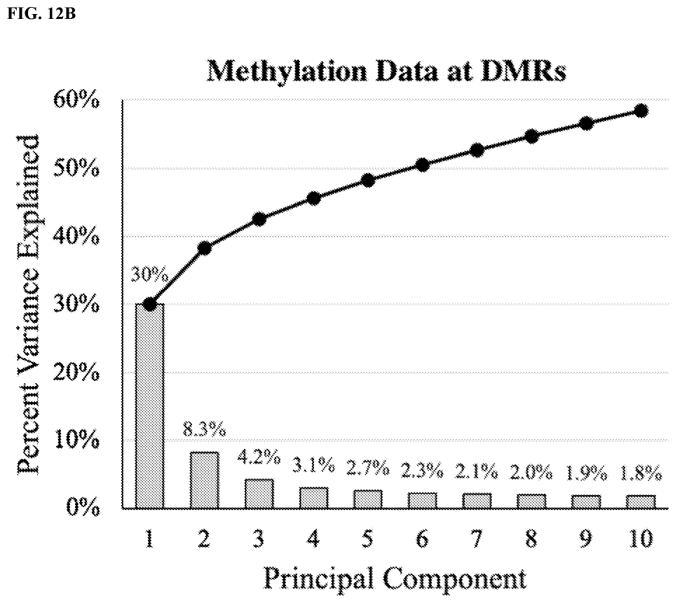

The correlation between each of the first 10 PCs with NEPC Risk Score and cfDNA tumor content was qualified nect. For the genome-wide data, not until PC8, which explained 2.2% of variance, was there a robust correlation with NEPC Risk Score (R²=0.32; P=7.3×10⁻¹) (FIG. 7C; FIG. 12A). In contrast, when limiting to the DMRs included in the NEPC Risk Score, PC1 (R²=0.34; P=1.2×10⁻¹), which explained 30% of variance, and PC2 (R²=0.42; P=2.0×10⁻¹³), which explained 8.3% of variance, both demonstrated robust correlation with NEPC Risk Score (FIG. 7D; FIG. 12B). The correlation between the top PCs and cfDNA tumor content was investigated next. In the genome-wide methylome data, PC2, which explained 4.2% of variance, correlated with cfDNA tumor content (R²=0.34; P=1.7×10⁻¹⁰) (FIG. 7E; FIG. 12A). This result affirms the prior finding that the prostate cancer plasma cfDNA methylome correlates with cfDNA tumor content (Wu A., et al. (2020) J Clin Invest. American Society for Clinical Investigation; 130:1991-2000). When limiting to the DMRs included in the NEPC Risk Score, PC1 (R²=0.22; P=7.2×10⁻⁷) and PC2 (R²=0.29; P=5.1×10⁻⁹) correlated with cfDNA tumor content (FIG. 7F; FIG. 12B). Finally, the correlation between NEPC Risk Score and cfDNA tumor content was assessed (FIG. 7G). Across all NEPC and CR-PRAD cfDNA samples, there was no correlation between NEPC Risk Score and tumor content (R²=0.0033; P=0.57); there was also no correlation in the PRAD samples (R²=0.010; P=0.37). NEPC Risk Score and tumor content did significantly correlate in the cfDNA samples from men with NEPC (R²=0.24; P=0.025). Given this association, suggesting lower NEPC Risk Scores in men with lower cfDNA tumor content, the diagnostic performance of the NEPC Risk Score in the NEPC and CR-PRAD samples across the two cohorts with cfDNA tumor content <10% was evaluated. The NEPC Risk Score in these patients resulted in an AUROC of 0.93; applying the NEPC Risk Score cutoff of 0.15 resulted in 100% sensitivity and 82% specificity for detecting NEPC.

Example 10 follows, and is taken from Chavez et al. Genome Res. (2010) 20: 1441-1450.

### Example 10: Supplementary Methods for Computational Analysis of Genome-Wide DNA-m Ethylation During the Differentiation of Human Embryonic Stem Cells Along the Endodermal Lineage”—Chavez et al., Genome Research 2010

1. MEDIPS package overview

The MEDIPS software was developed for analyzing data derived from methylated DNA immunoprecipitation (MeDIP) experiments (Weber et al. 2005) followed by sequencing (MeDIP-seq). Nevertheless, functionalities like the saturation analysis may be applied to other types of sequencing data (e.g. ChIP-Seq). MEDIPS addresses several aspects in the context of MeDIP-seq data analysis. These are:
estimating the reproducibility for obtaining full genome methylation profiles with respect to the total number of given short reads and to the size of the reference genome,
analyzing the coverage of genome wide DNA sequence patterns (e.g. CpGs) with the given set of sequence reads,
calculating a CpG enrichment factor as a quality control for the immunoprecipitation and for a rough impression of the overall amount of enriched methylated CpGs,
calculating genome wide MeDIP-seq signal densities at a user specified resolution,
calculating genome wide sequence pattern densities (e.g. CpGs) at a user specified resolution,
plotting of calibration plots as a data quality check and for a visual inspection of the dependency between local sequence pattern (e.g. CpG) densities and MeDIP-seq signals,
normalization of MeDIP-seq data with respect to local sequence pattern (e.g. CpG) densities,
summarized methylation values for genome wide windows of a specified length or for user supplied regions of interest (ROIs),
identification of differentially methylated regions on raw or normalized data comparing two sets of MeDIP-seq data and with respect to background data derived from input experiments,
exporting raw and normalized data for visualization in common genome browsers (e.g. the UCSC genome browser (Kuhn et al. 2009)).

The input to MEDIPS is the result of the sequence mapping. MEDIPS can be applied to any genome of interest. The only limitation to its use, are the available genomes within Bioconductors (Gentleman et al. 2004) BSgenome (Pages) package. For a detailed description of the MEDIPS package, please see the tutorial as provided together with the package.

### 2. Modelling of MeDIP-Seq Data

### 2.1 Genome Vector

In order to calculate the genome-wide short read coverage, a targeted data resolution has to be determined. In principle, a short read coverage can be calculated for each base position. Because the resolution of MeDIP-seq data is restricted by the size of the sonicated DNA fragments after amplification and size selection (typically between 0.2-1 kb), a bin size of 50 bp is considered as a reasonable compromise on data resolution and computational costs. Moreover, short reads generated by modern-day sequencers do not represent the full DNA fragments but are of shorter length (e.g. 36 bp). Therefore, the data is smoothed by extending each read to a length according to the estimated average length of sequenced DNA fragments (here 400 bp), either along the + or along the − direction, as specified by the short read dependent strand information. MEDIPS divides each chromosome into bins of size 50 bp and subsequently calculates the short read coverage on this resolution. In the following, the bin representation of the genome is called the genome vector.

### 2.2 Reads Per Million (Rpm)

For each pre-defined genomic bin, the genome vector stores the number of provided overlapping extended short reads (these are the raw MeDIP-seq signals). Based on the total number of provided short reads (n), the raw MeDIP-seq signals can be transformed into a reads per million (rpm) format in order to assure that coverage profiles derived from different biological samples are comparable, although generated from differing amounts of short reads. Let x_{bin}_{ᵢ}, be the raw MeDIP-seq signal of the genomic bin i, where i=1, . . . , m and m is the total number of genomic bins, then the rpm value of the genomic bin is simply defined as:

$${rpm}_{{bin}_{i}} = \frac{{x}_{{bin}_{i}} \cdot 10 ‘}{n}$$

MEDIPS allows for exporting WIG files containing genome wide rpm values at a user-specified resolution (here 50 bp). By utilizing these WIG files, the rpm profiles of the processed biological sample can be immediately visualised using a suitable genome browser.

### 2.3 Quality Controls

### 2.3.1 Saturation Analysis

MeDIP-seq aims to reconstruct methylation profiles on the basis of local short read coverages. It is supposed that an insufficient number of short reads will not represent the true methylation profile. Only when a sufficient number of short reads is generated, the resulting genome vector will represent a saturated methylation profile. Therefore, the saturation analysis addresses the question, whether the number of available short reads is sufficient to generate a saturated and reproducible methylation profile of the reference genome.

The basic assumption of the saturation analysis is that only a sufficient number of short reads will result in a genome wide methylation profile which will be reproducible by another independent set of a similar number of short reads. The correlation of two independently generated genome vectors will increase when the total number of short-reads considered for the construction of each of the two genome vectors increases. It is supposed that the increase of correlation between two independently generated genome vectors will saturate as soon as the total number of considered short reads is increased to a level that is able to represent the analysed methylome in a saturated way. Obviously, the number of short reads that have to be generated for a sufficient sequencing depth depends on the size of the reference genome.

For the saturation analysis, the total set of available regions (n) is divided into two distinct random sets A and B of equal size. Both sets A and B are again divided into k random subsets of equal size:

A=a₁, . . . ,aₖ

B=b₁, . . . ,bₖ

The saturation analysis runs in k iterations. For each set A and B independently, the saturation analysis iteratively selects an increasing number of subsets and creates according genome vectors by using an arbitrary bin size (here 50 bp) and by previously extending the short reads to a suitable length (here 400 bp). In each iteration step, the resulting genome vectors for the subsets of A and B are compared using Pearson correlation. As the number of considered short reads increases during each iteration step, it is supposed that the resulting genome vectors become more similar, a dependency that is expressed by an increased correlation. By storing the resulting correlation coefficients after each iteration step, the change of correlation during the k iteration steps can be visualized by plotting the number of considered reads against the resulting correlation coefficients. Such a plot allows for gaining an impression of the reproducibility of constructing a methylome with respect to the number of considered short reads and with respect to the size of the reference genome.

However, such a saturation analysis can be performed on two independent sets of short reads, only. Therefore, a true saturation analysis can only be calculated for half of the available short reads. Obviously, it is of interest to examine the reproducibility of the MeDIP-seq experiment for the total amount of available short reads. Therefore, the saturation analysis is followed by an estimated saturation analysis. For the estimated saturation analysis, the full set of given regions (n) is artificially doubled by considering each region twice. Afterwards, the described saturation analysis is performed on the artificially doubled set of regions. Because the artificially doubled set of short reads does not represent a true outcome of a MeDIP-seq experiment, the calculated correlations will overestimate the true reproducibility. It is assumed that the true correlation for the full set of available short reads will be between the results of the true and of the estimated saturation analysis. Methods that randomly select data entries can be processed several times in order to obtain more stable results. Therefore, the random partitioning of the short reads into the several subsets of A and B was repeated ten times and the results were averaged.

### 2.3.2 Coverage Analysis

The coverage analysis addresses the question about the genome wide depth of sequence pattern (here CpG) coverage by an increasing number of integrated sequencing derived short reads. For this, all genomic coordinates of the sequence pattern of interest have to be identified. The MEDIPS package provides a function for identifying the genomic positions of arbitrary sequence patterns. In the following, it is expected that all genomic pattern positions are stored on a vector P=p 1, . . . , pᵢ, . . . , pₘ where m is the number of sequence patterns present in the reference genome. For the coverage analysis, the total set of available short reads (A) is divided into k random subsets of equal size:

A=a₁, . . . ,aₖ

The coverage analysis runs in k iterations. The coverage analysis iteratively selects an increasing number of subsets and tests how many pattern positions from P are covered by the available regions. In addition, the coverage analysis counts how many pᵢ's are covered at least Q times, where Q=q₁, . . . , q₁ represents an arbitrary number of coverage depths to be tested. For example, the according function of the MEDIPS package tests by default how many CpGs are covered at least 1×, 2×, 3×, 4×, 5×, and 10× times (this is equivalent to the notation Q=1, 2, 3, 4, 5, 10). The k-th iteration step of the coverage analysis shows the depth of sequence pattern coverages obtained with the full set of available short reads.

The advantage of the iterative approach is that the behaviour of pattern coverage can be examined with respect to an increasing number of considered short reads. For this, coverage curves can be generated by plotting the number of covered sequence patterns after each iteration step and for each level of Q against the number of considered short reads. The progressions of the resulting coverage curves indicate the state of saturation of the overall sequence pattern coverages. Because methods that randomly select data entries can be processed several times in order to obtain more stable results, the random partitioning of the short reads into the several subsets of A was repeated ten times and the results were averaged. As for calculating the genome vector and as done for the saturation analysis the length of the short reads were previously extended to 400 bp.

### 2.3.3 CpG Enrichment

As a third MeDIP-seq data quality control, the CpG enrichment approach examines how strong the genomic regions underlying the obtained short reads are enriched for CpGs compared to the frequency of CpGs present in the reference genome. For this, firstly the number of cytosines (G.c), the number of guanines (G.g), the number CpGs (G.cg), and the total number of bases (m) within the specified reference genome (here hg19) are counted. Subsequently, the relative frequency of CpGs and the observed/expected (Gardiner-Garden and Frommer 1987) ratio of CpGs as present in the reference genome are calculated as:

$$\begin{matrix}Genome . {CpG}_{rel . f} = \frac{G . cg}{m} \\ Genome . {CpG}_{obs / exp} = \begin{matrix}G . cg \cdot m \\ G . c \cdot G . g\end{matrix}\end{matrix}$$

Additionally, the number of cytosines (SR.c), the number of guanines (SR.g), the number CpGs (SR.cg), and the total number of bases (n) are counted for the DNA sequences underlying the given short reads. Subsequently, the relative frequency of CpGs and the observed/expected ratio of CpGs as present in the short reads specific DNA sequences are calculated accordingly:

$$\begin{matrix}SR . Cp {G}_{rel . f} = \frac{S R . c g}{n} \\ SR . {CpG}_{obs / exp} = \frac{S R . cg \cdot n}{S R . c \cdot SR . g}\end{matrix}$$

The final enrichment values result by dividing the relative frequency of CpGs (or the observed/expected value, respectively) of the short reads by the relative frequency of CpGs (or the observed/expected value, respectively) of the reference genome:

$$\begin{matrix}{enrich}_{rel , f} \frac{SR . {CpG}_{rel , f}}{Genome . Cp {G}_{rel . f}} \\ {enrich}_{obs / exp} = \frac{SR . {CpG}_{obs / exp}}{Genome . {CpG}_{obs / exp}}\end{matrix}$$

For short reads derived from an INPUT experiment (that is sequencing of none-enriched DNA fragments), the enrichment values are expected to be close to 1. In contrast, short reads derived from MeDIP-seq experiments are expected to be enriched for CpG rich DNA sequences, a circumstance which will be indicated by increased enrichment scores.

### 2.4 MeDIP-Seq Data Normalization

The idea of a MeDIP experiment is to identify cytosine methylation profiles of a sample of interest by immunocapturing methylated CpGs (mCpGs) using an mCpG specific antibody (Weber et al. 2005). However, it has been shown (Down et al. 2008; Pelizzola et al. 2008) that MeDIP signals scale with local densities of CpGs and are not necessarily influenced by mCpGs, only. Therefore, the need for MeDIP-seq data correction occurs through an unspecific binding of the utilized antibody to un-methylated CpGs, especially in genomic regions associated to elevated densities of un-methylated CpGs and low densities of mCpGs.

### 2.4.1 Coupling Factors

Similar to other MeDIP normalization approaches (Down et al. 2008; Pelizzola et al. 2008), the presented method corrects for the unspecific antibody binding by incorporating local CpG densities into the MeDIP-seq derived signals. In order to integrate the information about CpG densities into the following analysis, it is necessary to identify the genomic positions of all CpGs. This can be achieved by executing the MEDIPS.getPosition( ) function of the MEDIPS package. Following the valuable concept of coupling factors presented by Down et al. (Down et al. 2008), a coupling vector is calculated based on the received genomic positions of all CpGs. The coupling vector is of the same size as the predefined genome vector (here bin size of 50 bp) but contains local CpG densities (also called coupling factors) for each genomic bin, instead. For each predefined genomic bin at position b, the density of surrounding CpGs has to be calculated. For this, first a maximal distance (d) has to be defined. Only CpGs within the range of [b−d,b,b+d] will contribute to the final local coupling factor at b. The optimized value for d will reflect the estimated size of the sonicated DNA fragments after amplification and size selection. This is because MeDIP-seq derived signals at position b are influenced by sequenced DNA fragments that overlap with position b. Immunoprecipitation of these DNA fragments can be caused by a methylated and antibody bound CpG located at any position of the DNA-fragment. The maximal distance of a CpG contributing to the signal at b is therefore the estimated average length of the sonicated DNA fragments (d).

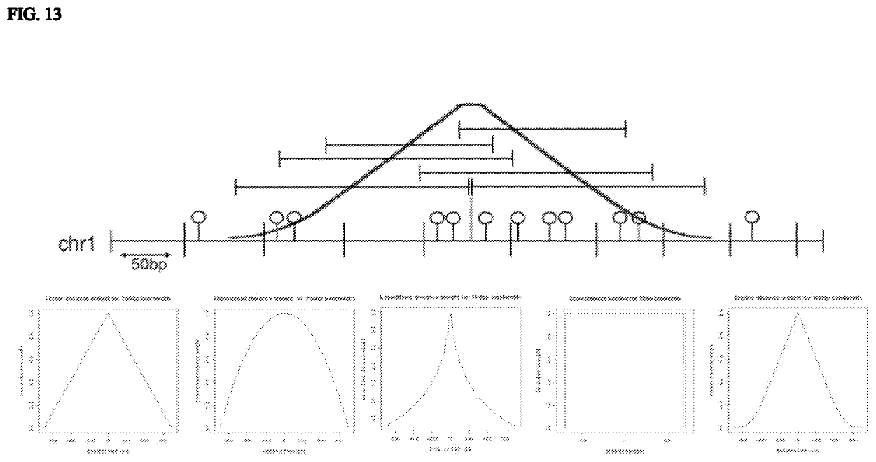

There are several ways for calculating coupling factors for genomic bins. Let c be the chromosomal position of a CpG and as b is the chromosomal position of a genomic bin, dist=|b−c| is the distance between the genomic bin and the CpG. A CpG will contribute to the coupling factor of a genomic bin at position b, if dist≥d. The simplest way is to count the number of CpGs within the maximal distance d around a genomic bin at position b (count function). Another approach is to weight each CpG by its distance to the current genomic bin. CpGs farther away from the current genomic bin will receive smaller weights, whereas CpGs close to the genomic bin will receive higher weights. The upper panel in FIG. 13 illustrates a genome vector generated by defining a bin size of 50 bp. In addition, CpGs are given in a schematic way. The Figure illustrates that immuoprecipitated DNA fragments of an estimated average length greater than the pre-defined bin size can contribute to the signal of the genomic bin at position b (vertical red line). Moreover, the schematic distance function illustrates that CpGs close to position b will receive higher weights than CpGs located farther away. There are several possible ways for defining weighting functions. In the context of this thesis, the following weighting functions were evaluated: count, linear, exp (Pelizzola et al. 2008), log (Pelizzola et al. 2008), and custom (Down et al. 2008). The images at the bottom of FIG. 13 show the progression of these weighting functions by defining a maximal distance d=700.

[symbol]

Whereas the weighting functions count, linear, exp, and log are calculated by defined formulas, the custom function allows for specifying user-defined weights for any possible distance dist. For example, Down et al. (Down et al. 2008) have generated custom weights for the distances dist∈[0,648]. These weights were estimated empirically by sampling from the fragment-length distribution and randomly placing each fragment such that it overlaps the genomic bin Down et al. 2008). Such weights can be up-loaded using MEDIPS and are returned when the custom function is called. Let C_{cb} be the coupling factor between a CpG at position c and a genomic bin at position b calculated based on an arbitrary weighting function and for any specified parameter d. Then

$${C}_{tot} = {\sum }_{c} {C}_{c b}$$

is the sum of coupling factors at the genomic bin b with respect to all CpGs at a genomic position c, where |b−c|≤d. For simplification, in the following, Cᵢₒₜ is called the coupling factor at a genomic bin b and gives a measure of local CpG density.

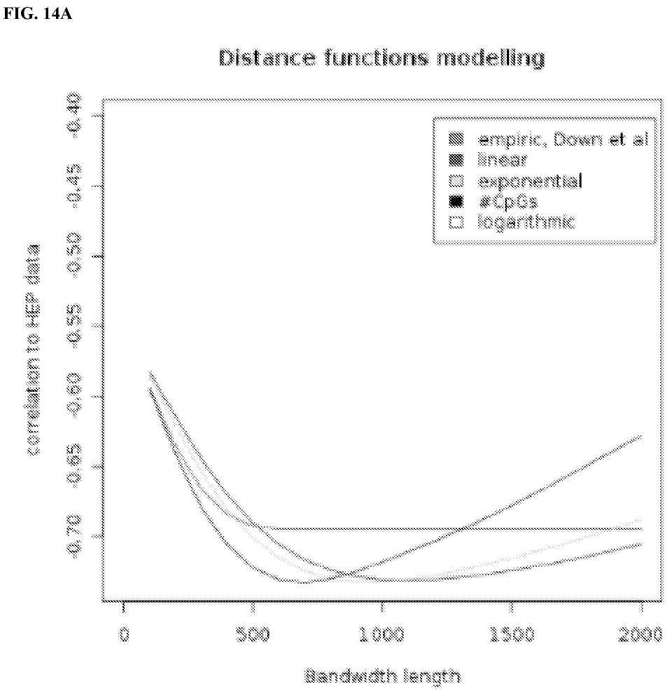

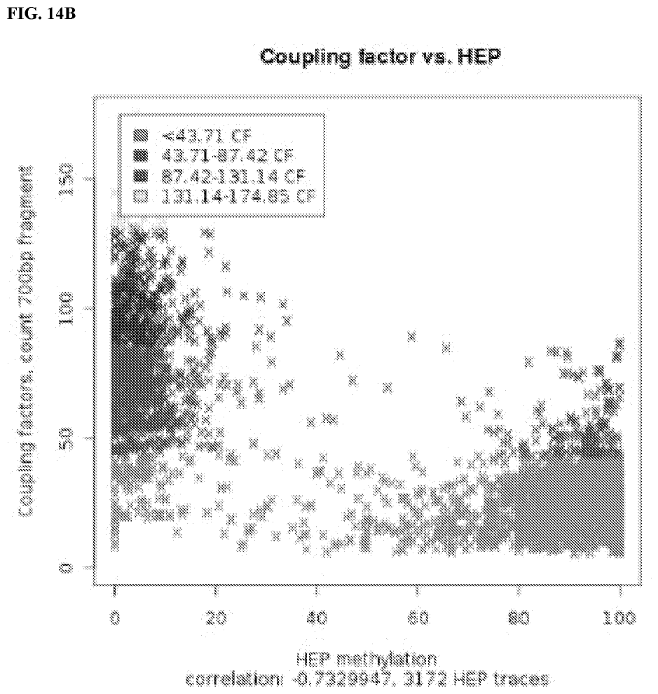

It has been shown Weber et al. 2005; Eckhardt et al. 2006) that in mammalian cells, methylation is negatively correlated to CpG densities. In other words, regions of low CpG density tend to be high methylated, whereas regions of high CpG density tend to be mainly unmethylated. In order to test the correlation of measured methylation values (Eckhardt et al. 2006) compared to local CpG densities calculated with respect to the different weighting functions, we have systematically calculated coupling vectors (bin size=50) with varying d∈[0, 2000] using the weighting functions count, linear, exp, log, as well as for the empirically derived weights presented by Down et al. (Down et al. 2008) (custom). Because the custom weights are available for the range d∈[0, 64 8], only, the weight at d=648 is also utilized for the remaining distances up to d=2000. For the comparisons, we have accessed DNA-methylation values derived from bisulphite sequencing experiments of a sperm sample as presented by the human epigenome project (HEP) (Eckhardt et al. 2006). Bisulphite sequencing derived methylation data was generated for approximately 3000 selected genomic regions (called HEP traces) of length 50 bp to 500 bp (Eckhardt et al. 2006). In order to compare CpG densities to the available methylation data, for all utilized weighting functions with varying parameter d, we have calculated mean coupling factors for each of the HEP traces and examined the relation to corresponding mean methylation values by Pearson correlation. FIG. 14 shows the resulting Pearson correlations for varying parameter d and for the several tested weighting functions.

Interestingly, the best negative correlation (that is the higher the CpG density, the lower the bisulphite derived methylation values) was achieved (−0.73) by setting the parameter d=700 and by using the count function. For this parameter settings, FIG. 14 shows a scatterplot comparing mean HEP methylation values and mean coupling factors. Here, each data point represents a HEP trace and the plot contrasts the mean methylation value (x-axis) with the mean CpG denisty (y-axis). The color code divides the full range of CpG densities into quantiles. Based on these results, in the following, the coupling vector is calculated by specifying d=700 and by using the count function. However, the MEDIPS package allows for justifying the according parameters or for supplying any custom defined distance weights. Moreover, coupling vectors can be calculated for any arbitrary DNA sequence pattern and the resulting coupling vectors can be exported into a WIG file for visualizing the sequence pattern densities along the chromosomes using a suitable genome browser.

### 2.4.2 Calibration Curve

As we have created a genome vector that contains the raw signals at each genomic bin as well as an according coupling vector containing the calculated coupling factors at each genomic bin, the dependency of local MeDIP-seq signal intensities and local CpG densities can be examined. However, by simply plotting the genome vector against the coupling vector, no concrete dependency is observable. However, a dependency between CpG densities and MeDIP-seq signals can be made tangible by calculating the calibration curve (Down et al. 2008). Calculation of the calibration curve is achieved by first dividing the total range of coupling factors into regular levels. Second, all genomic bins are partitioned into these levels by considering their associated coupling factors. Finally, for each level of coupling factors, the mean signal and mean coupling factor of all genomic bins that fall into this level are calculated. As the calibration curve represents the averaged signals and coupling factors over the full range of coupling factors, it reveals the experiment specific dependency between signal intensities and CpG densities. In fact, for the low range of coupling factors, the calibration curve indicates that the MeDIP-seq signals, in average, increase because of an increasing CpG density. Therefore, an increased signal is not necessarily caused by a higher level of mCpGs but scales with the general CpG density. In contrast, for INPUT derived sequencing data this dependency of CpG density and sequencing signals is not observable (see Supplementary FIG. 3c of the main manuscript). Therefore, the calibration plot is very characteristic for MeDIP-seq data and the quality of the enrichment step of the MeDIP experiment can be estimated by visual inspection of the progression of the calibration curve. For higher levels of CpG densities, the mean MeDIP-seq signals decrease. It is assumed that this decrease is caused by the fact that in biological systems, regions of higher CpG densities are mainly unmethylated. Interestingly, in biological systems, cytosine methylation occurs mainly in regions of low CpG density. The other way round, cytosines located in regions of high CpG density are mainly unmethylated. This circumstance implicates that the dependency between increased signal intensities caused by increased CpG densities is visible for regions of low CpG densities, only.

### 2.4.3 Relative and Absolute Methylation Scores

The calibration curve reveals that, in average, an increase of MeDIP-seq signals is caused by an increasing CpG density. This approximately linear dependency is visible for the low range of coupling factors, only. For higher levels of CpG densities, the mean MeDIP-seq signals decrease. As mentioned above, it is assumed that this decrease is caused by the fact that in mammalian cells, regions of higher CpG densities are mainly unmethylated. In agreement with this assumption, Pelizzola and colleagues (Pelizzola et al. 2008) have shown that the dependency of MeDIP derived signals and CpG density continues for higher levels of CpG densities, by analysing artificially fully methylated samples using MeDIP-Chip. In fact, they identified a sigmoidal dependency between CpG density and MeDIP-Chip data (Pelizzola et al. 2008). In agreement with Pelizzola et al. (Pelizzola et al. 2008), the signal plateau in the lower range of chip signals is caused by background noise and it is assumed that the signal plateau in the upper range of chip signals occurs by a saturation of hybridization events and is therefore an array specific artefact.

By visual inspection of the MeDIP-seq derived calibration curves, and motivated by the observations made by Pelizzola et al. (Pelizzola et al. 2008), a continuing linear dependency of MeDIP-seq signals for higher levels of CpG densities is assumed. Analogous to Down et al. (Down et al. 2008), the local maximum of mean MeDIP-seq signals of the calibration curve in the lower part of coupling factors is identified. Let

y=y₁, . . . ,yᵢ

be the mean coupling factors, and let

x=x₁, . . . ,xₗ

be the according mean MeDIP-seq signals of the calibration curve, where l is the number of tested coupling factor levels and i=1, . . . , l, then the smallest level i is identified, where

xᵢ₋₃,xᵢ₋₂,xᵢ₋₁,≤xᵢ≥xᵢ₊₁,xᵢ₊₂,xᵢ₊₃,

Let iₘₐₓ be the according identified level of i, then

yₘₐₓ=y₁, . . . ,yᵢ_{ₘₐₓ}

xₘₐₓ=x₁, . . . ,xᵢ_{ₘₐₓ}

is the part of the calibration curve in the low range of coupling factors, where an approximately linear dependency between MeDIP-seq signals and coupling factors is observed. Here, xₘₐₓ can be explained by a function of yₘₐₓ as

xₘₐₓ=f(yₘₐₓ)+ε

where ε is an error variable (i.e. measurement errors) that is expected to spread by chance and therefore, its expectation value is E(ε)=0. Because a linear dependency between xₘₐₓ and yₘₐₓ is assumed, xₘₐₓ can be described as

xₘₐₓ=α+β·yₘₐₓ+ε

where the parameter α is the theoretical y-intercept, and the parameter β is the theoretical slope. Based on the pre-calculated xₘₐₓ and yₘₐₓ vectors, linear regression is performed, in order to identify a suitable linear model. Linear regression estimates concrete values a and b for the parameters α and β so that it is valid:

xₘₐₓ,=a+b·yₘₐₓ,+eᵢ

where i=1, . . . , iₘₐₓ. Here, the residuum eᵢ reflects the difference between the regression curve a+b·yₘₐₓ_{ᵢ}, and the measurements for xₘₐₓ_{ᵢ}. Moreover, xₘₐₓ_{ᵢ} can be replaced by an estimate \hat{x}ₘₐₓ_{ᵢ}, where xₘₐₓ_{ᵢ} −\hat{x}ₘₐₓ_{ᵢ}=eᵢ and therefore, it is valid:

\hat{x}ₘₐₓ_{ᵢ}=a+b·yₘₐₓ_{ᵢ},

MEDIPS calculates the linear regression model using the least squares approach (www.R-Project.org) and concrete values a and b are obtained. Subsequently, for the low range of coupling factors, the observed progression of the calibration curve can be modelled. As discussed above, a continuing linear dependency between MeDIP-seq signals and CPG density is expected for the higher range of coupling factors. Based on the obtained linear model parameters, concrete \hat{x}ₘₐₓ_{ᵢ} values can be calculated for the full range of coupling factors. Therefore,

\hat{x}=\hat{x}₁, . . . ,\hat{x}ₘₐₓ_{ᵢ}, . . . ,\hat{x}₁

are the estimated mean MeDIP-seq signals over the full range of coupling factor levels l. For MeDIP-seq data normalization, \hat{x}is utilized in order to weight the observed MeDIP-seq signals of the genomic bins with respect to their associated coupling factors. Let (x_{bin}_{ᵢ}, y_{bin}_{ᵢ},) be
the raw MeDIP-seq signal of the genomic bin i (i.e. the [symbol] extended short reads), and the pre-calculated coupling factor at the genomic bin i, where i=1, . . . , m and in is the total number of genomic bins, then the normalized relative methylation score is defined as

$${rms}_{{bin}_{i}} = log 2 ( \frac{{x}_{{bin}_{i}} 10 ‘}{( a + b \cdot {y}_{{bin}_{i}} ) \cdot n} ) = log 2 ( \frac{{x}_{{bin}_{i}} \cdot 10 ‘}{{\hat{x}}_{{bin}_{i}} \cdot n} )$$

where \hat{x}_{bin}_{ᵢ}=a+b·y_{bin}_{ᵢ} is the estimated weighting parameter obtained by considering the coupling factor y_{bin}, of the genomic bin i, and n is the total number of short reads considered for the generation of the genome vector. Based on the total number of short reads (n), the raw MeDIP-seq signals are, in parallel, transformed into a reads per million (rpm) format in order to assure that rms values are comparable between methylomes generated from differing amounts of short reads. The MEDIPS package subsequently transforms the resulting rms data range into the consistent interval [0,1000], before finally returned. We consider the rms values as the normalized MeDIP-seq signals corrected for the effect of unspecific antibody binding.

In order to identify an absolute methylation estimate for any specified region of interest, i.e. either any functional genomic regions like promoters or CpG islands or genome wide windows of arbitrary length, the raw MeDIP-seq values are normalized into absolute methylation scores (ams). The absolute methylation scores correct for the relative CpG density of the regions of interest and therefore, allow for comparing methylation profiles of regions with differing CpG densities. Let ROI=((x_{bin}_{₁}, y_{bin}_{₁}), . . . , (x_{bin}_{_{S}}, y_{bin}_{ₛ})) be the raw MeDIP-seq signals and coupling factors of adjacent genomic bins i that define a region of interest (ROI), where i=1, . . . , s and s is the total number of genomic bins comprised by the ROI, then the absolute methylation score for the ROI is defined as

$${ams}_{ROI} = log 2 ( \frac{\frac{1}{s} {\sum }_{i = 1}^{s} \frac{{x}_{{bin}_{i}} \cdot 10 ‘}{( a + b \cdot {y}_{{bin}_{i}} ) \cdot n}}{\frac{1}{s} {\sum }_{i = 1}^{s} {y}_{{bin}_{i}}} )$$

Again, the MEDIPS package subsequently transforms the resulting ams data range into the consistent interval [0,1000], before finally returned. Analogous to Pelizzola et al. (Pelizzola et al. 2008), we interpret the ams values (Pelizzola et al. (Pelizzola et al. 2008) call them rims), as the measure of the normalized methylation that is independent of the CpG density of the corresponding genomic region.

### 3 Identification of Differentially Methylated Regions (DMRs)

Identification of DMRs is essential for determining local differences in the methylation profiles of diverse biological samples. While there exist several methods for determining statistically significant enriched genomic regions from ChIP-on-Chip (Li et al. 2005; Johnson et al. 2006; Toedling et al. 2007; Chavez et al. 2009) and ChIP-Seq experiments (Boyle et al. 2008; Ji et al. 2008; Valouev et al. 2008; Lun et al. 2009; Rozowsky et al. 2009), the identification of differentially methylated regions from MeDIP-seq data remains insufficiently explored. The main difference between the ChIP-Seq and MeDIP-seq approaches is that TFBSs are of short length (8-16 bp) and therefore, ChIP-Seq specific methods intend to identify isolated short genomic regions of high short read enrichments. In contrast, CpGs are spread more widely along the chromosomes and are partly accumulated in CpG islands of length >300 bp. Moreover, methylation alterations may occur at few CpG locations, only, and therefore, no sharp TFBSs like ChIP-Seq peaks are expected. Subsequently, in order to identify DMRs, comparatively longer genomic stretches have to be considered and methylation alterations have to be determined in a more sensitive way.

For the identification of DMRs, we propose two alternative approaches. Firstly, it is of interest to specify pre-defined genomic regions of interest (ROIs) like CpG islands, promoters etc., and to specifically compare methylation patterns for these regions. Secondly, it is of interest to calculate differential methylation for genome wide frames of arbitrary length. However, in both cases we call any predefined genomic region as ROI. Here, we present a statistical approach for calculating differential methylation for any predefined ROI, based on sequencing data from two different MeDIP treated samples (Control and Treatment) with respect to an additional input sequencing data set (Input). Let C, T, and I be the genome vectors generated based on the sequencing data from Control, Treatment, and Input using an arbitrary bin size b and let ROI be a set of predefined ROIs:

ROI=ROI1, . . . ,ROIᵢ, . . . ,ROIₙ

where n is the number of ROIs to be tested and the ROIᵢ's are of length m₁, . . . mₙ. In the following, the identification of DMRs is only supported for any ROIᵢ of length mᵢ≥5·b. Therefore, each ROIₗ consists out of a set of at least five genomic bins (bin_{ROI}_{ᵢ}), where bin_{ROI}_{ᵢ}=bin_{i,1}, . . . , bin_{i,j}, . . . , bin_{i,k}_{ᵢ}∈ROIᵢ and

$${k}_{i} = floor ( \frac{{m}_{i}}{b} ) .$$

For each ROIᵢ, mean rpm and
[symbol] calculated based on C and T as:

$$\begin{matrix}C . {RPM}_{{ROI}_{i}} = \frac{1}{{k}_{i}} {\sum }_{j = 1}^{{k}_{i}} rpm ( C . {bin}_{i , j} ) \\ C . {RMS}_{{ROI}_{i}} = \frac{1}{{k}_{i}} {\sum }_{j = 1}^{{k}_{i}} rms ( C . {bin}_{i , j} ) \\ T . {RPM}_{{ROI}_{i}} = \frac{1}{{k}_{i}} {\sum }_{j = 1}^{{k}_{i}} rpm ( T . {bin}_{i , j} ) \\ T . {RMS}_{{ROI}_{i}} = \frac{1}{{k}_{i}} {\sum }_{j = 1}^{{k}_{i}} rms ( T . {bin}_{i , j} )\end{matrix}$$

where rpm(C.bin_{i,j}), rms(C.bin_{i,j}), rpm(T.bin_{i,j}), and rms(T.bin_{i,j}) are the pre-calculated rpm (see section 2.2) and rms values (see section 2.4.3) for the according genomic bins of the Control and of the Treatment samples. In addition, for each ROIᵢ, mean rpm values are
calculated based on I as:

$$I . {RPM}_{{ROI}_{i}} = \frac{1}{{k}_{i}} {\sum }_{j = 1}^{{k}_{i}} rpm ( I . {bin}_{i , j} )$$

where rpm(I.bin_{i,j}) are the pre-calculated rpm values for the genomic bins of the Input sample. Based on the mean rms values of the Control and of the Treatment sample, for each ROIᵢ the following ratio is calculated:

$$r . {rms}_{{ROI}_{i}} = \frac{C . {RMS}_{{ROI}_{i}}}{T . {RMS}_{{ROI}_{i}}}$$

In addition, by considering the mean rpm values of the Control or of the Treatment sample, respectively, the following ratios are calculated with respect to rpm values of the Input sample:

$$\begin{matrix}r . rpm . {C}_{ROI} = \frac{C . RP {M}_{{ROI}_{i}}}{I . R P {M}_{{ROI}_{i}}} \\ r . rpm . {T}_{{ROI}_{i}} = \frac{T . {RPM}_{{ROI}_{i}}}{I . RP {M}_{{ROI}_{i}}}\end{matrix}$$

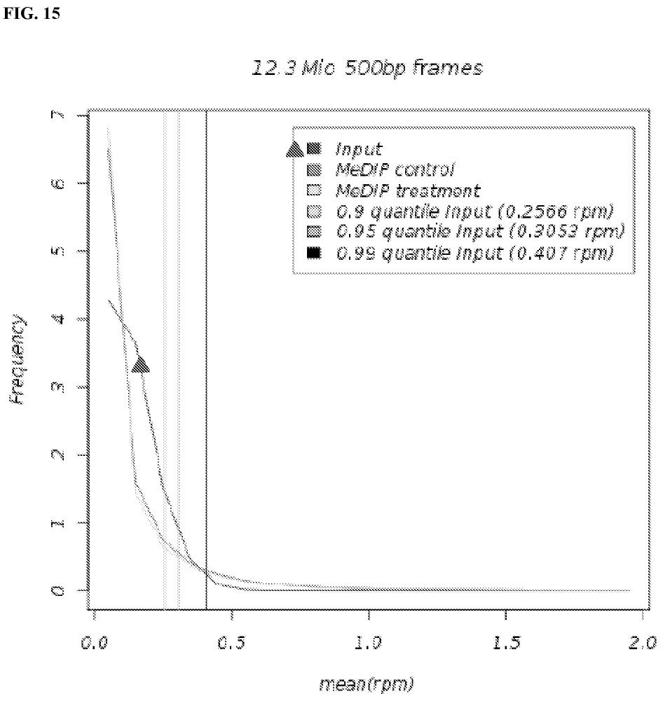

Because local background sequencing signals are variable along the chromosomes due to differing DNA availability, a global background rpm signal threshold is estimated based on the distribution of all calculated I.RPM_{ROI}_{ᵢ} values. This is done by defining a targeted quantile qt (e.g. qt=0.95) and by identifying the I.RPM_{ROI}_{ᵢ} value (t), where qt % of all I.RPM_{ROI}_{ᵢ} values are <t. FIG. 15 illustrates the distributions of the I,RPM_{ROI}_{ᵢ} C.RPM_{ROI}_{ᵢ}, and T.RPM_{ROI}_{ᵢ} values as obtained from the Input, hESCs (Control) and DE (Treatment) samples, when defining regions of interest as overlapping genome wide 500 bp windows, where neighbouring windows overlap by 250 bp. By setting the qt parameter to qt=1.90, here, an rpm threshold t=0.2566 is obtained from the input I.RPM_{ROI}_{ᵢ} distribution.

[symbol]

This estimated global minimal mean rpm threshold t will serve as an additional parameter for selecting genomic regions that show mean MeDIP-seq derived rpm signals of at least t in either the Control or the Treatment sample.

Moreover, statistical testing is utilized in order to rate whether the obtained rms data series of the genomic bins within any ROIᵢ significantly differs in the Control sample compared to the Treatment sample. For each ROIᵢ it is tested, whether the rms values of the genomic bins bin_{ROI}_{ᵢ}=bin_{i,1}, . . . , bin_{i,j}, . . . , bin_{i,k}_{ᵢ}∈ROIᵢ of the Control sample significantly differ from the rms values of the according genomic bins of the Treatment sample. For this, the MEDIPS package utilises the t.test( ) and wilcox.test( ) functions of the R environment (www.R-project.org) with default parameter settings (two-sided tests in both cases). Therefore, for each tested ROIᵢ two p-values (ROI.p.value.tᵢ and ROI.p.value.wᵢ) will be calculated and serve as a further level for discriminating between local methylation profiles.

For identifying ROIᵢ's that show differential methylation between the Control and the Treatment sample and with respect to the Input sample, based on the pre-calculated parameters, a filtering procedure is performed. The following filtering procedure also discriminates between increased methylation in the Control sample compared to the Treatment sample (Control>Treatment, a) and vice versa (Treatment>Control, b):
1. ROIᵢ's where C.RMS_{ROI}_{ᵢ}=T.RMS_{ROI}_{ᵢ}=0 are neglected,
2. ROIᵢ's where ROI.p.value.tᵢ>p and ROI.p.value.wᵢ>p are neglected, where p is any targeted level of significance,
3. filtering for the ratio:
   a. ROIᵢ's where r.rms_{ROI}_{ᵢ}<h are neglected, where h is an upper ratio threshold,
   b. ROIₗ's where r.rms_{ROI}_{ᵢ}>l are neglected, where l is a lower ratio threshold,
4. filtering for global Input derived background signals:
a. ROIᵢ's where C.RPM_{ROI}_{ᵢ}<t are neglected,
b. ROIᵢ's where T.RPM_{ROI}_{ᵢ}<t are neglected,
5. filtering for local Input derived background signals:
a. ROIᵢ's where r.rpm.C_{ROI}_{ᵢ}<h are neglected,
b. ROIᵢ's where r.rpm.C_{ROI}_{ᵢ}<h are neglected.

The remaining ROIᵢ are considered as candidate genomic regions where events of differential methylation can be deduced from the data in a sophisticated statistical way.

For selecting significant regions that show de- or de-novo methylation events, we executed the MEDIPS.selectSignifieants( ) function of the MEDIPS package two times separately, and specified the following parameters: qt=0.9, up=1.333333; down=0.75, p.value=0.001. Afterwards, we ended up with highly significant candidate regions of differential methylation. Because we have executed the according MEDIPS.diffMethyl( ) function for overlapping 500 bp windows, we partly received overlapping significant frames. Therefore, we finally merged overlapping regions into one super sized region using the MEDIPS.mergeFrames( ) function of the MEDIPS package.

### REFERENCES FOR EXAMPLE 10

Boyle, A. P., Guinney, J., Crawford, G. E., and Furey, T. S. 2008. F-Seq: a feature density estimator for high-throughput sequence tags. Bioinformatics 24(21): 2537-2538.
Chavez, L., Bais, A. S., Vingron, M., Lehrach, H., Adjaye, J., and Herwig, R. 2009. In silico identification of a core regulatory network of OCT4 in human embryonic stem cells using an integrated approach. BMC Genomics 10: 314.
Down, T. A., Rakyan, V. K., Turner, D. J., Flicek, P., Li, H., Kulesha, E., Graf, S., Johnson, N., Herrero, J., Tomazou, E. M. et al. 2008. A Bayesian deconvolution strategy for immunoprecipitation-based DNA methylome analysis. Nat Biotechnol 26(7): 779-785.
Eckhardt, F., Lewin, J., Cortese, R., Rakyan, V. K., Attwood, J., Burger, M., Burton, J., Cox, T. V., Davies, R, Down, T. A. et al. 2006. DNA methylation profiling of human chromosomes 6, 20 and 22. Nat Genet 38(12): 1378-1385.
Gardiner-Garden, M. and Frommer, M. 1987. CpG islands in vertebrate genomes. J Mol Biol 196(2): 261-282.
Gentleman, RC., Carey, V. J., Bates, D. M., Bolstad, B., Dettling, M., Dudoit, S., Ellis, B., Gautier, L., Ge, Y., Gentry, J. et al. 2004. Bioconductor: open software development for computational biology and bioinformatics. Genome Biol 5(10): R80.
Ji, H., Jiang, H., Ma, W., Johnson, D. S., Myers, R M., and Wong, W. H. 2008. An integrated software system for analyzing ChIP-chip and ChIP-seq data. Nat Biotechnol 26(11): 1293-1300.
Johnson, W. E., Li, W., Meyer, C. A., Gottardo, R, Carroll, J. S., Brown, M., and Liu, X. S. 2006. Model-based analysis of tiling-arrays for ChIP-chip. Proc Natl Acad Sci USA 103(33): 12457-12462.
Kuhn, R. M., Karolchik, D., Zweig, A. S., Wang, T., Smith, K. E., Rosenbloom, K. R., Rhead, B., Raney, B. J., Pohl, A., Pheasant, M. et al. 2009. The UCSC Genome Browser Database: update 2009. Nucleic Acids Res 37(Database issue): D755-761.
Li, W., Meyer, C. A., and Liu, X. S. 2005. A hidden Markov model for analyzing ChIP-chip experiments on genome tiling arrays and its application to p53 binding sequences. Bioinformatics 21 Suppl 1: i274-282.
Lun, D. S., Sherrid, A., Weiner, B., Sherman, D. R, and Galagan, J. E. 2009. A blind deconvolution approach to high-resolution mapping of transcription factor binding sites from ChIP-seq data. Genome Biol 10(12): R142.
Pages, H. BSgenome: Infrastructure for Biostrings-based genome data packages.
Pelizzola, M., Koga, Y., Urban, A. E., Krauthammer, M., Weissman, S., Halaban, R., and Molinaro, A. M. 2008. MEDME: an experimental and analytical methodology for the estimation of DNA methylation levels based on microarray derived MeDIP-enrichment. Genome Res 18(10): 1652-1659.
Rozowsky, J., Euskirchen, G., Auerbach, R. K., Zhang, Z. D., Gibson, T., Bjornson, R., Carriero, N., Snyder, M., and Gerstein, M. B. 2009. PeakSeq enables systematic scoring of ChIP-seq experiments relative to controls. Nat Biotechnol 27(1): 66-75.
Toedling, J., Skylar, O., Krueger, T., Fischer, J. J., Sperling, S., and Huber, W. 2007. Ringo—an R/Bioconductor package for analyzing ChIP-chip readouts. BMC Bioinformatics 8: 221.
Valouev, A., Johnson, D. S., Sundquist, A., Medina, C., Anton, E., Batzoglou, S., Myers, R. M., and Sidow, A. 2008. Genome-wide analysis of transcription factor binding sites based on ChIP-Seq data. Nat Methods 5(9): 829-834.
Weber, M., Davies, J. J., Wittig, D., Oakeley, E. J., Haase, M., Lam, W. L., and Schubeler, D. 2005. Chromosome-wide and promoter-specific analyses identify sites of differential DNA methylation in normal and transformed human cells. Nat Genet 37(8): 853-862.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments encompassed by the present invention described herein. Such equivalents are intended to be encompassed by the following claims.

LENGTHY TABLES
The patent application contains a lengthy table section. A copy of the table is available in electronic form from the USPTO web site (https://seqdata.uspto.gov/?pageRequest=docDetail&DocID=US20240158864A1). An electronic copy of the table will also be available from the USPTO upon request and payment of the fee set forth in 37 CFR 1.19(b)(3).

1. A method of determining if a subject has or is at risk for developing neuroendocrine prostate cancer (NEPC), the method comprising:
detecting the presence, absence, or level of altered methylation relative to a control of one or more of the genomic loci listed in Table 5 in genomic DNA (gDNA), cell free DNA (cfDNA), and/or circulating tumor DNA (ctDNA) in a sample derived from the subject,
wherein detecting the presence, absence, or level of altered methylation comprises determining the level of methylation of the one or more genomic loci, and wherein the presence of altered methylation of the one or more of the genomic loci indicates that the subject has or is at risk for developing NEPC.

2.-3. (canceled)

4. The method of claim 1, further comprising:
(i) generating a methylation profile from the detected presence, absence, or level of methylation at the one or more genomic loci listed in Table 5; and/or
(ii) comparing the presence, absence, and/or level of methylation at the one or more of the genomic loci listed in Table 5 or the methylation profile to a control.

5. (canceled)

6. The method of claim 1, wherein the presence, absence, or level of altered methylation at the one or more genomic loci listed in Table 5 is detected by cell-free methylated DNA immunoprecipitation and high-throughput sequencing (cfMeDIP-seq) and/or is detected by whole genome bisulfite sequencing (WGBS).

7. (canceled)

8. The method of claim 1, wherein at least one of the genomic loci comprises about 50 to about 1000 nucleotides.

9.-12. (canceled)

13. The method of claim 1, wherein the genomic loci are differentially methylated regions (DMRs) relative to the same regions in a tissue control sample or a sample derived from a subject having or at risk of developing prostate adenocarcinoma (PRAD).

14. The method of claim 1, wherein the genomic loci have a predetermined area under the ROC curve (AUROC) of greater than 0.7.

15. The method of claim 1, wherein the one or more genomic loci have increased methylation relative to the same region in a tissue control sample or a sample derived from a subject having or at risk of developing PRAD, and/or wherein the one or more genomic loci have less methylation relative to the same region in a tissue control sample or a sample derived from a subject having or at risk of developing PRAD.

16.-17. (canceled)

18. The method of claim 4, further comprising determining a methylation score for the one or more genomic loci and/or the methylation profile and comparing the methylation score for the one or more genomic loci and/or the methylation profile to a predetermined threshold for each of the one or more genomic loci listed in Tables 1-8 or to a predetermined threshold for the methylation profile, wherein the predetermined threshold discriminates between NEPC and PRAD.

19. (canceled)

20. The method of claim 18, further comprising comparing the methylation score to a control, wherein a higher methylation score compared to the control indicates that the subject has or is at risk for developing NEPC, wherein the control is a reference value or a methylation score determined from a control sample.

21.-24. (canceled)

25. The method of claim 1, wherein the sample is an organ, tissue, body fluid, or cell sample.

26.-27. (canceled)

28. The method of claim 1, wherein the sample is a plasma sample, and wherein the method further comprises isolating cell-free DNA (cfDNA) or circulating tumor DNA (ctDNA) from the plasma sample.

29. The method of claim 1, further comprising administering to the subject a therapeutically effective amount of an anti-cancer therapy, wherein the anti-cancer therapy comprises one or more therapies selected from the group consisting of an epigenetic modifier, targeted therapy, chemotherapy, radiation therapy, immunotherapy, and/or hormonal therapy.

30. (canceled)

31. The method of claim 1, wherein the subject is resistant to AR-targeted therapy.

32. The method of claim 29, wherein the anti-cancer therapy comprises chemotherapy and/or immunotherapy, wherein:
(i) the chemotherapy is a platinum-based therapy;
(ii) the chemotherapy is a platinum-based therapy further comprising etoposide;
(iii) the chemotherapy is doxorubicin, etoposide, or cisplatin or combination thereof;
(iv) the immunotherapy is cell-based;
(v) the immunotherapy comprises a cancer vaccine and/or virus;
(vi) the immunotherapy comprises an immune checkpoint inhibitor that inhibits a checkpoint selected from the group consisting of CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRPalpha (CD47), CD48, 2B4 (CD244), B7.1, B7.2, ILT-2, ILT-4, TIGIT, HHLA2, butyrophilins, and A2aR; and/or
(vii) the immunotherapy comprises one or more monoclonal antibodies selected from durvalumab, atezolizumab, pembrolizumab and combinations thereof.

33.-49. (canceled)

50. A method of assessing the efficacy of an agent for treating NEPC in a subject, the method comprising:
determining in a sample derived from the subject at a first point in time the level of altered methylation relative to a control of one or more of the genomic loci listed in Table 5 in genomic DNA, cell free DNA (cfDNA), or circulating tumor DNA (ctDNA);
determining in one or more samples derived from the subject at one or more subsequent points in time the level of altered methylation relative to a control of one or more of the genomic loci listed in Tables 1-8 in genomic DNA, cell free DNA (cfDNA), or circulating tumor DNA (ctDNA);
wherein an increased aggregate level of methylation determined in the one or more subsequent samples relative to the aggregate level of methylation detected in the first sample indicates that the agent does not treat NEPC in the subject; and
wherein a decreased aggregate level of methylation determined in the one or more subsequent samples relative to the aggregate level of methylation detected in the first sample indicates that the agent treats NEPC in the subject.

51.-63. (canceled)

64. A method of treating a subject having or suspected of having NEPC, the method comprising administering to the subject a therapeutically effective amount of an agent that modulates the methylation of one or more of the genomic loci listed in Table 5, wherein the agent decreases the methylation of one or more of the genomic loci listed in Table 5 and/or increases the methylation one or more of the genomic loci listed in Table 5, thereby treating a subject afflicted with NEPC.

65.-68. (canceled)

69. The method of claim 64, further comprising administering to the subject an immunotherapy and/or cancer therapy, wherein:
(i) the cancer therapy is selected from the group consisting of radiation, a radiosensitizer, and a chemotherapy;
(ii) the cancer therapy is a platinum-based therapy
(iii) the cancer therapy is doxorubicin, etoposide, or cisplatin or combination thereof;
(iv) the immunotherapy is cell-based;
(v) the immunotherapy comprises a cancer vaccine and/or virus;
(vi) the immunotherapy comprises an immune checkpoint inhibitor that inhibits an immune checkpoint selected from the group consisting of CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRPalpha (CD47), CD48, 2B4 (CD244), B7.1, B7.2, ILT-2, ILT-4, TIGIT, HHLA2, butyrophilins, and A2aR;
(vii) the immunotherapy comprises one or more monoclonal antibodies selected from durvalumab, atezolizumab, pembrolizumab and combinations thereof.

70.-87. (canceled)

88. The method of claim 64, wherein the subject is a rodent model of NEPC or a human.

89.-93. (canceled)

94. The method of claim 64, further comprising generating an NEPC Risk Value score for the subject,
wherein an NEPC Risk Score of greater than or equal to 0.15 indicates that the subject has or is at risk for developing NEPC, wherein the NEPC Risk Value is the log₂ ratio of an NEPC Methylation Value to a PRAD Methylation Value.

95.-97. (canceled)

98. The method of claim 94, wherein the NEPC Methylation Value is calculated by summing relative methylation scores of at least seventy-six NEPC-enriched differentially methylated regions in DNA from a sample taken from the subject.

99.-110. (canceled)
